(19) United States
(12) Patent Application Publication
(10) Pub. No.: US 2026/0347781 A1
(43) Pub. Date: Nov. 5, 2026

(54) NOVEL CELL LINES AND THEIR USE IN THERAPY

(71) Applicant: The University of York, York (GB)

(72) Inventors: Paul Gilbert GENEVER, York (GB); Jennifer Ann DYSON, York (GB); Savvas IOANNOU, York (GB); Alice CARSTAIRS, York (GB)

(21) Appl. No.: 19/120,430

(22) PCT Filed: Oct. 13, 2023
(86) PCT No.: PCT/GB2023/052658

§ 371 Date: Apr. 11, 2025

(30) Foreign Application Priority Data
Oct. 13, 2022 (GB) 2215142.7

(51) Int. Cl. C12N 5/0775; A61K 35/28; A61P 19/02

(52) U.S. Cl. CPC C12N 5/0662; A61K 35/28; A61P 19/02

(57) ABSTRACT

The present invention provides novel mesenchymal stem cell (MSC)-like cells and cell populations. Associated DNA preparations, cell-free conditioned media, secretomes, extracellular vesicle populations, compositions, and uses thereof are also provided.

The present invention provides novel mesenchymal stem cell (MSC)-like cells and cell populations. Associated DNA preparations, cell-free conditioned media, secretomes, extracellular vesicle populations, compositions, and uses thereof are also provided.

### BACKGROUND

Mesenchymal stem cells (MSCs) are located in bone marrow where they give rise to skeletal tissues and interact with the immune system. The potent reparative and immunosuppressive functions of MSCs have driven their widespread clinical use as tissue regenerative and anti-inflammatory mediators. However, MSCs are almost always deployed in therapeutic settings as uncharacterised mixed stromal cell populations obtained from donor tissue (Wilson et al, 2021). This MSC heterogeneity introduces inconsistencies and clouds mechanism of action, which are essential requirements for any study drug. There are also questions over batch safety, reproducibility, scalability and transport/storage costs.

Extracellular vesicles (EVs) are nano-sized, membrane-bound carriers of proteins and nucleic acids produced and released by cells that mediate intercellular signalling. There is increasing evidence that EVs produced by MSCs can deliver many of the reparative and anti-inflammatory therapeutic benefits of the parent cells but with far fewer safety, delivery and cost limitations. EVs survive in the body for long periods without degrading or aggregating, they are acellular so present minimal risk of immunogenicity and they can be engineered to carry additional cargo, including pharmaceuticals. Consequently, there is huge interest in the development of EVs as a new therapeutic modality that combines the potency of stem cell therapies with the practicality of a biopharmaceutical. However, the key challenges to further progress are batch consistency and scalability.

Accordingly, there is a need for an improved means of delivering the benefits of MSCs in a therapeutic setting.

### BRIEF SUMMARY OF THE DISCLOSURE

To address the challenges associated with the use of MSCs in therapy, the inventors initiated a programme of immortalisation and cloning to deliver a panel of MSC lines representing different bone marrow stromal subtypes, including stem cells of varying potency. From numerous initial clones, 8 were selected based on strong in vitro growth performance for in-depth characterisation. Advantageously, the inventors identified one line in particular, termed Y201, which shows typical MSC characteristics, with potent tissue-forming and anti-inflammatory properties in vitro and in vivo. The inventors have shown that Y201 MSCs are highly reproducible and have thoroughly characterised these cells (e.g. their growth, transcriptomics, secretomics, surfaceomics, and marker expression).

The inventors have shown that Y201 MSCs produce abundant, consistent EVs for which the size, morphometric, EV marker, miRNA and proteomic data has been collected. Advantageously, the inventors have demonstrated that Y201 EVs stimulate cell growth (e.g. of donor MSCs and cartilage cells from arthritis patients), cartilage formation and suppress inflammation.

Surprisingly, further engineering of the Y201 MSCs using CRISPR/Cas9 to target a deletion of the FGFR3 gene advantageously increases proliferation, migration activity and secretory behaviour to enable growth in serum-free conditions.

Accordingly, a mesenchymal stem cell (MSC)-like cell deposited under European Collection of Authenticated Cell Cultures (“ECACC”) accession number 22072103 or a derivative thereof is provided herein.

Suitably, the derivative may have reduced FGFR3 expression and/or activity compared to a MSC-like cell deposited under ECACC accession number 22072103.

Suitably, the derivative may only differ from a MSC-like cell deposited under ECACC accession number 22072103 by having reduced FGFR3 expression and/or activity.

Suitably, the derivative may;
(a) have increased levels of at least 10 different RNA transcripts selected from Table 5 compared to the levels of the same RNA transcripts in a mesenchymal stem cell (MSC)-like cell deposited under ECACC accession number 22072103; and/or
(b) have reduced levels of at least 10 different RNA transcripts selected from Table 6 compared to the levels of the same RNA transcripts in a mesenchymal stem cell (MSC)-like cell deposited under ECACC accession number 22072103.

A mesenchymal stem cell (MSC)-like cell deposited under European Collection of Authenticated Cell Cultures (“ECACC”) accession number 22072101 or a derivative thereof is also provided.

A cell population comprising a plurality of cells provided herein is also provided.

A DNA preparation comprising the genomic DNA of a cell provided herein is also provided.

A cell-free conditioned medium that is obtainable by cell culture of a cell provided herein, or a cell population provided herein is provided.

Suitably, the cell-free conditioned medium may be depleted of extracellular vesicles (EVs).

Suitably, the cell-free conditioned medium may be substantially free of extracellular vesicles (EVs).

A secretome, or a portion thereof, that is obtainable by cell culture of a cell provided herein, or a cell population provided herein is also provided.

Suitably, the secretome, or portion thereof, may be depleted of extracellular vesicles (EVs).

Suitably, the secretome, or portion thereof, may be substantially free of extracellular vesicles (EVs).

An extracellular vesicle (EV) population that is obtainable by cell culture of a cell provided herein, or a cell population provided herein is also provided.

Suitably, the EV population may be a 100K EV fraction.

Suitably, the cell-free conditioned medium, or the secretome, or portion thereof, may comprise at least 10 different proteins selected from Table 2.

Suitably, the cell-free conditioned medium, or the secretome, or portion thereof, or the EV population, may comprise at least 10 different proteins selected from Table 3.

Suitably, the cell-free conditioned medium, or the secretome, or portion thereof, or the EV population, may comprise at least 10 different miRNA selected from Table 4.

A composition comprising at least 10 different proteins selected from Table 2 is provided.

A composition comprising at least 10 different proteins selected from Table 3 is also provided.

A composition comprising at least 10 different miRNAs selected from Table 4 is further provided.

Suitably, the composition may be a cell-free composition.

A pharmaceutical composition comprising a MSC-like cell provided herein, a cell population provided herein, a DNA preparation provided herein, a cell-free conditioned medium provided herein, a secretome, or a portion thereof, provided herein, an extracellular vesicle (EV) population provided herein, or a composition provided herein, wherein the pharmaceutical composition further comprises a pharmaceutically acceptable adjuvant, excipient, diluent and/or carrier is also provided.

A pharmaceutical composition provided herein for use as a medicament is provided.

A method of treating a disease, disorder and/or condition in a subject is also provided, the method comprising administering an effective amount of a pharmaceutical composition provided herein to a subject in need thereof.

A pharmaceutical composition provided herein for use in promoting tissue repair is also provided.

A method of promoting tissue repair in a subject is also provided, the method comprising administering an effective amount of a pharmaceutical composition provided herein to a subject in need thereof.

Suitably, the pharmaceutical composition may be for treating or preventing a disease or condition associated with cartilage damage.

Suitably, the method of promoting tissue repair in a subject may be for treating or preventing a disease or condition associated with cartilage damage.

Suitably, the disease or condition associated with cartilage damage may be arthritis, optionally the arthritis may be selected from the group consisting of: juvenile idiopathic arthritis, rheumatoid arthritis, osteoarthritis, spondyloarthritis and psoriatic arthritis.

Suitably, the pharmaceutical composition may be for use in wound healing and/or tissue regeneration.

Suitably, the method of promoting tissue repair in a subject may be for wound healing and/or tissue regeneration.

A pharmaceutical composition provided herein for use in treating or preventing inflammation is also provided.

A method of treating or preventing inflammation in a subject is also provided, the method comprising administering an effective amount of a pharmaceutical composition provided herein to a subject in need thereof.

Suitably, the pharmaceutical composition may be for use in treating or preventing an autoimmune disease or condition.

Suitably, the method of treating or preventing inflammation may be for treating or preventing an autoimmune disease or condition.

Use of an extracellular vesicle (EV) population provided herein for delivery of a cargo to a cell is also provided.

A method of screening a test compound for its ability to induce differentiation of MSC-like cells is provided herein, said method comprising: a) contacting the test compound with a MSC-like cell described herein, or a cell population described herein; and b) determining the effect of the test compound on at least one marker of differentiation.

Suitably, the at least one marker of differentiation may be selected from the group consisting of: an osteogenic differentiation marker and a chondrogenic differentiation marker, optionally wherein:
a) the osteogenic differentiation marker is selected from the group consisting of: Type I collagen, alkaline phosphatase, BMP2, osteopontin, osteonectin, osteocalcin, Bone Sialoprotein, and Runx2; and/or
b) the chondrogenic differentiation marker is selected from the group consisting of: Type II collagen, Type X collagen, aggrecan, lubricin, cartilage oligomeric matrix protein, and Sox9.

Use of a MSC-like cell described herein, or a cell population described herein, to screen test compounds for their ability to induce differentiation of MSC-like cells is also provided.

Throughout the description and claims of this specification, the words “comprise” and “contain” and variations of them mean “including but not limited to”, and they are not intended to (and do not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

Various aspects of the invention are described in further detail below.

### BRIEF DESCRIPTION OF THE FIGURES

Embodiments of the invention are further described hereinafter with reference to the accompanying drawings, in which:

FIG. 1 shows the karyotype of Y201 MSCs. Representative from 20 individual cells selected from one Y201 culture at passage 73, Independent interpretation of Y201 karyotype.

FIG. 2 shows Y201 morphology and migratory profile versus comparator MSC line MSC #2 A) Crystal violet stained MSC subtypes (scale=50 μm) B) Cell morphometrics calculated from time-lapse imaged CFU-Fs. i) Length: Width ratio ii) Cell Perimeter and iii) Cell Area. Unpaired t-test, P<0.0001, n=2418 cells C) Rose plots highlighting varied migratory profiles between Y201 and MSC #2 subsets. D) Migration metrics calculated from time-lapse images for CFU-Fs Y201 vs MSC #2 i) Distance migrated ii) Speed of cells Y201=0.413 μm/s+0.1064 MSC #2=0.2536 μm/s+0.068 E) Immunofluorescence micrograph of a representative Y201 and MSC #2 cell showing DAPI stained nuclei (Blue), Phalloidin stained actin and Vinculin to mark focal adhesions (Green) F) Mean focal adhesion area per cell of Y201 vs MSC #2 (t-test, p<0.0001, n=10-12) G) Mean focal adhesion length (t-test, p<0.0001, n=10-12) Error bars=Mean±SD*=p≤0.05, **=p<0.01, ***=p<0.001, ****=p<0.0001.

FIG. 3 shows secreted factors from Y201 MSCs alters the morphology and migration of other MSC subtypes (MSC #2). A) Representative images of gross colony morphology of MSC #2 cells cultured in either unconditioned medium (No CM), Y201-conditioned medium or MSC #2-conditioned medium. B) Mean colony area of MSC #2 colonies treated with various conditioned media (ANOVA: F=60.05, d.f=1.12, 2.26, p=0.0113) C) Length: Width ratio of cells tracked during a single repeat and the Mean Length:width ratio from multiple repeats (n=5) D) Speed of mean cell movement from a single experiment and the mean speed from multiple repeats (n=5) E) Displacement distance of cells from tracking origin for a single repeat and the mean distance from multiple repeats (n=5). F) Representative Rose plots of cell migratory pathways after exposure to Y201 or MSC #2 conditioned media. G) Phase contrast image of typical colonies at assay endpoint H) Immunofluorescent images of MSC #2 cells treated with either Y201-CM or MSC #2-CM for 24 hours. Cells were stained with DAPI for nuclear staining, Phalloidin to reveal actin and vinculin to mark focal adhesions.

FIG. 4 shows that the Y201 MSC secretome is enriched for extracellular matrix components. A) Volcano plot of proteins identified by LC-MS/MS in conditioned media from Y201 and comparator MSC line MSC #2. Proteins identified as significantly enriched by ANOVA n=3, p<0.05. B) KEGG pathway enrichment of proteins identified as significantly enriched in i) Y201 and ii) MSC #2. C) Significantly enriched proteins secreted by Y201 versus MSC #2 represented in order of overall normalized abundance from LC-MS/MS. Graphs split in for ease of interpretation while maintaining a linear scale. Means±SEM D) Significantly enriched proteins secreted by MSC #2 versus Y201 represented in order of normalized abundance from LC-MS/MS. Graphs are split for ease of interpretation while maintaining a linear scale. E) Effect of different substrates (Y201-derived ECM, MSC #2-derived ECM or plastic) on MSC #2 migratory behaviour. Means±SEM.

FIG. 5 shows the characterisation of EVs isolated from Y201 MSCs. A) Nanoparticle tracking analysis was used to determine the sizes of the 10k and 100k EV fractions from Y201 MSCs. B) Quantification of Y201 EVs versus comparator MSC sub-line MSC #2 using the LM14 Nanosight 3.4 software. C) Negatively stained Y201 EV samples imaged using TEM. D) Western blot analysis of EV markers (Flotillin-1, CD81, CD63) and negative control (BiP) Y201 and MSC #2 EVs and whole cells.

FIG. 6 shows the effect of Y201 EVs on MSC #2 proliferation A) Delivery of Y201 EVs to primary bone marrow derived MSCs. Y201 EVs were labelled with CFSE (green) and applied to MSC cultures. CFSE-labelled EV uptake was tracked by fluorescence microscopy every hour for 9 hours. Magenta=F-actin, white=nuclei. Data show CFSE fluorescence within the cells from 5 hours onwards. B)) MSC #2 cells treated with 1×, 5× and 10× concentrations of Y201 EVs and cell number determined over 72 hrs (DNA content measured as fluorescence using the CyQuant assay). Livecyte image analysis was used to determine the effect of 10× Y201 EVs on C) MSC #2 cell counts, D) MSC #2 doubling times, E) dry mass, F) dry mass doubling time, and G) MSC #2 confluence. T-test or 2-Way Anova *p<0.05, **p<0.01, ***p<0.001, ****p<0.0001.

FIG. 7 shows the effect of Y201 EVs on migration of MSC #2 cells. The scratch wound assay was used to monitor MSC #2 migration with and without exposure to Y201 EVs using Livecyte image analysis. A) Micrographs of untreated control (left) and Y201 EV-treated (right) scratch-wounds at o hours (top) and 24 hours (bottom). Effect of Y201 EVs on B) gap area, C) MSC #2 track speed, D) area T1/2, E) MSC #2 collective migration F) MSC #2 track length, G) cell velocity and H) forward migration compared to untreated controls. T-Test or 2-Way Anova *p<0.05, **p<0.01, ***p<0.001, ****p<0.0001.

FIG. 8 shows the effect of Y201 EVs on primary MSC proliferation. Primary MSCs were exposed to Y201 100k EVs at 1×, 5× and 10× concentrations and cell number measured using Alamar Blue over 3 days. Two-Way Anova followed by Tukey multiple comparison **p<0.01, ***p<0.001, ****p<0.0001

FIG. 9 shows the effect of Y201 EVs on proliferation of primary articular chondrocytes. Primary chondrocytes were exposed to Y201 100k EVs at 10× and 20× concentrations and cell number measured by Livecyte image analysis over 72 hours showing A) confluence, B) Total dry mass, C) cell counts and D) doubling times. E) Western blot of MFG-E8 in Y201 EVs (100k fraction), MSC #2 EVs, Y201 cells and MSC #2 cells. L=molecular weight ladder. F) Effect of Y201 EVs on proliferation of primary osteoarthritic articular chondrocytes, with and without RGD blocking peptides (GRGDSP-SEQ ID NO. 16) or RAD control peptides (GRADSP-SEQ ID NO. 17). Two-Way-Anova followed by Tukey multiple comparison *p<0.05, **p<0.01, ***p<0.001 ****p<0.0001

FIG. 10 shows the effect of Y201 EVs on chondrogenesis. Primary donor MSCs as micromass pellets in basal or chondrogenic differentiation conditions for 7 days with or without exposure to Y201 EVs. Micromasses were fixed, sectioned and stained with Safranin O. Red staining indicates chondrogenic differentiation. A) Day 0 control, B) Day 7, no EV control, C) Day 7 plus Y201 EVs, D) Day 7, no EV control, basal non-differentiating conditions, E) Day 7 plus Y201 EVs, basal non-differentiating conditions. Data show increased micromass size and red staining/chondrogenesis following treatment with Y201 EVs.

FIG. 11 shows the immunomodulatory capacity of EVs isolated from Y201 MSCs. A) In vitro treatment of activated CD4+ T cells with Y201 cells and Y201-EVs showing effects on the proportion of proliferating T cells and number of cell cycles achieved (n=2, mean events >18,000 counted). B) Representative images of proliferative generations in activated T cells alone and treated with Y201 cells or their EVs. C and D) Separate plots of proliferative cycles and proliferative index respectively. E) Polarisation of activated T cells in the absence and presence of Y201-EVs (n=2, mean events >32,000 counted).

FIG. 12 relates to A, B) Peritoneal exudate cell counts following stimulation with zymosan (A) or schistosome eggs (B) in the absence and presence of Y201-EVs). C, D) Examination of TCR+CD4+, naïve and central memory T cells in zymosan or schistosome egg induced peritoneal inflammation with and without Y201-EVs (n=3). 1-way ANOVA with Bonferroni post hoc testing, *p<0.05, **p<0.01, ***p<0.001

FIG. 13 shows a comparison of the colony-forming capacity of WT and FGFR3-KO MSCs. CFU efficiency (A), average colony surface area (B) and total colony surface area (C) were not significantly different between the lines. Bars show mean±SEM, ns=p>0.05, as determined by t-tests (with Welch's correction for B), n=3 biological replicates.

FIG. 14 shows crystal violet staining of WT and FGFR3-KO MSC colonies. Well-view of WT (A) and FGFR3-KO (B) CFU-F plates. Individual colony view of WT (C) and FGFR3-KO (D) cells.

FIG. 15 shows a comparison of WT and FGFR3-KO MSC proliferation rates. Alamar blue cell viability assay fluorescence readings at 600 nm, relative to fluorescence of WT cells at day 0 (A). Cell counts of WT and FGFR3-KO MSCs (B). Bars show mean±SEM, n=6 (A) and n=3 (B). ****=p<0.0001, as determined by Sidak's multiple comparisons test.

FIG. 16 shows WT and FGFR3-KO MSC proliferation during continuous culture. Cumulative cell count (A) and population doublings (B) of WT and FGFR3-KO MSCs over time. Mean±SEM plotted, n=3 biological replicates.

FIG. 17 shows a comparison of WT and FGFR3-KO MSC morphologies. Crystal violet stained brightfield images of WT (A) and FGFR3-KO (B) MSCs. Area (C), showing median, inter-quartile, and min-max values. Length to width ratio (D), and roundness index (E), showing mean±SEM. ****=p<0.0001, as determined by Mann-Whitney test, n=1032 for WT, and n=345 for FGFR3-KO MSCs.

FIG. 18 shows the top ten significantly enriched KEGG pathways for genes downregulated (A) and upregulated (B) in FGFR3-KO MSCs versus WT. All pathways have p values of <0.05, q value of 0.05 is indicated by red lines.

FIG. 19 shows the top ten significantly enriched Cellular Component Gene Ontology (GO) terms for genes downregulated (A) and upregulated (B) in FGFR3-KO MSCs versus WT. All GO terms have Q values of <0.05.

FIG. 20 shows the top ten significantly enriched Biological Processes Gene Ontology (GO) terms for genes downregulated (A) and upregulated (B) in FGFR3-KO MSCs versus WT. All GO terms have Q values of <0.05.

FIG. 21 shows the top ten significantly enriched Molecular Functions Gene Ontology (GO) terms for genes downregulated (A) and upregulated (B) in FGFR3-KO MSCs versus WT.

FIG. 22 shows the visualisation of the WT and FGFR3-KO MSC actin cytoskeleton. Representative confocal images of WT (A, B) and FGFR3-KO (C, D) MSCs, stained with Alexa Fluor 594-conjugated Phalloidin, marking actin (red), and DAPI, marking nuclei (blue). Scale bars equivalent to 20 μm.

FIG. 23 shows a comparison of WT and FGFR3-KO MSC actin cytoskeleton shape. Roundness (A) and length to width ratio (B) of WT and FGFR3-KO MSCs. ****=p<0.0001; Mann Whitney test.

FIG. 24 shows the visualisation of Arp3 protein in WT and FGFR3-KO MSCs. Immunofluorescence of WT (A, C, E) and FGFR3-KO (B, D, F) MSCs, with staining for Arp3 (green) actin (red) and nuclei (blue). Scale bar=20 μm

FIG. 25 shows the scratch closure of WT and FGFR3-KO MSCs after 24 hours of wound healing. Bars show mean+SEM, *=p<0.05, t test, n=5.

FIG. 26 shows the migratory characteristics of WT and FGFR3-KO MSCs. Gap closure of WT and FGFR3-KO MSCs after 24 hours of migration (A). Total track length (B) and instantaneous velocity (C) of individual cells throughout migration. Number of cell divisions (D) within each field of view during migration. Area (E), sphericity (F) and directness (G) of individual cells throughout migration. Still images of WT (H) and FGFR3-KO (I) MSCs at the end of the 24 hour migration, with lines denoting the initial gap space. Boxplots show minimum, maximum, and quartile values. Bars show mean±SEM. ****=p<0.0001; **=p<0.01; *=p<0.05, as determined by Mann Whitney tests for A-C, E, F, and by t tests for D, G.

FIG. 27 shows nanoparticle tracking analysis (NTA) of extracellular vesicles (EVs) from WT and FGFR3-KO MSCs. Histogram overlays of EV size distribution for 2k (A), 10k (B), and 100k (C) fractions. Lines show mean±SEM of 5 technical replicates. Lower line=WT, upper line=FGFR3-KO.

FIG. 28 shows representative transmission electron micrographs of 100k extracellular vesicles from WT and FGFR3-KO MSCs. EVs isolated from WT (A, B) and FGFR3-KO (C-F) MSCs. Scale bars as indicated on individual panels.

FIG. 29 shows cell migration metrics following scratch wounding of WT MSCs treated with components of the FGFR3-KO MSC secretome. Percentage scratch closure after 24 hours (A), total track length (B), directness index (C), and mean cell thickness (D). Instantaneous velocity throughout the timecourse (E). Bars show mean±SEM. Lines show means. ****=p<0.0001; **=p<0.01; *=p<0.05, Tukey's multiple comparisons test (A), Dunn's multiple comparisons test (B-D).

FIG. 30 shows the effect of WT and FGFR3-KO conditioned media (CM) on WT MSC wound healing, compared to FGFR3-KO MSC healing. ****=p<0.0001; **=p<0.01; ns=p>0.05; Dunn's multiple comparisons test. Bars show mean±SEM.

FIG. 31 shows the effect of conditioned media (CM) on MSC morphology and proliferation. WT or FGFR3-KO (KO) MSCs were treated with CM derived from WT (+WT CM) or FGFR3-KO (+KO CM) MSCs, and alamar blue cell viability assay fluorescence readings taken at 600 nm, shown relative to fluorescence of WT cells at day 0, key shows order of bars on the graph (A). On day 2 of treatments, cell area (B), length:width ratio (C) and roundness (D) were calculated. Bars show mean±SEM, boxplots show 1-99 percentiles, and each quartile. No significant differences in A according to Tukey's multiple comparisons test. Significant differences relative to each untreated control shown for B-D, according to Dunn's multiple comparisons test: ****=p<0.0001; *=p<0.05.

FIG. 32 shows the effect of FGFR3-KO extracellular vesicles (EVs) on WT MSC proliferation. Alamar blue cell viability assay fluorescence readings 600 nm, shown relative to fluorescence of WT cells at day 0. Key shows order of lines on the graph, top to bottom. Mean+SEM of 3 biological replicates shown for B, no significant differences according to Sidak's multiple comparisons test.

FIG. 33 shows proliferation and morphology of WT and FGFR3-KO MSCs on serum free (0% FBS) medium. Alamar blue cell viability assay (A) of WT and FGFR3-KO (KO) MSCs cultured in control or serum free (SF) medium. Fluorescence relative to WT MSCs at day 0. Cumulative cell count (B) of WT and FGFR3-KO MSCs, arrows indicating expansion into additional culture flasks. Points show means±SEM. Key shows order of lines on the graph, top to bottom. Brightfield images of crystal violet stained WT MSCs, cultured in complete (C) or SF (D) medium, and FGFR3-KO MSCs cultured in complete (E) or SF (F) medium. Scale bars=200 μm.

FIG. 34 shows the functional enrichment analysis of the Y201 EV proteome (100k fraction). A) Enrichment analysis against Biological Process, B) Molecular Function and C) Cellular Component Gene Ontology (GO) terms for the Y201 proteome. Only the most significant terms are shown, as determined by −log 10 adjusted p-value (FDR) and number of proteins associated with each term. Protein count is as indicated. (D) BINGO network of significant GO Biological Process terms for the Y201 proteome, annotated to cluster common terms by similar processes. Darker shades correspond to higher significance (p-value threshold <0.05).

FIG. 35 shows A) STRING PPI network detailing relative abundance and strength of interactions between proteins in the Y201 core proteome at the EV surface (100k fraction). Node size was mapped to the relative abundance of proteins; Edge width corresponds to combined score of the interaction. Network visualisation created using BioRender.com. B) Western blot analysis of Fibronectin-1 (FN1) and MFG-E8 in Y201 (100k fraction) and MSC #2 EVs and cell lysates.

FIG. 36. shows the uptake of Y201 EVs (100k fraction) by MSC #2 cells. A) MSC #2 cells were treated with 10×CFSE-Y201 EVs, and CFSE signal in the cells was monitored for 10 hrs. Histograms (counts vs CFSE) signals were normalised to peak values, dashed lines represent the median values used to quantify CFSE signal levels relative to control (cells). B) Quantification of the median CFSE intensity relative to control were plotted as bar graphs. n=3, error bars=SEM, Kruskal-Wallis test, *p<0.05, **p<0.01, ***p<0.001.

FIG. 37 shows the uptake of CFSE-labelled Y201 EVs (100k fraction) by MSC #2 cells with and without RGD blocking peptides (GRGDSP—SEQ ID NO. 16) or RAD control peptides (GRADSP—SEQ ID NO. 17). A) Histograms against CFSE signal demonstrating the population shift after cells were exposed with Y201 labelled EVs for 4 hr. B) Fold change quantification of the CFSE median fluorescence of the MSC #2 cells. n=6, error bars=SEM, 1-Way ANOVA with Bonferroni corrections.

FIG. 38 shows the effect of Y201 EVs (100k fraction) on disease activity in an in vivo adjuvant-induced arthritis model. A) Joint size reduction (mm), B) synovial infiltrate score, C) joint exudate score, D) synovial hyperplasia score, E) arthritis index. Mice were terminated 3 days post-treatment and sections were scored by two independent scorers. Haematoxylin and eosin-stained sections of F and I) PBS control, G and J) Y201 EV-treated mouse knee joints. Boxed areas in F and G are enlarged in I and J respectively showing examples of synovial infiltrate (cellular infiltration into synovium; arrows) and synovial hyperplasia (thickened synovial lining; arrowheads). Scale bars=500 mm. Mean values shown±SD (n=4) Unpaired t-test with Welch's correction, *p<0.05, **p<0.01, ***p<0.001.

The patent, scientific and technical literature referred to herein establish knowledge that was available to those skilled in the art at the time of filing. The entire disclosures of the issued patents, published and pending patent applications, and other publications that are cited herein are hereby incorporated by reference to the same extent as if each was specifically and individually indicated to be incorporated by reference. In the case of any inconsistencies, the present disclosure will prevail.

Various aspects of the invention are described in further detail below.

BUDAPEST TREATY ON THE INTERNATIONAL RECOGNITION OF THE DEPOSIT OF MICROORGANISMS FOR THE PURPOSE OF PATENT PROCEDURES The cell lines described in this application were deposited with the United Kingdom Health Security Agency Porton Down and European Collection of Authenticated Cell Cultures (ECACC), which is an International Depositary Authority, located at UK Heath Security Agency, Culture Collections, Porton Down, Salisbury, SP4 0JG, UK.

The deposits were made under the terms of the Budapest Treaty on the International Recognition of the Deposit of Microorganisms for the Purposes of Patent Procedure.

### DETAILED DESCRIPTION

As discussed above, the inventors have identified a novel MSC-like cell line, termed Y201, which shows typical MSC characteristics, advantageously, with potent tissue-forming and anti-inflammatory properties in vitro and in vivo. The inventors have shown that Y201 MSCs are highly reproducible and have thoroughly characterised these cells (e.g. their growth, transcriptomics, secretomics, surfaceomics, and marker expression).

The inventors have shown that Y201 MSCs produce abundant, consistent EVs for which the size, morphometric, EV marker, miRNA and proteomic data has been collected. Advantageously, the inventors have demonstrated that Y201 EVs stimulate cell growth (e.g. of donor MSCs and cartilage cells from arthritis patients), cartilage formation and suppress inflammation.

Surprisingly, the inventors have demonstrated that further engineering of the Y201 MSCs using CRISPR/Cas9 to target a deletion of the FGFR3 gene advantageously increases proliferation, migration activity and secretory behaviour to enable growth in serum-free conditions.

Accordingly, a mesenchymal stem cell (MSC)-like cell deposited under ECACC accession number 22072103 or a derivative thereof is provided herein.

A mesenchymal stem cell (MSC)-like cell deposited under ECACC accession number 22072103 may be referred to herein as a “Y201 cell”, “(Y201) WT MSC”, and/or a “Y201 MSC”. A Y201 cell (deposited under ECACC accession number 22072103) was deposited at the UK Health Security Agency Porton Down and European Collection of Authenticated Cell Cultures, Porton Down, Salisbury, SP4 OJG, UK under The Budapest Treaty of 1977 on 21 Jul. 2022.

Mesenchymal stem cells (MSCs), are multipotent stromal cells which have the potential to differentiate into a variety of mesenchymal cell types of the adipocytic, chondrocytic and osteocytic lineages, including: osteoblasts, chondrocytes, neurons, muscle cells and adipocytes. This potential has been documented in specific cells and tissues in vivo and in vitro. Mesenchymal stem cells (MSCs) are located in bone marrow where they give rise to skeletal tissues and interact with the immune system. MSCs have potent reparative and immunosuppressive functions which has driven their widespread clinical use as tissue regenerative and anti-inflammatory mediators. MSCs are plastic-adherent, migratory and form colonies (CFU-Fs) when plated at low seeding densities. MSCs also show strong growth and stable phenotype post-immortalisation. MSCs cells may be defined phenotypically by gene or protein expression. MSCs have been characterized to express (and thus be positive for) one or more of CD13, CD29, CD44, CD49a, b, c, e, f, CD51, CD54, CD58, CD71, CD73, CD90, CD102, CD105, CD106, CDw119, CD120a, CD120b, CD123, CD124, CD126, CD127, CD140a, CD166, P75, TGF-bIR, TGF-blIR, HLA-A, B, C, SSEA-3, SSEA-4, D7 and PD-L1. These cells have also been characterized as not expressing (and thus being negative for) one or more of CD3, CD5, CD6, CD9, CD10, CD11a CD14, CD15, CD18, CD21, CD25, CD31, CD34, CD36, CD38, CD45, CD49d, CD50, CD62E, L, S, CD80, CD86, CD95, CD117, CD133, SSEA-1, and ABO. Thus, mesenchymal stem cells may be characterized phenotypically and/or functionally (for example, according to their differentiative potential and/or their functionality (e.g. their reparative or immunosuppressive functions)).

As used herein, a “mesenchymal stem cell (MSC)-like cell” refers to a cell that is a clonally selected, immortalised cell, sharing one or more markers and/or behavioural characteristics associated with typical MSCs. For example, an MSC-like cell may exhibit a stem-cell like phenotype, e.g. differentiation potential. As described elsewhere herein, the inventors have identified that cells deposited under ECACC accession number 22072103 (i.e. Y201 cells) show typical MSC characteristics in vitro and in vivo, in particular, potent tissue-forming and anti-inflammatory properties as demonstrated by in vitro tri-lineage differentiation, proliferation and T cell activation assays, in vivo subcutaneous implant assays and peritonitis models (see also examples section below). Accordingly, Y201 cells as described herein are an example of a MSC-like cell. In another example, as described in more detail elsewhere herein, the inventors have identified that cells deposited under ECACC accession number 22072101 (i.e. Y201 FGFR3 KO cells) also show some typical MSC characteristics in vitro, in particular, their marker expression, plastic-adherence, and colony-forming potential as demonstrated using flow cytometry and image analysis (see also examples section below). Accordingly, Y201 FGFR3 KO cells as described in more detail below are also an example of a MSC-like cell. A person of skill in the art would readily be able to identify a mesenchymal stem cell (MSC)-like cell using routine methods known in the art, for example, using the methods described above, as well as those described in the examples section below. A further example of a MSC-like cell is the MSC #2 cell line that is used as a comparator cell line in the examples section below.

The term “derivative” as used herein in the context of a cell refers to a progeny cell that has been derived from a “parent” cell, where the progeny cell is genetically distinct from the parent cell. In other words, a derivative is a modified version of the original (parent) cell from which the derivative is derived. The derivative may be modified by any known means in the art, by natural or non-natural means (including genetic engineering). A derivative cell as described herein may be derived from (e.g. generated from) a mesenchymal stem cell (MSC)-like cell deposited under ECACC accession number 22072103. An example of a derivative that has been derived from a mesenchymal stem cell (MSC)-like cell deposited under ECACC accession number 22072103 is a mesenchymal stem cell (MSC)-like cell deposited under ECACC accession number 22072101. In this context, the mesenchymal stem cell (MSC)-like cell deposited under ECACC accession number 22072103 is the “parent” cell, and the mesenchymal stem cell (MSC)-like cell deposited under ECACC accession number 22072101 is the modified progeny cell.

A derivative cell as described herein may be derived from (e.g. generated from) a mesenchymal stem cell (MSC)-like cell deposited under ECACC accession number 22072101. In this context, the derivative cell would be a modified version (modified progeny cell) of the mesenchymal stem cell (MSC)-like cell deposited under ECACC accession number 22072101 (parent cell).

In one example, a derivative (e.g. a derivative of a mesenchymal stem cell (MSC)-like cell deposited under ECACC accession number 22072103, or a derivative of a mesenchymal stem cell (MSC)-like cell deposited under ECACC accession number 22072101) may have reduced expression and/or activity of a particular protein compared to the cell from which the derivative was derived (e.g. the MSC-like cell deposited under ECACC accession number 22072103, or a derivative of a mesenchymal stem cell (MSC)-like cell deposited under ECACC accession number 22072101).

As discussed elsewhere herein, the inventors modified Y201 cells using CRISPR/Cas9 to target a deletion of the FGFR3 gene and determine effects on cell behaviour. The inventors surprisingly found that such further engineering of Y201 MSCs advantageously increases proliferation, migration activity and secretory behaviour of the engineered cells and further enables growth in serum-free conditions.

The inventors have therefore identified that it is advantageous to modify Y201 cells to generate a knockout with FGFR3 as a target. Accordingly, in some examples, the derivative (i.e. a derivative of a mesenchymal stem cell (MSC)-like cell deposited under ECACC accession number 22072103) has reduced FGFR3 expression and/or activity compared to a MSC-like cell deposited under ECACC accession number 22072103.

In a further example, the derivative (i.e. a derivative of a mesenchymal stem cell (MSC)-like cell deposited under ECACC accession number 22072103) only differs from a MSC-like cell deposited under ECACC accession number 22072103 by having reduced FGFR3 expression and/or activity.

Fibroblast Growth Factor Receptor 3 (FGFR3; gene ID 2261) is an example of a fibroblast growth factor receptor (FGFR) which are high-affinity receptors for the fibroblast growth factors. These factors have a diverse role in cell growth, differentiation and other biological processes, their precise function being dependent on the target cell and development stage. The FGFR3 gene is located on the short arm of chromosome 4 and encodes a receptor tyrosine kinase. The FGFR3 protein has a transmembrane domain in the central portion, a tyrosine kinase domain on the carboxyl-terminal, and an extracellular domain on the amino-terminal. FGFR3 is known to have isoforms including FGFR-IIIb and FGFR-IIIc resulting from alternative splicing conducted on the amino-terminal. FGF-1 and FGF-9 are ligands of FGFR3b (FGFR-IIIb), and FGF-1, FGF-2, FGF-4, FGF-8, FGF-9, FGF-17, FGF-18, and FGF-23 are ligands of FGFR3c (FGFR-IIIc).

As used herein, the term “FGFR3” can include both mutated and wild-type forms, isoforms and variants thereof (e.g. human FGFR3 polypeptides (e.g., human FGFR3-IIIb isoform or the human FGFR3-IIIc isoform). The italicized term, “FGFR3” as used herein typically refers to the FGFR3 gene.

The term “wild-type sequence” specifically encompasses naturally occurring truncated forms (e.g., an extracellular domain sequence or a transmembrane subunit sequence), naturally occurring variant forms (e.g., alternatively spliced forms) and naturally-occurring allelic variants. The term “wild-type FGFR3” generally refers to a gene or polypeptide comprising an amino acid sequence of a naturally occurring FGFR3 gene or protein. For example, in the context of the invention, gene ID 2261, which is present in Y201 cells, may be considered a wild-type FGFR3. The term “mutation”, as used herein, means a difference in the amino acid or nucleic acid sequence of a particular protein or nucleic acid (e.g. a gene, or an RNA) relative to the wild-type protein or nucleic acid, respectively. A mutated protein or nucleic acid can be expressed from or found on one allele (heterozygous) or both alleles (homozygous) of a gene. Mutations may include sequence rearrangements such as insertions, deletions, and point mutations (including single nucleotide/amino acid polymorphisms).

In some examples, FGFR3 expression and/or activity is reduced by at least 20%, at least 30%, at least 40%, at least 50%, at least 60%, at least 70%, at least 80%, at least 90%, at least 95%, at least 96%, at least 97%, at least 98%, at least 99% or 100% as compared to a MSC-like cell deposited under ECACC accession number 22072103. In some examples, FGFR3 expression and/or activity is reduced by 20%, 30%, 40%, 50%, 60%, 70%, 80%, 90%, 95%, 96%, 97%, 98%, 99% or 100% as compared to a MSC-like cell deposited under ECACC accession number 22072103. In another example, FGFR3 expression and/or activity is reduced by at least 50% as compared to a MSC-like cell deposited under ECACC accession number 22072103. In a further example, FGFR3 expression and/or activity is reduced by at least 70% as compared to a MSC-like cell deposited under ECACC accession number 22072103.

A reduction in FGFR3 expression in a cell may occur at a nucleic acid or protein level (e.g. at the level of the gene, transcript (e.g. pre-mRNA transcript, mature mRNA transcript, or cDNA transcript) and/or protein). Methods for determining FGFR3 expression are well known in the art, and include, for example western blotting. Methods for reducing FGFR3 expression in a cell are well known in the art, and include using CRISPR/Cas9, gene mutation, or RNAi. In some examples, the amount of FGFR3 expression is reduced by at least 20%, at least 30%, at least 40%, at least 50%, at least 60%, at least 70%, at least 80%, at least 90%, at least 95%, at least 96%, at least 97%, at least 98%, at least 99% or 100% as compared to a MSC-like cell deposited under ECACC accession number 22072103. In some examples, the amount of FGFR3 expression is reduced by 20%, 30%, 40%, 50%, 60%, 70%, 80%, 90%, 95%, 96%, 97%, 98%, 99% or 100% as compared to a MSC-like cell deposited under ECACC accession number 22072103. In another example, FGFR3 expression is reduced by at least 50% as compared to a MSC-like cell deposited under ECACC accession number 22072103. In a further example, FGFR3 expression is reduced by at least 70% as compared to a MSC-like cell deposited under ECACC accession number 22072103.

A reduction in FGFR3 activity in a cell may occur due to e.g. a reduction in the amount of FGFR3 protein in the cell (e.g. due to a reduction in FGFR3 expression, an increase in FGFR3 degradation, and/or a FGFR3 mutation resulting in a reduction in FGFR3 protein). A reduction in FGFR3 activity in a cell may also (or alternatively) occur due to e.g. a reduction in the amount of functional FGFR3 protein in the cell (e.g. due to a reduction in the ability of FGFR3 to interact or bind to its ligand, and/or activate FGFR3 signaling). Methods for determining FGFR3 activity are well known in the art, and include, for example, measuring phosphorylation of ERK1/2 by western blotting. Methods for reducing FGFR3 activity in a cell are well known in the art, and include using inhibitors that directly target FGFR3 (e.g. by blocking its interaction with its ligand). In some examples, the amount of FGFR3 activity is reduced by at least 20%, at least 30%, at least 40%, at least 50%, at least 60%, at least 70%, at least 80%, at least 90%, at least 95%, at least 96%, at least 97%, at least 98%, at least 99% or 100% as compared to a MSC-like cell deposited under ECACC accession number 22072103. In some examples, the amount of FGFR3 activity is reduced by 20%, 30%, 40%, 50%, 60%, 70%, 80%, 90%, 95%, 96%, 97%, 98%, 99% or 100% as compared to a MSC-like cell deposited under ECACC accession number 22072103. In another example, FGFR3 activity is reduced by at least 50% as compared to a MSC-like cell deposited under ECACC accession number 22072103. In a further example, FGFR3 activity is reduced by at least 70% as compared to a MSC-like cell deposited under ECACC accession number 22072103.

In some examples, (e.g. where the derivative has reduced FGFR3 expression and/or activity compared to a MSC-like cell deposited under ECACC accession number 22072103 or, the derivative only differs from a MSC-like cell deposited under ECACC accession number 22072103 by having reduced FGFR3 expression and/or activity) the derivative may have increased levels of at least 10 different RNA transcripts selected from Table 5 compared to the levels of the same RNA transcripts in a mesenchymal stem cell (MSC)-like cell deposited under ECACC accession number 22072103. In other words, at least 10 different RNA transcripts selected from Table 5 may be upregulated in the derivative (e.g, wherein the derivative has reduced FGFR3 expression and/or activity compared to a MSC-like cell deposited under ECACC accession number 22072103 or, wherein the derivative only differs from a MSC-like cell deposited under ECACC accession number 22072103 by having reduced FGFR3 expression and/or activity) compared to the levels of the same RNA transcripts in a mesenchymal stem cell (MSC)-like cell deposited under ECACC accession number 22072103.

In some examples, (e.g. where the derivative has reduced FGFR3 expression and/or activity compared to a MSC-like cell deposited under ECACC accession number 22072103 or, where the derivative only differs from a MSC-like cell deposited under ECACC accession number 22072103 by having reduced FGFR3 expression and/or activity) the derivative may have increased levels of at least 10, at least 20, at least 30, at least 40, at least 50, at least 60, at least 70, at least 80, at least 90, at least 100, at least 110, at least 120, at least 130, at least 140, at least 150, at least 160, at least 170, at least 180, at least 190 or 200 different RNA transcripts selected from Table 5 compared to the levels of the same RNA transcripts in a mesenchymal stem cell (MSC)-like cell deposited under ECACC accession number 22072103. For example, the derivative may have increased levels of at least 10, at least 20, at least 30, at least 40 or at least 50, different RNA transcripts selected from Table 5 compared to the levels of the same RNA transcripts in a mesenchymal stem cell (MSC)-like cell deposited under ECACC accession number 22072103.

In some examples, (e.g. where the derivative has reduced FGFR3 expression and/or activity compared to a MSC-like cell deposited under ECACC accession number 22072103 or, where the derivative only differs from a MSC-like cell deposited under ECACC accession number 22072103 by having reduced FGFR3 expression and/or activity) the derivative may have increased levels of 10, 20, 30, 40, 50, 60, 70, 80, 90, 100, 110, 120, 130, 140, 150, 160, 170, 180, 190 or 200 different RNA transcripts selected from Table 5 compared to the levels of the same RNA transcripts in a mesenchymal stem cell (MSC)-like cell deposited under ECACC accession number 22072103. For example, the derivative may have increased levels of 10, 20, 30, 40 or 50 different RNA transcripts selected from Table 5 compared to the levels of the same RNA transcripts in a mesenchymal stem cell (MSC)-like cell deposited under ECACC accession number 22072103.

In some examples, (e.g. where the derivative has reduced FGFR3 expression and/or activity compared to a MSC-like cell deposited under ECACC accession number 22072103 or, the derivative only differs from a MSC-like cell deposited under ECACC accession number 22072103 by having reduced FGFR3 expression and/or activity) the derivative may have reduced levels of at least 10 different RNA transcripts selected from Table 6 compared to the levels of the same RNA transcripts in a mesenchymal stem cell (MSC)-like cell deposited under ECACC accession number 22072103. In other words, at least 10 different RNA transcripts selected from Table 6 may be downregulated in the derivative (e.g, wherein the derivative has reduced FGFR3 expression and/or activity compared to a MSC-like cell deposited under ECACC accession number 22072103 or, wherein the derivative only differs from a MSC-like cell deposited under ECACC accession number 22072103 by having reduced FGFR3 expression and/or activity) compared to the levels of the same RNA transcripts in a mesenchymal stem cell (MSC)-like cell deposited under ECACC accession number 22072103.

In some examples, (e.g. where the derivative has reduced FGFR3 expression and/or activity compared to a MSC-like cell deposited under ECACC accession number 22072103 or, where the derivative only differs from a MSC-like cell deposited under ECACC accession number 22072103 by having reduced FGFR3 expression and/or activity) the derivative may have reduced levels of at least 10, at least 20, at least 30, at least 40, at least 50, at least 60, at least 70, at least 80, at least 90, at least 100, at least 110, at least 120, at least 130, at least 140, at least 150, at least 160, at least 170, at least 180, at least 190 or 200 different RNA transcripts selected from Table 6 compared to the levels of the same RNA transcripts in a mesenchymal stem cell (MSC)-like cell deposited under ECACC accession number 22072103. For example, the derivative may have reduced levels of at least 10, at least 20, at least 30, at least 40 or at least 50, different RNA transcripts selected from Table 6 compared to the levels of the same RNA transcripts in a mesenchymal stem cell (MSC)-like cell deposited under ECACC accession number 22072103.

In some examples, (e.g. where the derivative has reduced FGFR3 expression and/or activity compared to a MSC-like cell deposited under ECACC accession number 22072103 or, where the derivative only differs from a MSC-like cell deposited under ECACC accession number 22072103 by having reduced FGFR3 expression and/or activity) the derivative may have reduced levels of 10, 20, 30, 40, 50, 60, 70, 80, 90, 100, 110, 120, 130, 140, 150, 160, 170, 180, 190 or 200 different RNA transcripts selected from Table 6 compared to the levels of the same RNA transcripts in a mesenchymal stem cell (MSC)-like cell deposited under ECACC accession number 22072103. For example, the derivative may have reduced levels of 10, 20, 30, 40 or 50 different RNA transcripts selected from Table 6 compared to the levels of the same RNA transcripts in a mesenchymal stem cell (MSC)-like cell deposited under ECACC accession number 22072103.

As would be clear to the skilled person, in some examples (e.g, wherein the derivative has reduced FGFR3 expression and/or activity compared to a MSC-like cell deposited under ECACC accession number 22072103 or, wherein the derivative only differs from a MSC-like cell deposited under ECACC accession number 22072103 by having reduced FGFR3 expression and/or activity) the derivative may have increased levels of a plurality of RNA transcripts (e.g. RNA transcripts selected from Table 5) compared to the levels of the same RNA transcripts in a mesenchymal stem cell (MSC)-like cell deposited under ECACC accession number 22072103 and also reduced levels of a plurality of RNA transcripts (e.g. RNA transcripts selected from Table 6) compared to the levels of the same RNA transcripts in a mesenchymal stem cell (MSC)-like cell deposited under ECACC accession number 22072103. In this context, it would be clear to a person of skill in the art, that the derivative may have any combination of these features (e.g. any combination of (i) increased levels of a plurality of RNA transcripts selected from Table 5 and (ii) reduced levels of a plurality of RNA transcripts selected from Table 6 compared to the levels of the same RNA transcripts in a mesenchymal stem cell (MSC)-like cell deposited under ECACC accession number 22072103).

Accordingly, in some examples, (e.g. where the derivative has reduced FGFR3 expression and/or activity compared to a MSC-like cell deposited under ECACC accession number 22072103 or, where the derivative only differs from a MSC-like cell deposited under ECACC accession number 22072103 by having reduced FGFR3 expression and/or activity) the derivative may:
(a) have increased levels of at least 10 different RNA transcripts (e.g. 10 different RNA transcripts) selected from Table 5 compared to the levels of the same RNA transcripts in a mesenchymal stem cell (MSC)-like cell deposited under ECACC accession number 22072103; and/or
(b) have reduced levels of at least 10 different RNA transcripts (e.g. 10 different RNA transcripts) selected from Table 6 compared to the levels of the same RNA transcripts in a mesenchymal stem cell (MSC)-like cell deposited under ECACC accession number 22072103.

For example, the derivative may:
(a) have increased levels of at least 20 different RNA transcripts (e.g. 20 different RNA transcripts) selected from Table 5 compared to the levels of the same RNA transcripts in a mesenchymal stem cell (MSC)-like cell deposited under ECACC accession number 22072103; and/or
(b) have reduced levels of at least 20 different RNA transcripts (e.g. 20 different RNA transcripts) selected from Table 6 compared to the levels of the same RNA transcripts in a mesenchymal stem cell (MSC)-like cell deposited under ECACC accession number 22072103.

In another example, the derivative may:
(a) have increased levels of at least 30 different RNA transcripts (e.g. 30 different RNA transcripts) selected from Table 5 compared to the levels of the same RNA transcripts in a mesenchymal stem cell (MSC)-like cell deposited under ECACC accession number 22072103; and/or
(b) have reduced levels of at least 30 different RNA transcripts (e.g. 30 different RNA transcripts) selected from Table 6 compared to the levels of the same RNA transcripts in a mesenchymal stem cell (MSC)-like cell deposited under ECACC accession number 22072103.

In another example, the derivative may:
(a) have increased levels of at least 40 different RNA transcripts (e.g. 40 different RNA transcripts) selected from Table 5 compared to the levels of the same RNA transcripts in a mesenchymal stem cell (MSC)-like cell deposited under ECACC accession number 22072103; and/or
(b) have reduced levels of at least 40 different RNA transcripts (e.g. 40 different RNA transcripts) selected from Table 6 compared to the levels of the same RNA transcripts in a mesenchymal stem cell (MSC)-like cell deposited under ECACC accession number 22072103.

In another example, the derivative may:
(a) have increased levels of at least 50 different RNA transcripts (e.g. 50 different RNA transcripts) selected from Table 5 compared to the levels of the same RNA transcripts in a mesenchymal stem cell (MSC)-like cell deposited under ECACC accession number 22072103; and/or
(b) have reduced levels of at least 50 different RNA transcripts (e.g. 50 different RNA transcripts) selected from Table 6 compared to the levels of the same RNA transcripts in a mesenchymal stem cell (MSC)-like cell deposited under ECACC accession number 22072103.

The level (e.g. amount) of an RNA transcript in a cell may readily be determined by the skilled person using routine methods known in the art, for example qPCR. Furthermore, a person of skill in the art would readily be able to determine whether the derivative has increased levels of a particular RNA transcript (e.g. selected from Table 5) compared to the levels of the same RNA transcripts in a mesenchymal stem cell (MSC)-like cell deposited under ECACC accession number 22072103 and/or reduced levels of a particular RNA transcript (e.g. selected from Table 6) compared to the levels of the same RNA transcripts in a mesenchymal stem cell (MSC)-like cell deposited under ECACC accession number 22072103, for example, using routine methods known in the art. For example, the skilled person may employ the methods described in the examples section below (e.g. RNA-seq).

In one non-limiting example, the level (e.g. amount) of an RNA transcript in a cell (e.g. a Y201 FGFR3 KO cell) compared to a reference cell (e.g. a Y201 WT cell) may be determined as shown in the examples section below. As is described below, it may be concluded that there is an increase in the level of a particular RNA transcript when there is at least a log 2 fold increase of >1 and an adjusted p value of <0.05 in the RNA-seq data for that particular RNA transcript in the cell (e.g. the Y201 FGFR3 KO cell) compared to the reference cell (e.g. the Y201 WT cell). In some examples, RNA transcripts having <5FPKM reads in both cell lines (e.g. the Y201 FGFR3 KO cell and the Y201 WT cell) are excluded from the analysis and are not identified as being upregulated or downregulated. See for example, the analysis undertaken below in the examples section in respect of the data in Table 5.

In one non-limiting example, the level (e.g. amount) of an RNA transcript in a cell (e.g. a Y201 FGFR3 KO cell) compared to a reference cell (e.g. a Y201 WT cell) may be determined as shown in the examples section below. As is described below, it may be concluded that there is a reduction in the level of a particular RNA transcript when there is at least a log 2 fold reduction of <−1 and an adjusted p value of <0.05 in the RNA-seq data for that particular RNA transcript in the cell (e.g. the Y201 FGFR3 KO cell) compared to the reference cell (e.g. the Y201 WT cell). In some examples, RNA transcripts having <5FPKM reads in both cell lines (e.g. the Y201 FGFR3 KO cell and the Y201 WT cell) are excluded from the analysis and are not identified as being upregulated or downregulated. See for example, the analysis undertaken below in the examples section in respect of the data in Table 6.

The terms “increased”, “increase” or “up-regulated”, “higher” are all used herein to generally mean an increase by a statistically significant amount. For the avoidance of any doubt, the terms “increased” or “increase” means an increase compared to a reference level/control (e.g. the levels of RNA transcripts in a mesenchymal stem cell (MSC)-like cell deposited under ECACC accession number 22072103). For the avoidance of any doubt, in the context of the present invention, the level of an RNA transcript (e.g. an RNA transcript selected from Table 5 compared) may be increased by at least 10%, at least 20%, at least 30%, at least 40%, at least 50%, at least 60%, at least 70%, at least 80%, at least 90%, at least 100%, at least 110%, at least 120%, at least 130%, at least 140%, at least 150%, at least 160%, at least 170%, at least 180%, at least 190%, at least 200%, at least 210%, at least 220%, at least 230%, at least 240% or at least 250% as compared to the level of the same RNA transcript in a mesenchymal stem cell (MSC)-like cell deposited under ECACC accession number 22072103. For example, the level of an RNA transcript (e.g. an RNA transcript selected from Table 5 compared) may be increased by 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240% or 250% as compared to the level of the same RNA transcript in a mesenchymal stem cell (MSC)-like cell deposited under ECACC accession number 22072103. For example, the level of an RNA transcript (e.g. an RNA transcript selected from Table 5 compared) may be increased by at least about a 0.5-fold, or at least about a 1.0-fold, or at least about a 1.2-fold, or at least about a 1.5-fold, or at least about a 2-fold, or at least about a 3-fold, or at least about a 4-fold, or at least about a 5-fold or at least about a 10-fold increase, or any increase between 1.0-fold and 10-fold or greater as compared to the level of the same RNA transcript in a mesenchymal stem cell (MSC)-like cell deposited under ECACC accession number 22072103.

The terms “decrease”, “decreased” “reduced”, “reduction” or ‘down-regulated”, “lower” are all used herein generally to mean a decrease by a statistically significant amount. However, for avoidance of doubt, “reduced”, “reduction”, “decreased” or “decrease” means a decrease as compared to a reference level/control (e.g. the levels of the same RNA transcripts in a mesenchymal stem cell (MSC)-like cell deposited under ECACC accession number 22072103). For the avoidance of any doubt, in the context of the present invention, the level of an RNA transcript (e.g. an RNA transcript selected from Table 5 compared) may be reduced by at least 10%, at least 20%, at least 30%, at least 40%, at least 50%, at least 60%, at least 70%, at least 80%, at least 90%, at least 100%, at least 110%, at least 120%, at least 130%, at least 140%, at least 150%, at least 160%, at least 170%, at least 180%, at least 190%, at least 200%, at least 210%, at least 220%, at least 230%, at least 240% or at least 250% as compared to the level of the same RNA transcript in a mesenchymal stem cell (MSC)-like cell deposited under ECACC accession number 22072103. In some examples, the level of an RNA transcript (e.g. an RNA transcript selected from Table 5 compared) may be reduced by 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240% or 250% as compared to the level of the same RNA transcript in a mesenchymal stem cell (MSC)-like cell deposited under ECACC accession number 22072103. For example, the level of an RNA transcript (e.g. an RNA transcript selected from Table 6 compared) may be reduced by at least about a 0.5-fold, or at least about a 1.0-fold, or at least about a 1.2-fold, or at least about a 1.5-fold, or at least about a 2-fold, or at least about a 3-fold, or at least about a 4-fold, or at least about a 5-fold or at least about a 10-fold increase, or any increase between 1.0-fold and 10-fold or greater as compared to the level of the same RNA transcript in a mesenchymal stem cell (MSC)-like cell deposited under ECACC accession number 22072103.

The term “RNA transcript” is used herein in accordance with its conventional meaning, namely that refers to a single-stranded ribonucleic acid (RNA) that has been synthesized by transcription of DNA. RNA transcripts include mature RNA products such as mRNAs, tRNAs, and rRNAs, as well as precursor RNAs, such as precursor mRNAs (pre-mRNA) that become messenger RNA (mRNA) after processing.

As mentioned above, the inventors modified Y201 cells using CRISPR/Cas9 to target a deletion of the FGFR3 gene. Accordingly, a mesenchymal stem cell (MSC)-like cell deposited under ECACC accession number 22072101 or a derivative thereof is also provided herein. For the avoidance of doubt, the definition of “derivative” as provided above applies equally here.

A mesenchymal stem cell (MSC)-like cell deposited under ECACC accession number 22072101 may be referred to herein as a “Y201 FGFR3 KO cell” and/or a “FGFR3 KO MSC”. A FGFR3 KO MSC (deposited under ECACC accession number 22072101) was deposited at the UK Health Security Agency Porton Down and European Collection of Authenticated Cell Cultures, Porton Down, Salisbury, SP4 OJG, UK under The Budapest Treaty of 1977 on 21 Jul. 2022.

The inventors have surprisingly shown that the cells provided herein have tissue forming and anti-inflammatory properties. These cells can therefore be useful in e.g. a therapeutic setting.

Accordingly, a cell population comprising a plurality of cells as provided herein is also provided.

A “cell population” as used herein refers to a plurality of cells (i.e. 2 or more cells). Accordingly, as would be clear to the skilled person, in the context of the present invention “plurality” refers to “2 or more” (e.g. 2 or more, 3 or more, 4 or more, 5 or more, 6 or more, 7 or more, 8 or more, 9 or more, 10 or more, 20 or more, 40 or more, 60 or more, 80 or more, 100 or more etc.)

The cell population provided herein may be genetically homogenous (i.e. have substantially the same genotype) or be genetically heterogenous (i.e. have genetic differences, which can be natural genetic variations or man-made mutations, caused for example by mutagenesis and gene editing techniques).

As would be clear to the skilled person, the cell population may comprise any of the cells (e.g. a mesenchymal stem cell (MSC)-like cell deposited under ECACC accession number 22072103, a derivative thereof, a mesenchymal stem cell (MSC)-like cell deposited under ECACC accession number 22072101 and/or a derivative thereof) described herein.

In some examples, the cell population provided herein may comprise a plurality of mesenchymal stem cell (MSC)-like cells deposited under ECACC accession number 22072103.

In some examples, the cell population provided herein may comprise a plurality of derivatives of mesenchymal stem cell (MSC)-like cells deposited under ECACC accession number 22072103. For example, the cell population may comprise derivatives (e.g. of mesenchymal stem cell (MSC)-like cells deposited under ECACC accession number 22072103) having reduced FGFR3 expression and/or activity compared to a MSC-like cell deposited under ECACC accession number 22072103. For example, the cell population may comprise derivatives (e.g. of mesenchymal stem cell (MSC)-like cells deposited under ECACC accession number 22072103) wherein the derivative only differs from a MSC-like cell deposited under ECACC accession number 22072103 by having reduced FGFR3 expression and/or activity.

In some examples, the cell population provided herein may comprise a plurality of cells, wherein the plurality of cells is made up from (a) at least one mesenchymal stem cell (MSC)-like cell deposited under ECACC accession number 22072103 and (b) at least one derivative of a mesenchymal stem cell (MSC)-like cell deposited under ECACC accession number 22072103 (e.g, wherein the derivative has reduced FGFR3 expression and/or activity compared to a MSC-like cell deposited under ECACC accession number 22072103 or wherein the derivative only differs from a MSC-like cell deposited under ECACC accession number 22072103 by having reduced FGFR3 expression and/or activity).

In another example, the cell population provided herein may comprise a plurality of mesenchymal stem cell (MSC)-like cells deposited under ECACC accession number 22072101.

In another example, the cell population provided herein may comprise a plurality of derivatives of mesenchymal stem cell (MSC)-like cells deposited under ECACC accession number 22072101.

In another example, the cell population provided herein may comprise a plurality of cells, wherein the plurality of cells is made up from (a) at least one mesenchymal stem cell (MSC)-like cell deposited under ECACC accession number 22072101 and (b) at least one derivative of a mesenchymal stem cell (MSC)-like cell deposited under ECACC accession number 22072101.

In some examples, the cell population provided herein may comprise a plurality of cells, wherein the plurality of cells is made up from (a) at least one mesenchymal stem cell (MSC)-like cell deposited under ECACC accession number 22072103 and (b) at least one mesenchymal stem cell (MSC)-like cell deposited under ECACC accession number 22072101.

The cell populations described herein may therefore be a mixed population, comprising at least two genetically distinct cell sub-populations described herein. The mixed population may comprise a combination of any two or more cell types described in detail elsewhere herein.

A person of skill in the art is aware of how to generate a cell population (e.g. mixed cell population) as described herein, for example, using routine cell culture techniques. Cell culture is described in detail elsewhere herein.

The cells described herein (e.g. mesenchymal stem cell (MSC)-like cells deposited under ECACC accession number 22072103, derivatives thereof, mesenchymal stem cell (MSC)-like cells deposited under ECACC accession number 22072101 and/or derivatives thereof) may be used to obtain a preparation of genomic DNA by a DNA extraction method. The invention therefore further provides a DNA preparation comprising the genomic DNA of (e.g. extracted from) any of the cells described herein.

As would be clear to the skilled person, any of the cells described herein can be subject to genomic DNA extraction, e.g. through methods of DNA extraction well-known in the art, e.g. using organic extraction, silica spin columns, and magnetic beads. Therefore, the invention further provides for a DNA preparation comprising the genomic DNA extracted from any of the cells described herein.

In some examples, the DNA preparation provided herein comprises the genomic DNA of a mesenchymal stem cell (MSC)-like cell deposited under ECACC accession number 22072103. In another example, the DNA preparation provided herein comprises the genomic DNA of a derivative of a mesenchymal stem cell (MSC)-like cell deposited under ECACC accession number 22072103 (e.g, wherein the derivative has reduced FGFR3 expression and/or activity compared to a MSC-like cell deposited under ECACC accession number 22072103 or wherein the derivative only differs from a MSC-like cell deposited under ECACC accession number 22072103 by having reduced FGFR3 expression and/or activity).

In another example, the DNA preparation provided herein comprises the genomic DNA of a mesenchymal stem cell (MSC)-like cell deposited under ECACC accession number 22072101. In another example, the DNA preparation provided herein comprises the genomic DNA of a derivative of a mesenchymal stem cell (MSC)-like cell deposited under ECACC accession number 22072101.

“DNA preparation” as used herein refers to a sample comprising genomic DNA that has been obtained or generated from a cell described herein, typically wherein the genomic DNA is admixed with a DNA-compatible buffer. Suitable buffers are well known in the art, e.g. a buffer with a pH of about 7.5 to 8.0, such as a Tris-EDTA (TE) buffer. “Genomic DNA (gDNA)” as used herein refers to chromosomal DNA (e.g. the chromosomal DNA of a cell described herein).

As exemplified herein, the inventors have shown that cell-free conditioned media, the secretome and/or EVs obtained from the culture of Y201 cells or Y201 FGFR3 KO cells have advantageous therapeutic effects. A cell-free conditioned medium that is obtainable by cell culture of a cell described herein, or a cell population described herein is thus provided.

For example, a cell-free conditioned medium that is obtainable by cell culture of a mesenchymal stem cell (MSC)-like cell deposited under ECACC accession number 22072103 is provided herein.

For example, a cell-free conditioned medium that is obtainable by cell culture of a cell population comprising a plurality of mesenchymal stem cell (MSC)-like cells deposited under ECACC accession number 22072103 is also provided herein.

A cell-free conditioned medium that is obtainable by cell culture of a derivative of a mesenchymal stem cell (MSC)-like cell deposited under ECACC accession number 22072103 (e.g. a derivative of a mesenchymal stem cell (MSC)-like cell deposited under ECACC accession number 22072103 which has reduced FGFR3 expression and/or activity compared to a MSC-like cell deposited under ECACC accession number 22072103 and/or a derivative of a mesenchymal stem cell (MSC)-like cell deposited under ECACC accession number 22072103 that only differs from a MSC-like cell deposited under ECACC accession number 22072103 by having reduced FGFR3 expression and/or activity) is also provided herein.

For example, a cell-free conditioned medium that is obtainable by cell culture of a cell population comprising a plurality of derivatives of mesenchymal stem cell (MSC)-like cells deposited under ECACC accession number 22072103 is also provided. The cell population may comprise derivatives (e.g. of mesenchymal stem cell (MSC)-like cells deposited under ECACC accession number 22072103) having reduced FGFR3 expression and/or activity compared to a MSC-like cell deposited under ECACC accession number 22072103. For example, the cell population may comprise derivatives (e.g. of mesenchymal stem cell (MSC)-like cells deposited under ECACC accession number 22072103) wherein the derivative only differs from a MSC-like cell deposited under ECACC accession number 22072103 by having reduced FGFR3 expression and/or activity.

In another example, a cell-free conditioned medium that is obtainable by cell culture of a cell population comprising a plurality of cells, wherein the plurality of cells is made up from (a) at least one mesenchymal stem cell (MSC)-like cell deposited under ECACC accession number 22072103 and (b) at least one derivative of a mesenchymal stem cell (MSC)-like cell deposited under ECACC accession number 22072103 (e.g, wherein the derivative has reduced FGFR3 expression and/or activity compared to a MSC-like cell deposited under ECACC accession number 22072103 and/or wherein the derivative only differs from a MSC-like cell deposited under ECACC accession number 22072103 by having reduced FGFR3 expression and/or activity) is provided.

In another example, a cell-free conditioned medium that is obtainable by cell culture of a mesenchymal stem cell (MSC)-like cell deposited under ECACC accession number 22072101 is provided.

In another example, a cell-free conditioned medium that is obtainable by cell culture of a cell population comprising a plurality of mesenchymal stem cell (MSC)-like cells deposited under ECACC accession number 22072101 is also provided.

For example, a cell-free conditioned medium that is obtainable by cell culture of a derivative of a mesenchymal stem cell (MSC)-like cell deposited under ECACC accession number 22072101 is also provided.

For example, also provided is a cell-free conditioned medium that is obtainable by cell culture of a cell population comprising a plurality of derivatives of mesenchymal stem cell (MSC)-like cells deposited under ECACC accession number 22072101.

In another example, a cell-free conditioned medium that is obtainable by cell culture of a cell population comprising a plurality of cells, wherein the plurality of cells is made up from (a) at least one mesenchymal stem cell (MSC)-like cell deposited under ECACC accession number 22072101 and (b) at least one derivative of a mesenchymal stem cell (MSC)-like cell deposited under ECACC accession number 22072101, is also provided.

In some examples, a cell-free conditioned medium that is obtainable by cell culture of a cell population comprising a plurality of cells, wherein the plurality of cells is made up from (a) at least one mesenchymal stem cell (MSC)-like cell deposited under ECACC accession number 22072103 and (b) at least one mesenchymal stem cell (MSC)-like cell deposited under ECACC accession number 22072101, is also provided.

The synonymous phrases “cell-free” and “free of cells” are generally well-understood in the art, and, in the present context, may particularly signify that a composition (e.g. a conditioned medium or composition described herein) essentially does not contain cells (e.g. mesenchymal stem cell (MSC)-like cells deposited under ECACC accession number 22072103, derivatives thereof, mesenchymal stem cell (MSC)-like cells deposited under ECACC accession number 22072101 and/or derivatives thereof). In particular, “cell-free” and “free of cells” in the present context, may particularly signify that a composition (e.g. a conditioned medium or composition described herein) essentially does not contain viable cells (e.g. viable mesenchymal stem cell (MSC)-like cells deposited under ECACC accession number 22072103, viable derivatives thereof, viable mesenchymal stem cell (MSC)-like cells deposited under ECACC accession number 22072101 and/or viable derivatives thereof). The degree to which a composition (including a conditioned medium) is free of cells tends to be largely determined by the effectiveness of available methods for separating cells from culture media, such as, for example, centrifugation or filtration, or repetitions and/or combinations of such methods. For practical purposes, a composition, including a conditioned medium, may be considered cell-free when it contains 5×10² or fewer cells/ml, 100 or fewer cells/ml, 50 or fewer cells/ml, 25 or fewer cells/ml, 10 or fewer cells/ml, or 5 or fewer cells/ml, or no (i.e., 0) cells/ml; preferably these counts denote viable cells. Conventional cell counting methods may be used to determine the number of cells present in a composition (such as a conditioned medium), such as light microscopy, or flow cytometry, or plating and colony forming units (CFU) determination. Methods of determining cell viability are known to persons skilled in the art, for example conventional cell viability determination methods may be used, such as dye (e.g., trypan blue or propidium iodide) exclusion assays.

The cell-free conditioned media provided herein are obtainable by cell culture of a cell described herein, or cell culture of a cell population described herein. As used herein, the terms “culture”, and “cell culture” are common in the art and are used interchangeably herein. As used herein, the terms “culture”, and “cell culture” refer to the process whereby cells (e.g. a cell), (e.g. mesenchymal stem cell (MSC)-like cells deposited under ECACC accession number 22072103, derivatives thereof, mesenchymal stem cell (MSC)-like cells deposited under ECACC accession number 22072101 and/or derivatives thereof), are maintained and/or grown (e.g. divide) under controlled conditions, preferably in vitro or ex vivo.

Cell culture typically requires a cell culture medium. The term “medium” (or “media”) as used herein broadly encompasses any cell culture medium conducive to maintenance and/or proliferation of cells (e.g. mesenchymal stem cell (MSC)-like cells deposited under ECACC accession number 22072103, derivatives thereof, mesenchymal stem cell (MSC)-like cells deposited under ECACC accession number 22072101 and/or derivatives thereof, as described herein). Typically, the medium will be a liquid culture medium, which facilitates easy manipulation (e.g., decantation, pipetting, centrifugation, filtration, and such) thereof. As used herein the terms “medium”, “culture medium”, “culture media” and “media” are used interchangeably.

Various cell culture media will be known to those skilled in the art, who will also appreciate that the type of cells to be cultured may dictate the type of culture medium to be used. Preferably, the cells described herein are cultured in a defined culture media containing the minimum essential elements necessary to maintain the cells, wherein the components of the media are both known and controlled. Such minimum essential elements (and the corresponding defined culture media) for MSC culture (and accordingly the cells provided herein) are known in the art. For example, the culture conditions used herein (e.g. for Y201 cells and/or Y201 FGFR3 KO cells) may comprise culturing the cells in 5% CO₂ at 37 degrees Celsius. Examples of suitable cell culture media for use in accordance with the present invention are provided in the Examples section below. For example, serum-free medium and/or the presence or absence of specific nutrients may be beneficial when generating a cell conditioned medium as described elsewhere herein (for example, to keep the conditioned media described herein substantially free of contaminants).

Typically, the medium will comprise a basal medium formulation as known in the art. Many basal media formulations (available, e.g., from the American Type Culture Collection, ATCC; or from Invitrogen, Carlsbad, California) can be used to culture the cells described herein, including but not limited to Eagle's Minimum Essential Medium (MEM), Dulbecco's Modified Eagle's Medium (DMEM), alpha modified Minimum Essential Medium (alpha-MEM), Basal Medium Essential (BME), Iscove's Modified Dulbecco's Medium (IMDM), BGJb medium, F-12 Nutrient Mixture (Ham), Leibovitz L-15, DMEM/F-12, Essential Modified Eagle's Medium (EMEM), RPMI-1640, Medium 199, Waymouth's MB 752/1 or Williams Medium E, and modifications and/or combinations thereof. Compositions of the above basal media are generally known in the art and it is within the skill of one in the art to modify or modulate concentrations of media and/or media supplements as necessary for the cells cultured. A particularly preferred basal medium, especially for culturing the cells described herein (e.g. mesenchymal stem cell (MSC)-like cells deposited under ECACC accession number 22072103, derivatives thereof, mesenchymal stem cell (MSC)-like cells deposited under ECACC accession number 22072101 and/or derivatives thereof), may be DMEM.

Such basal media formulations contain ingredients necessary for mammalian cell maintenance and/or proliferation, which are known per se. By means of illustration and not limitation, these ingredients may include inorganic salts (in particular salts containing Na, K, Mg, Ca, Cl, P and possibly Cu, Fe, Se and Zn), physiological buffers (e.g., HEPES, bicarbonate), nucleotides, nucleosides and/or nucleic acid bases, ribose, deoxyribose, amino acids, vitamins, antioxidants (e.g., glutathione) and sources of carbon (e.g., glucose, sodium pyruvate, sodium acetate), etc.

For use in culture, basal media can be supplied with one or more further components. For example, in some instances, additional supplements can be used to supply the cells with the necessary trace elements and substances for optimal maintenance, growth, and/or expansion. Furthermore, antioxidant supplements may be added at appropriate concentrations, e.g., β-mercaptoethanol or N-acetyl-L-cysteine. While many basal media already contain amino acids, some amino acids may be supplemented later, e.g., L-glutamine, which is known to be less stable when in solution. In further examples, a medium may be further supplied with antibiotic and/or antimycotic compounds, such as, typically, mixtures of penicillin and streptomycin, and/or other compounds, not limited to, amphotericin, ampicillin, gentamicin, bleomycin, hygromycin, kanamycin, mitomycin, mycophenolic acid, nalidixic acid, neomycin, nystatin, paromomycin, polymyxin, puromycin, rifampicin, spectinomycin, tetracycline, tylosin, and zeocin.

In some examples, media is supplemented with Penicillin/Streptomycin (P/S).

Lipids and lipid carriers can also be used to supplement cell culture media. Such lipids and carriers can include, but are not limited to cyclodextrin, cholesterol, linoleic acid conjugated to albumin, linoleic acid and oleic acid conjugated to albumin, unconjugated linoleic acid, linoleic-oleic-arachidonic acid conjugated to albumin, oleic acid unconjugated and conjugated to albumin, among others. Albumin can similarly be used in fatty-acid free formulations.

In some examples, cell culture media may be supplemented with mammalian plasma or sera. Plasma or sera often contain cellular factors and components that facilitate cell viability and expansion. Optionally, plasma or serum may be heat inactivated. Heat inactivation is used in the art mainly to remove the complement. Heat inactivation typically involves incubating the plasma or serum at 56 °C for 30 to 60 min, e.g., 30 min, with steady mixing, after which the plasma or serum is allowed to gradually cool to ambient temperature. A skilled person will be aware of any common modifications and requirements of the above procedure. Optionally, plasma or serum may be sterilised prior to storage or use. Usual means of sterilisation may involve, e.g., filtration through one or more filters with pore size smaller than 1 μm, preferably smaller than 0.5 μm, e.g., smaller than 0.45 μm, 0.40 μm, 0.35 μm, 0.30 μm or 0.25 μm, more preferably 0.2 μm or smaller, e.g., 0.15 μm or smaller, 0.10 μm or smaller. Suitable sera or plasmas for use in media as taught herein may include human serum or plasma, or serum or plasma from non-human animals, for example, non-human mammals, such as, e.g., non-human primates (e.g., lemurs, monkeys, apes), foetal or adult bovine, horse, porcine, lamb, goat, dog, rabbit, mouse or rat serum or plasma, etc, or any combination of such.

A medium as taught herein may preferably comprise bovine serum or plasma, preferably foetal bovine (calf) serum or plasma, more preferably foetal bovine (calf) serum (FCS or FBS).

In some examples, media is supplemented with FBS and Penicillin/Streptomycin (P/S).

In some examples, serum or plasma can be replaced (e.g. during cell culture) by serum replacements, such as to provide for serum-free media (i.e., chemically defined media). Serum-free media may therefore be used to culture the cells described herein. The provision of serum-free media may be advantageous particularly with view to administration of the media to subjects, especially to human subjects (e.g., improved bio-safety). “Serum replacement” as used herein broadly refers to any a composition that may be used to replace animal serum in a cell culture medium. For example, a serum replacement may replace the functions (e.g., the cell maintenance and growth supportive functions) of animal serum in a cell culture medium. A conventional serum replacement may typically comprise vitamins, albumin, lipids, amino acids, transferrin, antioxidants, insulin and trace elements. Many commercialized serum replacement additives, such as KnockOut Serum Replacement (KOSR), N2, B27, Insulin-Transferrin-Selenium Supplement (ITS), and G5 are well known and are readily available to those skilled in the art. For example, DMEM (e.g. DMEM supplemented with P/S) may be used.

Plasma or serum or serum replacement may be comprised in media as taught herein at any suitable proportion (volume of plasma or serum or serum replacement/volume of medium). For example, between about 0.5% v/v and about 70.0% v/v, preferably between about 10.0% v/v and about 60.0% v/v, e.g., between about 10.0% v/v and about 55.0% v/v. For example, plasma or serum or serum replacement may be comprised in media as taught herein between about 0.5% v/v and about 10% v/v. For example, plasma or serum or serum replacement may be comprised in media as taught herein at about 10.0% v/v.

In some examples, the medium (e.g. the conditioned medium described in more detail below) may lack any serum or plasma, i.e., the medium (e.g. conditioned medium) may be serum-free medium (e.g. a serum-free conditioned medium). The provision of these media can improve the bio-safety and/or immunological profile of the medium (e.g. the conditioned medium).

As an example, a medium for culturing a cell described herein may comprise Dulbecco's Modified Eagle's Medium (DMEM) (Cat: 41966, ThermoFisher Scientific) supplemented with 20% Foetal Bovine Serum (FBS) and 1% Penicillin/Streptomycin (P/S) (Cat: 15140122, ThermoFisher Scientific). In some examples, a medium for culturing the cells described herein may comprise Dulbecco's Modified Eagle's Medium (DMEM) (Cat: 41966, ThermoFisher Scientific) supplemented with 20% Foetal Bovine Serum (FBS) and 1% Penicillin/Streptomycin (P/S) (Cat: 15140122, ThermoFisher Scientific) hwerein the medium has been centrifuged (e.g. at 10,000 g for 18 hrs at 4 °C) to deplete the medium of serum-derived bovine EVs. In some examples, during cell expansion, this medium may be diluted in equal volumes with DMEM supplemented with 1% P/S.

A particularly preferred medium, especially for culturing Y201 WT cells, may be a serum-free medium.

A particularly preferred medium, especially for culturing Y201 FGFR3 KO cells, may comprise DMEM.

The term “conditioned medium” as used herein refers to a medium that has been exposed to (e.g. contacted with) at least one cell for a sufficient period of time such that the medium comprises at least one additional component that was not present in the medium before exposing the medium to the cell, wherein the at least one additional component is a cell product/metabolite. In other words, a “conditioned medium” may be deemed as a medium comprising cell secretion products, such as inter alia cell secretion proteins and cellular metabolites. The at least one cell may have been grown in culture, i.e. in an appropriate medium, for a sufficient period of time such that the conditioned medium comprises at least one additional component that was not present in the medium before exposing the medium to the cell, wherein the at least one additional component is a cell product/metabolite. Conditioned media may be referred to as “CM” herein.

The period of time sufficient for the medium exposed to (e.g. contacted with) at least one cell to comprise at least one additional component that was not present in the medium before exposing the medium to the cell (wherein the at least one additional component is/was produced by the cell) may, in particular, be a period of time sufficient for the at least one cell to achieve secretion of secretion products (including e.g. secreted proteins and extracellular vesicles) into the medium. By means of example, said period of time may be at least about 1 hour, at least about 3 hours, at least about 6 hours, at least about 12 hours, at least about 18 hours, at least about 24 hours, at least about 36 hours or at least about 48 hours. Typically, said period of time will be no more than about 72 hours, more typically no more than about 60 hours, even more typically no more than about 48 hours.

In some examples, the period of time sufficient for the medium exposed to (e.g. contacted with) at least one cell to comprise at least one additional component that was not present in the medium before exposing the medium to the cell (wherein the at least one additional component is/was produced by the cell) may be at least 24 hours. Accordingly, in some examples, the period of time sufficient for the medium exposed to (e.g. contacted with) at least one cell to comprise at least one additional component that was not present in the medium before exposing the medium to the cell (wherein the at least one additional component is/was produced by the cell) may be about 24 hours.

In some examples, the period of time sufficient for the medium exposed to (e.g. contacted with) at least one cell to comprise at least one additional component that was not present in the medium before exposing the medium to the cell (wherein the at least one additional component is/was produced by the cell) may be at least 48 hours. Accordingly, in some examples, the period of time sufficient for the medium exposed to (e.g. contacted with) at least one cell to comprise at least one additional component that was not present in the medium before exposing the medium to the cell (wherein the at least one additional component is/was produced by the cell) may be about 48 hours.

A conditioned medium may thus be obtainable by or directly obtained by culturing a cell provided herein or a cell population provided herein in a cell culture medium, thereby conditioning the medium. A cell-free conditioned medium may be obtained by separating the culture medium from said cell or cell population, thereby obtaining the cell-free conditioned medium as described elsewhere herein. The term “cell-free” is defined above and applies equally here. Thus, as would be clear to a person of skill in the art, a “cell-free conditioned medium” is a conditioned medium (whereby a conditioned medium is a conditioned medium as defined herein) that essentially does not contain cells (e.g. mesenchymal stem cell (MSC)-like cells deposited under ECACC accession number 22072103, derivatives thereof, mesenchymal stem cell (MSC)-like cells deposited under ECACC accession number 22072101 and/or derivatives thereof).

Typically, animal cells, such as mammalian cells, such as human cells, are cultured by exposing them to (i.e., contacting them with) a suitable cell culture medium in a vessel or container adequate for the purpose of an experiment (e.g., a 96-, 24-, or 6-well plate, a T-25, T-75, T-150, T-175 or T-225 flask, or a cell factory), at art-known conditions conducive to in vitro cell culture, such as temperature of 37 °C, 5% v/v CO2 and >95% humidity. In some examples, the cells (e.g. mesenchymal stem cell (MSC)-like cells deposited under ECACC accession number 22072103, derivatives thereof, mesenchymal stem cell (MSC)-like cells deposited under ECACC accession number 22072101 and/or derivatives thereof) and/or cell populations described herein are cultured in T175 flasks.

Regarding cell culture conditions, appropriate material for cell culture (e.g., plates, flasks, or bioreactors) can readily be selected by a person skilled in the art. Other cell culture conditions that can be finely adapted for obtaining a conditioned medium described herein having the desired composition and properties include temperature, cell density at seeding, and oxygen tension.

When preparing a conditioned medium, cells (e.g. mesenchymal stem cell (MSC)-like cells deposited under ECACC accession number 22072103, derivatives thereof, mesenchymal stem cell (MSC)-like cells deposited under ECACC accession number 22072101 and/or derivatives thereof) by the action of which the medium is to be conditioned may be contacted with the medium at variety of initial cell densities. By means of example the cells described herein (e.g. mesenchymal stem cell (MSC)-like cells deposited under ECACC accession number 22072103, derivatives thereof, mesenchymal stem cell (MSC)-like cells deposited under ECACC accession number 22072101 and/or derivatives thereof) or population of cells may be contacted with the medium at initial cell confluence of at least about 50%, e.g., at least about 55%, at least about 60%, e.g., at least about 65%, or at least about 70%, at least about 75%, or at least about 80%, e.g., at least about 85%, or of least about 90%, e.g., at least about 95%, such as 96%, 97%, 98%, 99%, or even 100% initial cell confluence.

The term “confluence” refers to density of cultured cells in which the cells contact one another covering substantially all of the surfaces available for cell proliferation (i.e., fully confluent).

In some examples, the cells (e.g. mesenchymal stem cell (MSC)-like cells deposited under ECACC accession number 22072103, derivatives thereof, mesenchymal stem cell (MSC)-like cells deposited under ECACC accession number 22072101 and/or derivatives thereof) and/or cell populations described herein may be contacted with the medium at initial cell confluence of at least about 80%. In another example, the cells described herein (e.g. mesenchymal stem cell (MSC)-like cells deposited under ECACC accession number 22072103, derivatives thereof, mesenchymal stem cell (MSC)-like cells deposited under ECACC accession number 22072101 and/or derivatives thereof) may be contacted with the medium at initial cell confluence of at least about 90%.

By means of example, the cells (e.g. mesenchymal stem cell (MSC)-like cells deposited under ECACC accession number 22072103, derivatives thereof, mesenchymal stem cell (MSC)-like cells deposited under ECACC accession number 22072101 and/or derivatives thereof) and/or cell populations described herein may be contacted with the medium at an initial cell density of at least about 1000 cells/cm² growth surface area, e.g., at least about 2000 cells/cm², at least about 10,000 cells/cm², at least about 25,000 cells/cm², at least about 50,000 cells/cm² growth surface area, e.g., at least about 75,000 cells/cm², at least about 85,000 cells/cm² initial cell density. For example, the cells described herein may be contacted with the medium at initial cell density of no more than about 100,000 cells/cm², typically no more than about 95,000 cells/cm², more typically no more than about 90,000 cells/cm² initial cell density. Hence, by means of example the cells described herein may usually be contacted with the medium at initial cell density of between about 1000 cells/cm² and about 100,000 cells/cm², such as, e.g., between about 2,000 cells/cm² and about 100,000 cells/cm², such as, e.g., about 80,000 cells/cm².

When preparing a conditioned medium, the medium to be conditioned may be provided at volumes commonplace in tissue culture. Typically, the cells described herein may be contacted with between about 0.10 mL/cm² growth surface area and about 0.20 mL/cm² growth surface area of medium, more typically between about 0.12 mL/cm² and about 0.18 mL/cm², even more typically between about 0.13 mL/cm² and about 0.16 mL/cm². Typically, the cells described herein may be contacted with about 0.14 mL/cm² growth surface area of medium.

A method for producing a cell-free conditioned medium may comprise the steps of culturing a cell or a cell population described herein in a cell culture medium and separating the culture medium from the cell(s). This method can be performed by using a cell culture medium that is a serum-free medium, by modifying specific conditions of cell culture, and/or by separating the cell culture medium from the cell(s) after culturing said cell(s) at given time points. In some examples, the culture medium is separated from the cells (e.g. mesenchymal stem cell (MSC)-like cells deposited under ECACC accession number 22072103, derivatives thereof, mesenchymal stem cell (MSC)-like cells deposited under ECACC accession number 22072101 and/or derivatives thereof) or cell population described herein after at least about 1 hour, at least about 3 hours, at least about 6 hours, at least about 12 hours, at least about 18 hours, at least about 24 hours, at least about 36 hours or at least about 48 hours of exposure to (e.g. contact with) the cell or population of cells. Typically, said period of time will be no more than about 72 hours, more typically no more than about 60 hours, even more typically no more than about 48 hours. As would be clear to the skilled person, relevant time points can be very short (e.g. 2 hours or less) or longer such as at 24 hours, at 36 hours or more hours or intermediate ones (such as 1 hour, 2 hours, 4 hours, 6 hours, 8 hours, 12 hours, or 18 hours).

In some examples, the culture medium is separated from the cells (e.g. mesenchymal stem cell (MSC)-like cells deposited under ECACC accession number 22072103, derivatives thereof, mesenchymal stem cell (MSC)-like cells deposited under ECACC accession number 22072101 and/or derivatives thereof) or cell population described herein after at least about 24 hours of exposure to (e.g. contact with) the cell or population of cells described herein.

In some examples, the culture medium is separated from the cells (e.g. mesenchymal stem cell (MSC)-like cells deposited under ECACC accession number 22072103, derivatives thereof, mesenchymal stem cell (MSC)-like cells deposited under ECACC accession number 22072101 and/or derivatives thereof) or cell population described herein after at least about 48 hours of exposure to (e.g. contact with) the cell or population of cells described herein.

In some examples, the cells (e.g. mesenchymal stem cell (MSC)-like cells deposited under ECACC accession number 22072103, derivatives thereof, mesenchymal stem cell (MSC)-like cells deposited under ECACC accession number 22072101 and/or derivatives thereof) or cell populations described herein may be used to prepare (e.g. to generate) one or more (e.g. two) discrete cell-free conditioned media. As discussed above, a method for producing a cell-free conditioned medium may comprise the steps of culturing a cell or a cell population described herein in a cell culture medium and separating the culture medium from the cell(s). This method can be performed by using a cell culture medium that is a serum-free medium, by modifying specific conditions of cell culture, and/or by separating the cell culture medium from the cell(s) after culturing said cell(s) at given time points. In some examples, a first culture medium may be separated from the cells (e.g. mesenchymal stem cell (MSC)-like cells deposited under ECACC accession number 22072103, derivatives thereof, mesenchymal stem cell (MSC)-like cells deposited under ECACC accession number 22072101 and/or derivatives thereof) or cell population described herein after at least about 24 hours of exposure to (e.g. contact with) the cell or population of cells described herein. Cell culture media may be then be replenished and a second culture medium may be separated from the cells (e.g. mesenchymal stem cell (MSC)-like cells deposited under ECACC accession number 22072103, derivatives thereof, mesenchymal stem cell (MSC)-like cells deposited under ECACC accession number 22072101 and/or derivatives thereof) or cell population used to prepare the first culture medium after a further at least about 24 hours of exposure to (e.g. contact with) the cell or population of cells. In other words, the cells (e.g. mesenchymal stem cell (MSC)-like cells deposited under ECACC accession number 22072103, derivatives thereof, mesenchymal stem cell (MSC)-like cells deposited under ECACC accession number 22072101 and/or derivatives thereof) or cell populations described herein may be used to prepare a first and a second culture media, wherein the first culture medium may be separated from the cells or cell population described herein after at least about 24 hours of exposure to (e.g. contact with) the cell or population of cells, and the second culture media may be separated from the cells or cell population described herein after a further at least about 24 hours of exposure to (e.g. contact with) the cell or population of cells. In some examples, the first culture medium and second culture medium may be pooled (e.g. after the first culture medium and the second culture medium has been separated from the cells or population of cells described herein).

In some examples, a first culture medium may be separated from the cells (e.g. mesenchymal stem cell (MSC)-like cells deposited under ECACC accession number 22072103, derivatives thereof, mesenchymal stem cell (MSC)-like cells deposited under ECACC accession number 22072101 and/or derivatives thereof) or cell population described herein after about 24 hours of exposure to (e.g. contact with) the cell or population of cells described herein. Cell culture media may be then be replenished and a second culture medium may be separated from the cells (e.g. mesenchymal stem cell (MSC)-like cells deposited under ECACC accession number 22072103, derivatives thereof, mesenchymal stem cell (MSC)-like cells deposited under ECACC accession number 22072101 and/or derivatives thereof) or cell population used to prepare the first culture medium after a further about 24 hours of exposure to (e.g. contact with) the cell or population of cells.

As would be known to a person of skill in the art, a culture medium may be separated from the cells (e.g. mesenchymal stem cell (MSC)-like cells deposited under ECACC accession number 22072103, derivatives thereof, mesenchymal stem cell (MSC)-like cells deposited under ECACC accession number 22072101 and/or derivatives thereof) or cell population described herein when the cells or cell population is at any appropriate confluence (e.g. to obtain a cell-free conditioned medium). For example, a culture medium may be separated from the cells (e.g. mesenchymal stem cell (MSC)-like cells deposited under ECACC accession number 22072103, derivatives thereof, mesenchymal stem cell (MSC)-like cells deposited under ECACC accession number 22072101 and/or derivatives thereof) or cell population described herein when the cells or cell population is at a confluence of about 80% to about 90%. In some examples, 80% confluency may equate to about 24000 cells/cm² for Y201 cells.

A conditioned medium may be separated from the cell or population of cells used to condition the medium by any available technique. Conventional techniques include for example removal of the medium from a culture vessel by decantation or pipetting, centrifugation of the medium to pellet cells, cell fragments and particulates present therein (e.g., centrifugation at between about 100 g and about 2,000 g, such as between about 200 g and about 1,500 g, such as at about 300 g, for between about 3 min and about 30 min, such as between about 5 min and 20 min, such as for about 5 min), filtration of the medium to filter away cells, cell fragments and particulates present therein (e.g., filtration through a standard microbiological filter having pore size about 1.0 μm or less, about 0.8 μm or less, about 0.6 μm or less, about 0.4 μm or less, such as about 0.2 μm). It shall be appreciated that repetitions and/or combinations of such methods may be employed to attain comparatively more complete separation. Examples of techniques to separate a conditioned medium from the cell or population of cells used to condition the medium are well known in the art and specific examples are provided in the Examples section below.

In some examples, the separation of conditioned media from a cell or cell population described herein may be performed by simply transferring the supernatant of the cell culture container or cell population culture container (by decantation or pipetting) into a separate container and, optionally, repeating and/or combining filtration (e.g., filtration through a standard microbiological filter having pore size about 1.0 μm or less, about 0.8 μm or less, about 0.6 μm or less, about 0.4 μm or less, such as about 0.2 μm) or centrifuging this cell culture supernatant at low speed (e.g., centrifugation at between about 100 g and about 2000 g, such as between about 200 g and about 1500 g, such as at about 300 g, for between about 3 min and about 30 min, such as between about 5 min and 20 min, such as for about 5 min), to pellet any remaining cell, cell debris, or particulate. In this manner, a cell-free preparation is obtained (the supernatant of the centrifugation) and it can be then used for determining identity and concentration of the biological molecules that are present, such as soluble proteins or extracellular vesicles as defined below, and according to commonly available technology such as immunoassays, spectrometry (e.g. LC-MS) or enzymatic assays.

In a particular example, the separation of conditioned media from a cell or cell population described herein may be performed by transferring the supernatant of the cell culture container or cell population culture container (by decantation or pipetting) into a separate container and, optionally, centrifuging this cell culture supernatant at low speed (e.g., centrifugation at 300 g for about 5 min), to pellet any remaining cell, cell debris, or particulate. The resultant supernatant after centrifugation may be referred to as cell-free conditioned media herein.

In a particular example, the separation of conditioned media from a cell or cell population described herein may be performed by centrifuging the cell culture or cell population culture at low speed (e.g., centrifugation at 300 g for about 5 min), to pellet any remaining cell, cell debris, or particulate. The resultant supernatant after centrifugation may be referred to as cell-free conditioned media herein.

The conditioned media described herein may comprise any components secreted by the cell or cell population used to condition the medium. Typically, the conditioned media described herein comprise soluble proteins and extracellular vesicles secreted by the cell or cell population used to condition the medium.

In a particular example, the conditioned medium described herein may comprise a secretome of a cell (or a secretome of a population of cells) used to condition the medium. Accordingly, a secretome, or a portion thereof, that is obtainable by cell culture of a cell (e.g. a mesenchymal stem cell (MSC)-like cell deposited under ECACC accession number 22072103, a derivative thereof, a mesenchymal stem cell (MSC)-like cell deposited under ECACC accession number 22072101 and/or a derivative thereof) described herein, or a cell population described herein is therefore provided.

As used herein, “secretome” refers to the totality (or collection) of components secreted or released by a cell or a cell population to the surroundings of the cell or cell population (e.g. into culture medium) when the cell or cell population is cultured. Components of a secretome may be organic and/or inorganic. Typically the components of a secretome include proteins and extracellular vesicles (EVs) (including exosomes and microvesicles). Thus, typically, the secretomes described herein comprise soluble proteins and EVs secreted by the cell or cell population used to condition the medium. The components of a secretome may also comprise lipids, cytokines (cell messengers), hormone-like substances and so-called immunomodulatory substances. ECM components are also typically components of the secretome thus, as would be clear to the skilled person, ECM components are typically present in conditioned media.

A secretome, or a portion thereof, that is obtainable by cell culture of a mesenchymal stem cell (MSC)-like cell deposited under ECACC accession number 22072103 is provided herein.

A secretome, or a portion thereof, that is obtainable by cell culture of a cell population comprising a plurality of mesenchymal stem cell (MSC)-like cells deposited under ECACC accession number 22072103 is also provided herein.

A secretome, or a portion thereof, that is obtainable by cell culture of a derivative of a mesenchymal stem cell (MSC)-like cell deposited under ECACC accession number 22072103 (e.g. a derivative of a mesenchymal stem cell (MSC)-like cell deposited under ECACC accession number 22072103 which has reduced FGFR3 expression and/or activity compared to a MSC-like cell deposited under ECACC accession number 22072103 or a derivative of a mesenchymal stem cell (MSC)-like cell deposited under ECACC accession number 22072103 that only differs from a MSC-like cell deposited under ECACC accession number 22072103 by having reduced FGFR3 expression and/or activity) is also provided herein.

A secretome, or a portion thereof, that is obtainable by cell culture of a cell population comprising a plurality of derivatives of mesenchymal stem cell (MSC)-like cells deposited under ECACC accession number 22072103 is also provided. The cell population may comprise derivatives (e.g. of mesenchymal stem cell (MSC)-like cells deposited under ECACC accession number 22072103) having reduced FGFR3 expression and/or activity compared to a MSC-like cell deposited under ECACC accession number 22072103. For example, the cell population may comprise derivatives (e.g. of mesenchymal stem cell (MSC)-like cells deposited under ECACC accession number 22072103) wherein the derivative only differs from a MSC-like cell deposited under ECACC accession number 22072103 by having reduced FGFR3 expression and/or activity.

In one example, a secretome, or a portion thereof, that is obtainable by cell culture of a cell population comprising a plurality of cells, wherein the plurality of cells is made up from (a) at least one mesenchymal stem cell (MSC)-like cell deposited under ECACC accession number 22072103 and (b) at least one derivative of a mesenchymal stem cell (MSC)-like cell deposited under ECACC accession number 22072103 (e.g, wherein the derivative has reduced FGFR3 expression and/or activity compared to a MSC-like cell deposited under ECACC accession number 22072103 and/or wherein the derivative only differs from a MSC-like cell deposited under ECACC accession number 22072103 by having reduced FGFR3 expression and/or activity) is provided.

A secretome, or a portion thereof, that is obtainable by cell culture of a mesenchymal stem cell (MSC)-like cell deposited under ECACC accession number 22072101 is further provided.

A secretome, or a portion thereof, that is obtainable by cell culture of a cell population comprising a plurality of mesenchymal stem cell (MSC)-like cells deposited under ECACC accession number 22072101 is also provided.

A secretome, or a portion thereof, that is obtainable by cell culture of a derivative of a mesenchymal stem cell (MSC)-like cell deposited under ECACC accession number 22072101 is also provided.

For example, also provided is a secretome, or a portion thereof, that is obtainable by cell culture of a cell population comprising a plurality of derivatives of mesenchymal stem cell (MSC)-like cells deposited under ECACC accession number 22072101.

In another example, a secretome, or a portion thereof, that is obtainable by cell culture of a cell population comprising a plurality of cells, wherein the plurality of cells is made up from (a) at least one mesenchymal stem cell (MSC)-like cell deposited under ECACC accession number 22072101 and (b) at least one derivative of a mesenchymal stem cell (MSC)-like cell deposited under ECACC accession number 22072101, is also provided.

In another example, a secretome, or a portion thereof, that is obtainable by cell culture of a cell population comprising a plurality of cells, wherein the plurality of cells is made up from (a) at least one mesenchymal stem cell (MSC)-like cell deposited under ECACC accession number 22072103 and (b) at least one mesenchymal stem cell (MSC)-like cell deposited under ECACC accession number 22072101, is also provided.

As used herein a “portion” of a secretome refers to a sample of the secretome, which may, for example, be isolated from the rest of the secretome. In one example, a portion of the secretome may be a particular fraction (isolated by e.g. size of the secretome components therein) of the secretome as a whole. In other words, the portion may be enriched in certain components of the secretome as a whole (wherein the portion has a higher proportion of certain components compared to the secretome as a whole). In this context, the term “fraction” refers to the result of a separation process, in which a mixture (e.g., a solid, liquid, solute or suspension) is divided up in, i.e., separated into, two or more smaller quantities (“fractions”) in which the composition changes. Hence, the composition of a fraction is altered compared to, i.e., is distinct from, the composition of the mixture subjected to the fractionation.

A secretome, or a portion thereof, may be obtained (e.g. from a cell cultured medium, as described herein) using routine methods known in the art. For example, the secretome may be obtained from a conditioned medium described herein, using methods such as centrifugation, filtration or dialysis.

In some examples, the cell-free conditioned medium provided herein may be serum-free.

As discussed elsewhere herein, the conditioned media and/or secretomes provided herein may comprise any components secreted by the cell or cell population used to condition the medium, such as extracellular vesicles (EVs). For certain purposes described herein, it may be advantageous to remove EVs from the conditioned media and/or secretomes described herein.

Accordingly, in some examples, the cell-free conditioned medium provided herein may be depleted of extracellular vesicles (EVs).

Similarly, in some examples, the secretome, or portion thereof, provided herein may be depleted of extracellular vesicles (EVs).

As would be clear to a skilled person, a conditioned medium or secretome provided herein that is depleted of extracellular vesicles (EVs) has a minimal amount of contaminating EVs. In other words, a conditioned medium or secretome provided herein depleted of EVs may be predominantly composed of components (e.g. cellular or extracellular proteins, lipids, carbohydrates, lipoproteins etc) that are not associated with EVs. In this context, a “minimal amount” may include less than 10% (by concentration) of EV contaminants, less than 5%, less than 2%, less than 1%, less than 0.5%, less than 0.25%, less than 0.1% etc (by concentration) of EV contaminants. The level of contamination does not need to be 0%. The level of contamination may be determined using EM.

The conditioned media or secretomes described herein sample may be substantially free of EVs. In a particular example, the cell-free conditioned medium provided herein may be substantially free of extracellular vesicles (EVs). Similarly, in a particular example, the secretome, or portion thereof, provided herein may be substantially free of extracellular vesicles (EVs).

The term “substantially free” when referring to a conditioned medium or secretome substantially free of EVs means the percentage of EVs in the conditioned medium or secretome (as appropriate) is significantly lower than that found in the starting conditioned medium or secretome (e.g. the unprocessed conditioned medium or secretome) from which the conditioned medium or secretome substantially free of EVs has been generated. Typically, the percentage of EVs in a conditioned medium or a secretome substantially free of EVs is less than 5%, 4%, 3%, 2% or 1% of the total conditioned medium or secretome, preferably wherein the percentage of EVs in a conditioned medium or a secretome substantially free of EVs is less than 0.5%, less than 0.25%, less than 0.1% etc of the total conditioned medium or secretome. The level of contamination does not need to be 0%.

EVs may be removed (e.g. extracted) from a conditioned medium or secretome provided herein by routine methods known in the art (Brennan et al, 2020, A comparison of methods for the isolation and separation of extracellular vesicles from protein and lipid particles in human serum. Sci Rep 10, 1039). In other words, a conditioned medium or secretome provided herein may be depleted of EVs by any suitable means. Examples of suitable methods are described in the Examples section below. Preferably, EVs are removed (e.g. extracted) from a conditioned medium or secretome described herein using (differential) ultra-centrifugation. Conditioned medium may be centrifuged at varying relative centrifugal force (rcf) to obtain (e.g. remove) EVs of corresponding densities. Typically, the vast majority of EVs can be obtained (and thus removed) by 10,000 g and 100,000 g centrifugation steps. Preferably, the fractions obtained by 2,000 g and 10,000 g centrifugation steps are removed from the conditioned medium, followed by collection (and thus removal) of the fraction obtained by centrifugation at 100,000 g.

In some examples, EVs are removed (e.g. extracted) from a conditioned medium or secretome described herein using tangential flow filtration (TFF).

Extracellular vesicles have been well characterised and have a well defined meaning in the art (reviewed in Andaloussi et al., Nature Reviews Drug Discovery, vol 12, May 2013, page 347-357). As used herein, “extracellular vesicle” refers to any membrane-bound particle secreted from a cell. Extracellular vesicles have been isolated from several bodily fluids. They have been shown to play a key role in the regulation of physiological processes, including stem cell maintenance, immune surveillance and blood coagulation. They have also been shown to play a crucial role in the pathology underlying several diseases.

Extracellular vesicles are released intact from cells and can either be shed from multivesicular bodies (MVBs), which are derived from endosomes, or bud directly out from the plasma membrane. When they are released (e.g. into the surrounding of a cell, e.g. into the extracellular space) they are referred to as exosomes or ectosomes (or microvesicles) depending on whether they have formed from inner or outer cell membranes. EVs may be taken up by other cells through endocytosis or fusion.

Extracellular vesicles are classified according to their cellular origin, biological function, or based on their biogenesis (reviewed in Andaloussi et al., 2013). As determined by their biogenesis, the three main classes of extracellular vesicles are exosomes, microvesicles and apoptotic bodies, the first two of which are most predominant in biological samples (and thus in EV samples derived therefrom). EV markers are well known in the art. Examples of exosome markers include tetraspanins (such as TSPAN29 and TSPAN30), ESCRT components, PDCD6IP, TSG101 and flotillin. Examples of microvesicle markers include integrins, selectins and CD40 ligand.

Despite recent advances, the terms “exosome” and “microvesicle” have been used interchangeably in many published studies. Herein, the term “extracellular vesicle” is used to refer to both vesicle types.

The inventors have also shown that the EVs obtained from cell culture of the cells or cell populations described herein have advantageous therapeutic properties.

Accordingly, also provided herein is an extracellular vesicle (EV) population that is obtainable by cell culture of a cell provided herein, or a cell population provided herein.

In one example, an extracellular vesicle (EV) population that is obtainable by cell culture of a mesenchymal stem cell (MSC)-like cell deposited under ECACC accession number 22072103 is provided herein.

An extracellular vesicle (EV) population that is obtainable by cell culture of a cell population comprising a plurality of mesenchymal stem cell (MSC)-like cells deposited under ECACC accession number 22072103 is also provided herein.

In another example, an extracellular vesicle (EV) population that is obtainable by cell culture of a derivative of a mesenchymal stem cell (MSC)-like cell deposited under ECACC accession number 22072103 (e.g. a derivative of a mesenchymal stem cell (MSC)-like cell deposited under ECACC accession number 22072103 which has reduced FGFR3 expression and/or activity compared to a MSC-like cell deposited under ECACC accession number 22072103 or a derivative of a mesenchymal stem cell (MSC)-like cell deposited under ECACC accession number 22072103 that only differs from a MSC-like cell deposited under ECACC accession number 22072103 by having reduced FGFR3 expression and/or activity) is also provided herein.

An extracellular vesicle (EV) population that is obtainable by cell culture of a cell population comprising a plurality of derivatives of mesenchymal stem cell (MSC)-like cells deposited under ECACC accession number 22072103 is also provided. The cell population may comprise derivatives (e.g. of mesenchymal stem cell (MSC)-like cells deposited under ECACC accession number 22072103) having reduced FGFR3 expression and/or activity compared to a MSC-like cell deposited under ECACC accession number 22072103. For example, the cell population may comprise derivatives (e.g. of mesenchymal stem cell (MSC)-like cells deposited under ECACC accession number 22072103) wherein the derivative only differs from a MSC-like cell deposited under ECACC accession number 22072103 by having reduced FGFR3 expression and/or activity.

In one example, an extracellular vesicle (EV) population that is obtainable by cell culture of a cell population comprising a plurality of cells, wherein the plurality of cells is made up from (a) at least one mesenchymal stem cell (MSC)-like cell deposited under ECACC accession number 22072103 and (b) at least one derivative of a mesenchymal stem cell (MSC)-like cell deposited under ECACC accession number 22072103 (e.g, wherein the derivative has reduced FGFR3 expression and/or activity compared to a MSC-like cell deposited under ECACC accession number 22072103 and/or wherein the derivative only differs from a MSC-like cell deposited under ECACC accession number 22072103 by having reduced FGFR3 expression and/or activity) is provided.

In a further example, an extracellular vesicle (EV) population that is obtainable by cell culture of a mesenchymal stem cell (MSC)-like cell deposited under ECACC accession number 22072101 is further provided.

An extracellular vesicle (EV) population that is obtainable by cell culture of a cell population comprising a plurality of mesenchymal stem cell (MSC)-like cells deposited under ECACC accession number 22072101 is also provided.

An extracellular vesicle (EV) population that is obtainable by cell culture of a derivative of a mesenchymal stem cell (MSC)-like cell deposited under ECACC accession number 22072101 is also provided.

For example, also provided is an extracellular vesicle (EV) population that is obtainable by cell culture of a cell population comprising a plurality of derivatives of mesenchymal stem cell (MSC)-like cells deposited under ECACC accession number 22072101.

In another example, an extracellular vesicle (EV) population that is obtainable by cell culture of a cell population comprising a plurality of cells, wherein the plurality of cells is made up from (a) at least one mesenchymal stem cell (MSC)-like cell deposited under ECACC accession number 22072101 and (b) at least one derivative of a mesenchymal stem cell (MSC)-like cell deposited under ECACC accession number 22072101, is also provided.

In another example, an extracellular vesicle (EV) population that is obtainable by cell culture of a cell population comprising a plurality of cells, wherein the plurality of cells is made up from (a) at least one mesenchymal stem cell (MSC)-like cell deposited under ECACC accession number 22072103 and (b) at least one mesenchymal stem cell (MSC)-like cell deposited under ECACC accession number 22072101, is also provided.

As used herein, “extracellular vesicle (EV) population” refers to a plurality of extracellular vesicles (EVs) (i.e. 2 or more EVs, for example, 2 or more, 3 or more, 4 or more, 5 or more, 6 or more, 7 or more, 8 or more, 9 or more, 10 or more, 20 or more, 40 or more, 60 or more, 80 or more, 100 or more EVs).

An extracellular vesicle (EV) population as described herein may be obtainable or directly obtained by culturing a cell provided herein or a cell population provided herein in a cell culture medium, thereby conditioning the medium. The conditioned media provided herein (e.g. a cell-free conditioned medium), typically comprise extracellular vesicles (EVs) secreted by the cell or cell population used to condition the medium. An EV population may thus be obtained by isolating a population (i.e. a plurality) of extracellular vesicles from a conditioned medium provided herein (e.g. a cell-free conditioned medium provided herein), thereby obtaining an EV population. The cell culture conditions and parameters provided above in the context of obtaining a conditioned medium therefore apply equally to obtaining a EV population.

An EV population may be isolated from the cell or population of cells used to condition the medium or a conditioned medium already separated from such cells (i.e. a cell-free conditioned medium as provided herein) by one or more appropriate methods known in the art, see for example, the Thery et al protocol (see Théry, Clotilde, Sebastian Amigorena, Graça Raposo, and Aled Clayton. 2006. “Isolation and Characterization of Exosomes from Cell Culture Supernatants and Biological Fluids.” Current Protocols in Cell Biology/Editorial Board, Juan S. Bonifacino . . . [et al.] Chapter 3). Further, suitable methods include differential centrifugation, density-gradient ultracentrifugation, and size exclusion chromatography. Differential centrifugation and density-gradient ultracentrifugation are the most widely applied methods for isolating EVs.

Differential ultracentrifugation may be used to separate different EV subpopulations based on their density. For instance, in some examples, EVs may be isolated by differential centrifugation of conditioned media at increasing centrifugation speed to yield different fractions.

A method for obtaining EVs is provided in the examples section below, however, any suitable alternative methods may also be used. In one example, the EVs may be obtained from serum-free cell conditioned media, wherein the EVs are isolated according to the following main principles from the Thery et al protocol (see Théry, Clotilde, Sebastian Amigorena, Graça Raposo, and Aled Clayton. 2006. “Isolation and Characterization of Exosomes from Cell Culture Supernatants and Biological Fluids.” Current Protocols in Cell Biology/Editorial Board, Juan S. Bonifacino . . . [et al.] Chapter 3).

In some examples, EVs may be obtained via the following method in which cells are seeded in T175 flasks with medium comprising FBS, wherein the EVs within FBS have been depleted, up until reaching 80-90% confluence. In this method, medium is aspirated and cells are washed with PBS, and serum free medium is added. Medium collections are performed and cells counted. EVs are isolated according to the Thery et al protocol (as referenced above) with minor modifications, wherein all steps are performed at 4 °C. Conditioned medium is ultracentrifuged to isolate the EV fraction/s. All EV pellets can be washed thoroughly and resuspended in particle-free PBS, all centrifugations were performed at 4 °C.

EV-depleted FBS can readily be obtained by the skilled person. For example, to obtain EV-depleted FBS, FBS may be centrifuged for 18 hours at 100,000 g, and the supernatant collected.

A detailed protocol for isolating EVs is shown in Example 2 provided below however, as discussed above, any known appropriate method for isolating EVs may alternatively be used. In a particular example, cell-free conditioned media described elsewhere herein may be centrifuged in a Ty45i rotor compatible ultracentrifuge tube (or equivalent) at 100,000 g at 4 degrees Celsius for 90 minutes followed by removal of the supernatant. Resuspension of the pellet may then be performed using cold filtered PBS (e.g. 600 μl per ultracentrifuge tube) followed by pooling of the re-suspended pellets and aliquoting into 1.5 ml micro ultra-centrifuge tubes (or equivalent) with a minimum volume of at least 1 mL per tube. Centrifugation of the micro-ultracentrifuge tubes (or equivalent) may then be performed at 100,000 g for 90 minutes at 4 degrees Celsius followed by removal of the supernatant. Resuspension of each pellet in 50-100 μl of HQ-PBS may then be performed to generate a 100K fraction.

An EV population as described herein may also be referred to as an EV sample that is obtainable by cell culture of a cell or a cell population, e.g. from conditioned media described herein. An EV sample may be a processed sample that is enriched for EVs (i.e. it has a higher concentration of EVs compared to the concentration of EVs in the e.g. conditioned media from which it was generated). In this context, “enriched” or “enrichment” refers to a sample or a process in which the proportion of EVs is increased relative to other components of the sample. Enrichment may be measured by comparing the number of EVs before and after the processing of the sample, where any increase in the relative number of EVs compared to other components of the sample is considered enrichment. Enrichment and/or purity may be measured in terms of concentration compared to the conditioned media (e.g. the unprocessed sample) from which the EV sample has been generated, wherein the concentration of EV's is at least 10%, 15%, 20%, 25%, 30%, 35%, 40%, 45%, 50%, 55%, 60%, 65%, 70%, 75%, 80%, 85%, 90%, 95% or more higher than the concentration of EVs in the conditioned media (e.g. the unprocessed sample). Enrichment and/or purity may be measured in terms of the number EVs such that a sample is enriched about or at least about 2×, 3×, 4×, 5×, 10×, 15×, 20×, 25×, 30×, 35×, 40×, 45×. 50×. 55×, 60×, 65×, 70×, 75×, 80×, 85×, 90×, 95×, 100×, 110×, 120×, 130×, 140×, 150×, 160×, 170×, 180×, 190×, 200×, 210×, 220×, 230×, 240×, 250×, 260×, 270×, 280×, 290×, 300×, 325×, 350×, 375×, 400×, 425×, 450×, 475×, 500×, 525×, 550×, 575×, 600×, 625×, 650×, 675×, 700×, 725×, 750×, 775×, 800×, 825×, 850×, 875×, 900×, 925×, 950×, 975×, 1000×, 1100×, 1200×, 1300×, 1400×, 1500×, 1600×, 1700×, 1800×, 1900×, 2000× (same as-fold)- and all ranges derivable therein—in EVs compared to the conditioned media (e.g. the unprocessed sample) from which the EV sample has been generated. The level of enrichment may be determined using EM and tunable resistive pulse sensing (TRPS).

An EV sample (in other words an EV enriched sample) does not need to be 100% pure extracellular vesicles. Preferably, the EV sample has a minimal amount of contaminating cellular or extracellular content (e.g. cellular or extracellular proteins, lipids, carbohydrates, lipoproteins etc that are not associated with EVs). In other words, the EV sample may be predominantly composed of EVs. In this context, a “minimal amount” may include less than 10% (by concentration) of contaminants, less than 5%, less than 2%, less than 1%, less than 0.5%, less than 0.25%, less than 0.1% etc (by concentration) contaminants. The level of contamination does not need to be 0%. The level of contamination may be determined using EM.

The EV sample may be an isolated sample containing substantially pure EVs. The isolated sample may be isolated from any EV-containing conditioned media. The term “substantially pure” or “substantial purity” when referring to an isolated sample containing substantially pure EVs means the percentage of EVs in the population is significantly higher than that found in a conditioned media (e.g. the unprocessed sample) from which the EV sample has been generated. Typically, the percentage of EVs in an isolated sample containing substantially pure EVs is at least about 50%, preferably at least about 60%, 70%, 75%, and more preferably at least about 80%, 85%, 90%, 95%, 96%, 97%, 98% or 99% of the total sample.

In some examples, the EV population is a 2K EV fraction, a 10K EV fraction or a 100K EV fraction. As is described in the examples section below, and as would be known to a person of skill in the art, a 2K EV fraction refers to the fraction (or portion) of EVs within a secretome or conditioned medium that is generated when the secretome or conditioned medium is centrifuged at 2,000 g (e.g. for 20 minutes). Similarly, a 10K EV fraction refers to the fraction (or portion) of EVs within a secretome or conditioned medium that is generated when the secretome or conditioned medium is centrifuged at 10,000 g (e.g. the fraction (or portion) of EVs within a secretome or conditioned medium that is generated when the secretome or conditioned medium is centrifuged at 10,000 g for 45 minutes, e.g. the fraction (or portion) of EVs within a secretome or conditioned medium that is generated when the secretome or conditioned medium is centrifuged at 10,000 g for 45 minutes twice). In addition, a 100K EV fraction refers to the fraction (or portion) of EVs within a secretome or conditioned medium that is generated when the secretome or conditioned medium is centrifuged at 100,000 g (e.g. the fraction (or portion) of EVs within a secretome or conditioned medium that is generated when the secretome or conditioned medium is centrifuged at 100,000 g for 90 minutes, e.g. the fraction (or portion) of EVs within a secretome or conditioned medium that is generated when the secretome or conditioned medium is centrifuged at 100,000 g for 90 minutes twice). “Fraction” is defined elsewhere herein and also applies to this context. For example, centrifugation may be performed at 4 degrees Celsius.

As described in the examples section below, a 100K EV fraction may also be identified by the presence one or more EV markers selected from the group consisting of: Alix, Flotillin-1, CD81 and CD63. The 100K EV fraction may also be characterised by a lack of the endosomal marker BiP. In other words, the 100K EV fraction may be identified by one or more positive markers selected: from Alix, Flotillin-1, CD81 and CD63, and/or the negative marker BiP. In one example, the 100K EV fraction may be identified by two or more, or three or more positive markers selected: from Alix, Flotillin-1, CD81 and CD63, and/or the negative marker BiP.

The inventors performed full proteomic screening of Y201 conditioned medium and Y201 EVs. Table 2 below provides the top 200 identified proteins in Y201 conditioned medium (i.e. the entire secretome, including EVs). Table 3 below provides the top 200 identified proteins in Y201 EVs. Additionally, the inventors further performed full microRNA screening of Y201 EVs. Table 4 below provides identified miRNAs in Y201 EVs.

In some examples, the cell-free conditioned medium or the secretome, or portion thereof provided herein (e.g. when obtained by culturing of Y201 cells) may comprise at least 10 different proteins selected from Table 2. For example, the cell-free conditioned medium or the secretome, or portion thereof provided herein may comprise at least 10, at least 20, at least 30, at least 40, at least 50, at least 60, at least 70, at least 80, at least 90, at least 100, at least 110, at least 120, at least 130, at least 140, at least 150, at least 160, at least 170, at least 180, at least 190 or 200 different proteins selected from Table 2. For example, the cell-free conditioned medium or the secretome, or portion thereof provided herein may comprise at least 10, at least 20, at least 30, at least 40 or at least 50 different proteins selected from Table 2.

In some examples, the cell-free conditioned medium or the secretome, or portion thereof provided herein (e.g. when obtained by culturing of Y201 cells) may comprise at least the top 10 different proteins selected from Table 2 (wherein the proteins are listed in order of abundance). For example, the cell-free conditioned medium or the secretome, or portion thereof provided herein may comprise at least the top 10, at least the top 20, at least the top 30, at least the top 40, or at least the top 50 different proteins selected from Table 2.

In some examples, the cell-free conditioned medium or the secretome, or portion thereof provided herein (e.g. when obtained by culturing of Y201 cells) may comprise 10 different proteins selected from Table 2. For example, the cell-free conditioned medium or the secretome, or portion thereof provided herein may comprise 10, 20, 30, 40, 50, 60, 70, 80, 90, 100, 110, 120, 130, 140, 150, 160, 170, 180, 190 or 200 different proteins selected from Table 2. For example, the cell-free conditioned medium or the secretome, or portion thereof provided herein may comprise 10, 20, 30, 40 or 50 different proteins selected from Table 2.

In some examples, the cell-free conditioned medium, the secretome, or portion thereof or the EV population provided herein (e.g. when obtained by culturing of Y201 cells) may comprise at least 10 different proteins selected from Table 3. For example, the cell-free conditioned medium, the secretome, or portion thereof or the EV population provided herein may comprise at least 10, at least 20, at least 30, at least 40, at least 50, at least 60, at least 70, at least 80, at least 90, at least 100, at least 110, at least 120, at least 130, at least 140, at least 150, at least 160, at least 170, at least 180, at least 190 or 200 different proteins selected from Table 3. For example, the cell-free conditioned medium, the secretome, portion thereof or the EV population provided herein may comprise at least 10, at least 20, at least 30, at least 40 or at least 50 different proteins selected from Table 3.

In some examples, the cell-free conditioned medium, the secretome, or portion thereof or the EV population provided herein (e.g. when obtained by culturing of Y201 cells) may comprise at least the top 10 different proteins selected from Table 3 (wherein the proteins are listed in order of abundance). For example, the cell-free conditioned medium, the secretome, or portion thereof or the EV population provided herein may comprise at least the top 10, at least the top 20, at least the top 30, at least the top 40 or at least the top 50 different proteins selected from Table 3.

In some examples, the cell-free conditioned medium, the secretome, or portion thereof or the EV population provided herein (e.g. when obtained by culturing of Y201 cells) may comprise 10 different proteins selected from Table 3. For example, the cell-free conditioned medium, the secretome, or portion thereof or the EV population provided herein may comprise 10, 20, 30, 40, 50, 60, 70, 80, 90, 100, 110, 120, 130, 140, 150, 160, 170, 180, 190 or 200 different proteins selected from Table 3. For example, the cell-free conditioned medium, the secretome, or portion thereof or the EV population provided herein may comprise 10, 20, 30, 40 or 50 different proteins selected from Table 3.

In some examples, the cell-free conditioned medium, the secretome, or portion thereof or the EV population provided herein (e.g. when obtained by culturing of Y201 cells) may comprise at least 10 different miRNAs selected from Table 4. For example, the cell-free conditioned medium, the secretome, or portion thereof or the EV population provided herein may comprise at least 10, at least 20, at least 30, at least 40, at least 50, at least 60, at least 70, at least 80, at least 90, at least 100, at least 110, at least 120, at least 130, at least 140, at least 150, at least 160, at least 170, at least 180, at least 190 or 200 different miRNAs selected from Table 4. For example, the cell-free conditioned medium, the secretome, portion thereof or the EV population provided herein may comprise at least 10, at least 20, at least 30, at least 40 or at least 50 different miRNAs selected from Table 4.

In some examples, the cell-free conditioned medium, the secretome, or portion thereof or the EV population provided herein (e.g. when obtained by culturing of Y201 cells) may comprise at least the top 10 different miRNAs selected from Table 4 (wherein the miRNAs are listed in order of abundance). For example, the cell-free conditioned medium, the secretome, or portion thereof or the EV population provided herein may comprise at least the top 10, at least the top 20, at least the top 30, at least the top 40 or at least the top 50 different miRNAs selected from Table 4.

In some examples, the cell-free conditioned medium, the secretome, or portion thereof or the EV population provided herein (e.g. when obtained by culturing of Y201 cells) may comprise 10 different miRNAs selected from Table 4. For example, the cell-free conditioned medium, the secretome, or portion thereof or the EV population provided herein may comprise 10, 20, 30, 40, 50, 60, 70, 80, 90, 100, 110, 120, 130, 140, 150, 160, 170, 180, 190 or 200 different miRNAs selected from Table 4. For example, the cell-free conditioned medium, the secretome, or portion thereof provided herein or the EV population provided herein may comprise 10, 20, 30, 40 or 50 different miRNAs selected from Table 4.

Methods for detecting the presence of miRNA and/or protein in a sample are well known, and include ELISA, mass spectrometry, such as liquid chromatography-mass spectrometry (LC-MS), Northern blotting, in situ hybridization, reverse transcription qPCR, microarray, and next-generation sequencing etc.

In some examples, miRNAs may be detected using the NanoString nCounter Human v3 miRNA expression assay codeset (NanoString) (e.g. as set out in the examples section below). In this particular example, MiRNA counts may be normalized using spike-ins from the NanoString procedure before miRNAs with >20 counts in ≥1 sample are filtered for analysis. LC-MS/MS may be used to detect EV proteins, peptide identifications may be filtered through the Percolator algorithm to achieve a global 1% false discovery rate (FDR). Identifications may be imported back into Progenesis QI and mapped onto MS1 peak areas. Peak areas may be normalized to total ion intensity for all identified peptides. Relative protein quantification may be performed using relative peak areas of non-conflicting peptides. Proteins may be accepted for analysis if they are detected with ≥2 peptides and ≥1 unique-peptide in at least one sample.

A composition comprising at least 10 different proteins selected from Table 2 is provided.

In one example, the composition may comprise at least 10, at least 20, at least 30, at least 40, at least 50, at least 60, at least 70, at least 80, at least 90, at least 100, at least 110, at least 120, at least 130, at least 140, at least 150, at least 160, at least 170, at least 180, at least 190 or 200 different proteins selected from Table 2. For example, the composition may comprise at least 10, at least 20, at least 30, at least 40 or at least 50 different proteins selected from Table 2. In some examples, the composition may comprise 10 different proteins selected from Table 2. For example, the composition may comprise 10, 20, 30, 40, 50, 60, 70, 80, 90, 100, 110, 120, 130, 140, 150, 160, 170, 180, 190 or 200 different proteins selected from Table 2. For example, the composition may comprise 10, 20, 30, 40 or 50 different proteins selected from Table 2.

A composition comprising at least 10 different proteins selected from Table 3 is further provided.

In one example, the composition may comprise at least 10, at least 20, at least 30, at least 40, at least 50, at least 60, at least 70, at least 80, at least 90, at least 100, at least 110, at least 120, at least 130, at least 140, at least 150, at least 160, at least 170, at least 180, at least 190 or 200 different proteins selected from Table 3. For example, the composition may comprise at least 10, at least 20, at least 30, at least 40 or at least 50 different proteins selected from Table 3. In some examples, the composition may comprise 10 different proteins selected from Table 3. For example, the composition may comprise 10, 20, 30, 40, 50, 60, 70, 80, 90, 100, 110, 120, 130, 140, 150, 160, 170, 180, 190 or 200 different proteins selected from Table 3. For example, the composition may comprise 10, 20, 30, 40 or 50 different proteins selected from Table 3.

A composition comprising at least 10 different miRNAs selected from Table 4 is also provided.

In one example, the composition may comprise at least 10, at least 20, at least 30, at least 40, at least 50, at least 60, at least 70, at least 80, at least 90, at least 100, at least 110, at least 120, at least 130, at least 140, at least 150, at least 160, at least 170, at least 180, at least 190 or 200 different miRNAs selected from Table 4. For example, the composition may comprise at least 10, at least 20, at least 30, at least 40 or at least 50 different miRNAs selected from Table 4. In some examples, the composition may comprise 10 different miRNAs selected from Table 4. For example, the composition may comprise 10, 20, 30, 40, 50, 60, 70, 80, 90, 100, 110, 120, 130, 140, 150, 160, 170, 180, 190 or 200 different miRNAs selected from Table 4. For example, the composition may comprise 10, 20, 30, 40 or 50 different miRNAs selected from Table 4.

In some examples, the composition is a cell-free composition. In other words, the composition essentially does not contain cells (e.g. mesenchymal stem cell (MSC)-like cells deposited under ECACC accession number 22072103, derivatives thereof, mesenchymal stem cell (MSC)-like cells deposited under ECACC accession number 22072101 and/or derivatives thereof). “Cell-free” is defined in more detail elsewhere herein.

The inventors have demonstrated that the cells, cell populations, cell-free conditioned media, secretomes, and/or EV populations described herein have therapeutic utility.

Thus, a pharmaceutical composition is provided comprising a MSC-like cell, a cell population, a DNA preparation, a cell-free conditioned medium, a secretome, or a portion thereof, an extracellular vesicle (EV) population, or a composition described herein, wherein the pharmaceutical composition further comprises a pharmaceutically acceptable adjuvant, excipient, diluent and/or carrier.

A MSC-like cell, a cell population, a DNA preparation, a cell-free conditioned medium, a secretome, or a portion thereof, an extracellular vesicle (EV) population, or a composition, described herein may therefore be provided as part of a pharmaceutical composition.

Pharmaceutical compositions may routinely contain pharmaceutically acceptable concentrations of salt, buffering agents, preservatives, compatible carriers, supplementary immune potentiating agents such as adjuvants and cytokines and optionally other therapeutic agents or compounds.

As used herein, “pharmaceutically acceptable” refers to a material that is not biologically or otherwise undesirable, i.e., the material may be administered to an individual along with the selected MSC-like cell, cell population, DNA preparation, cell-free conditioned medium, secretome, or portion thereof, extracellular vesicle (EV) population, or composition without causing any undesirable biological effects or interacting in a deleterious manner with any of the other components of the pharmaceutical composition in which it is contained.

Excipients are natural or synthetic substances formulated alongside an active ingredient (e.g. a MSC-like cell, a cell population, a DNA preparation, a cell-free conditioned medium, a secretome, or a portion thereof, an extracellular vesicle (EV) population, or a composition as provided herein), included for the purpose of bulking-up the formulation or to confer a therapeutic enhancement on the active ingredient in the final dosage form, such as facilitating drug absorption or solubility. Excipients can also be useful in the manufacturing process, to aid in the handling of the active substance concerned such as by facilitating powder flowability or non-stick properties, in addition to aiding in vitro stability such as prevention of denaturation over the expected shelf life. Pharmaceutically acceptable excipients are well known in the art. A suitable excipient is therefore easily identifiable by one of ordinary skill in the art. By way of example, suitable pharmaceutically acceptable excipients include water, saline, aqueous dextrose, glycerol, ethanol, and the like.

Adjuvants are pharmacological and/or immunological agents that modify the effect of other agents in a formulation. Pharmaceutically acceptable adjuvants are well known in the art. A suitable adjuvant is therefore easily identifiable by one of ordinary skill in the art.

Diluents are diluting agents. Pharmaceutically acceptable diluents are well known in the art. A suitable diluent is therefore easily identifiable by one of ordinary skill in the art.

Carriers are non-toxic to recipients at the dosages and concentrations employed and are compatible with other ingredients of the formulation. The term “carrier” denotes an organic or inorganic ingredient, natural or synthetic, with which the active ingredient is combined to facilitate the application. Pharmaceutically acceptable carriers are well known in the art. A suitable carrier is therefore easily identifiable by one of ordinary skill in the art.

The pharmaceutical compositions described herein may be administered to a subject as a monotherapy or as part of a combination therapy. For example, combinations of a MSC-like cell, a cell population, a DNA preparation, a cell-free conditioned medium, a secretome, or a portion thereof, an extracellular vesicle (EV) population, or a composition as provided herein with doxorubicin may be particularly useful, e.g. for the treatment of certain types of cancerous tumours. Administration of the combination may be in any order (preferably, the pharmaceutical composition is administered at the same time or after the doxorubicin, alternatively, the pharmaceutical composition is administered at the same time or before the doxorubicin).

The pharmaceutical compositions described herein may advantageously be used as a medicament. The pharmaceutical compositions may be used in promoting tissue repair, wound healing and/or tissue regeneration, or in treating or preventing inflammation.

The pharmaceutical compositions for use as a medicament (e.g. in promoting tissue repair or in treating or preventing inflammation) may comprise a MSC-like cell, a cell population, a DNA preparation, a cell-free conditioned medium, a secretome, or a portion thereof, an extracellular vesicle (EV) population, or a composition as described herein along with a pharmaceutically acceptable excipient, adjuvant, diluent and/or carrier.

In some examples, the pharmaceutical composition provided herein may be for use in promoting tissue repair. As would be clear to the skilled person a method of promoting tissue repair and/or regeneration described herein results in improved tissue repair in the subject.

“Tissue repair”, as used herein, refers to anatomical and/or functional restoration of the condition of the tissue prior to damage (e.g. prior to injury). In other words, tissue repair involves restoration of the anatomy and/or function of a damaged tissue such that the repaired tissue more closely resembles the anatomy and/or function of the tissue prior to damage. “Tissue repair” as used herein may refer to partial or complete restoration of the anatomy and/or function of a damaged tissue (for example, in comparison to the anatomy and/or function of the tissue prior to damage). A non-damaged (i.e. a healthy) tissue of the same type as the damaged tissue may be used for anatomical and/or functional comparison to determine the condition of the damage tissue. Tissue repair may include restoration of physical continuity between portions of tissue that were separated (e.g. by an injury). Preferably such restoration of physical continuity includes reapposition or reconnection of the portions of tissue without appreciable separation by tissue of a type that was not present prior to damage (e.g. prior to injury), such as scar tissue. Repair may thus include filling of a tissue defect, e.g., by reapposition of portions of tissue separated by the defect and/or by growth of new tissue of the type that was subject to damage or degradation, rather than by development of scar tissue. Repair may include growth or development of new tissue. Thus tissue repair may include tissue regeneration, however, in some examples, repair can occur without evidence of new tissue growth. A skilled person would readily be able to identify tissue repair using routine methods known in the art. For example, the skilled person may visually compare a damaged tissue with a healthy tissue of the same type at different time points after treatment with a pharmaceutical compositions provided herein.

In some examples, the pharmaceutical composition provided herein may be for use in promoting tissue regeneration. As would be clear to the skilled person a method of promoting tissue regeneration described herein results in improved tissue regeneration in the subject.

“Tissue regeneration”, as used herein, includes any aspect of anatomical and/or functional restoration of the condition of the tissue prior to damage (e.g. damage via an injury or a degenerative or degradative process) which involves production of new tissue (by which is meant either cells or portions of cells). “Tissue regeneration” as used herein may refer to partial or complete regeneration of the anatomy and/or function of a damaged tissue. Production of new tissue may include growth of existing cells. For example, in the case of chondrocytes, regeneration may comprise increased cell growth, increased rate and extent of chondrogenic differentiation, increased cartilage tissue formation. For example, in the case of osteoblasts, regeneration may comprise increased cell growth, increased rate and extent of osteogenic differentiation, increased bone tissue formation. For example, in the case of adipocytes, regeneration may comprise increased cell growth, increased rate and extent of adipogenic differentiation, increased adipose tissue formation. The new tissue may replace tissue that was previously present. Production of new tissue may include division of existing cells. In some examples, regeneration results in the re-establishment of the original tissue structure and function.

Tissue damage typically impairs the function and/or anatomy of a tissue. A person of skill in the art would readily be able to identify a damaged (e.g. injured) tissue using routine methods known in the art (e.g. visually, for example assisted by microscopy). Tissue damage may occur due to injury (e.g. trauma), a disease, a disorder or a condition, degeneration and/or a degradative process.

As is known to a person of skill in the art, a tissue is a group of cells that have similar structure and that function together as a unit. As used herein, “tissue” includes any external or internal bodily tissue. Examples of tissues include, but are not limited to, brain, skin, liver, pancreas, stomach, kidney, gastrointestinal tract, esophageal tract, heart, muscle, connective tissue, cartilage, nerve, fat, or bone marrow tissue. Tissues particularly relevant in the context of the present invention include cartilage, bone and fat tissue.

Accordingly, in some examples, the pharmaceutical composition provided herein is for use in treating or preventing a disease or condition associated with cartilage, bone and/or adipose tissue damage.

Accordingly, in some examples, the pharmaceutical composition provided herein is for use in treating or preventing a disease or condition associated with cartilage damage.

Cartilage is a connective tissue found in many parts of the body. Cartilage has several functions, including holding bones together and supporting other tissues. A person of skill in the art may readily identify cartilage damage in a subject, for example, using MRI or arthroscopy, and thus a disease or condition associated with cartilage damage. Diseases or conditions associated with cartilage damage are well known to a person of skill in the art. Non-limiting examples of diseases or conditions associated with cartilage damage include arthritis. Arthritis is defined as an acute or chronic joint inflammation in the joint. Arthritis may attribute to a wide variety of symptoms that include pain, stiffness, decreased range of motion, and joint deformities. There are several different types of arthritis. Non-limiting examples of different types of arthritis include juvenile idiopathic arthritis, rheumatoid arthritis, osteoarthritis, spondyloarthritis and psoriatic arthritis.

In some examples, the pharmaceutical composition provided herein is for use in treating or preventing arthritis.

Pharmaceutical compositions provided herein that are particularly relevant for treating or preventing arthritis are pharmaceutical compositions comprising an extracellular vesicle population described herein, in particular an extracellular vesicle (EV) population that is obtainable by cell culture of a Y201 cell or population thereof.

In some examples, the pharmaceutical composition provided herein is for use in treating or preventing inflammatory arthritis.

Inflammatory arthritis is an umbrella term used for a group of arthritic conditions which cause pain, stiffness and damage to joints, examples include but are not limited to rheumatoid arthritis, psoriatic arthritis, ankylosing spondylitis, and juvenile idiopathic arthritis subtypes. Accordingly, in some examples, the arthritis (e.g. the inflammatory arthritis) is selected from the group consisting of: rheumatoid arthritis, psoriatic arthritis, ankylosing spondylitis, and juvenile idiopathic arthritis. Key features of inflammatory arthritis include signs of inflammation, such as redness, swelling, and warmth around the joint. Clinicians may look for particular markers in the blood, such as rheumatoid factors, C-reactive protein, or particular antibodies, like anti-CCP, in order to identify inflammatory arthritis. A person of skill in the art would readily be able to identify inflammatory arthritis.

In some examples, the arthritis is selected from the group consisting of: rheumatoid arthritis, psoriatic arthritis, ankylosing spondylitis, juvenile idiopathic arthritis, osteoarthritis, and spondyloarthritis.

In some examples, the arthritis is selected from the group consisting of: juvenile idiopathic arthritis, rheumatoid arthritis, gout, osteoarthritis, spondyloarthritis, ankylosing spondylitis, post-traumatic osteoarthritis, reactive arthritis and psoriatic arthritis.

In some examples, the arthritis is selected from the group consisting of: juvenile idiopathic arthritis, rheumatoid arthritis, osteoarthritis, spondyloarthritis and psoriatic arthritis. In some examples, the arthritis is juvenile idiopathic arthritis or psoriatic arthritis.

In some examples, the pharmaceutical composition provided herein may be for use in wound healing.

A wound occurs when the structure and/or integrity of one or several tissues is compromised, for example when skin breaks, muscles tear, a bone is fractured or tissue is burned. Accordingly, as would be clear to the skilled person, a wound is an example of tissue damage. The skilled person would thus readily understand that wound healing is an example of tissue repair (and, that wound healing may involve tissue regeneration). A wound may be caused by an accident, trauma or a medical procedure, by an infectious disease or an underlying disease condition for example, wounds may also have other causes. Depending on the location, extent and severity of a wound it can be classified as closed or open. Wound healing occurs in three distinct phases. The inflammatory phase is characterized by inflammation at the site of the trauma. This phase is critical for healing and involves extensive cell migration. The second phase of wound healing is the proliferative phase, which is marked by epithelialization, angiogenesis, granulation tissue formation and collagen deposition. Angiogenesis, which involves new capillary formation, is used to deliver nutrients and maintain granulation. Without formation of new capillaries into the wound, required nutrients fail to reach the wound resulting in a chronically unhealed wound. The third and final stage of wound healing is the maturational phase wherein fibroblasts differentiate into collagen. The disposition of the connective tissue matrix and collagen undergoes a contraction, resulting in scar tissue. Although scar formation is critical to wound healing, excessive scar formation can have additional cosmetic and/or pathologic consequences, such as keloids and/or hypertrophic scars.

Wound healing may be determined using routine methods known in the art, for example wound healing may be conveniently measured by a reduction in wound area over a defined period of time (e.g. over 5 days, over 10 days, over 15 days or over 20 days). In other words, wound healing may be measured by comparing wound area at different time points after a wound is obtained, wherein a reduction in wound area over time is indicative of wound healing.

In some examples, the wound may be an acute or a chronic wound. Acute wounds are those wounds that heal promptly, within 30 days (or 60 days in diabetics). Non-limiting examples of acute wounds that can be treated with the present invention include abrasions, avulsions, contusions, crush wounds, cuts, lacerations, projectile wounds and puncture wounds. Chronic wounds include, but are not limited to, diabetic skin sores, pressure sores, surgical wounds, spinal injury wounds, burns, chemical-induced wounds and wounds due to blood vessel disorders.

In some examples, the pharmaceutical composition provided herein may be for use in treating or preventing inflammation.

The method of treating or preventing inflammation described herein typically results a reduction of inflammation (e.g. a reduction in localised inflammation). A reduction in inflammation may be determined using routine methods known in the art, for example, a reduction in inflammation may be conveniently measured by a reduction in symptoms of inflammation and/or a reduction in the area of inflammation over a defined period of time (e.g. over 5 days, over 10 days, over 15 days or over 20 days). For example, a reduction in inflammation may be measured by comparing inflammation at different time points after inflammation occurs. In some examples, the level of inflammation (e.g. a reduction in the level of inflammation) may be determined by measuring biomarkers of inflammation known to a person of skill in the art (e.g. C-reactive protein, IL-6 and/or TNF-α) in a subject using routine methods.

Inflammation typically refers to a protective response elicited by damage of a tissue (e.g. injury or destruction of a tissue). Inflammation typically serves to destroy, dilute, or wall off (sequester) both the injurious agent and the damaged tissue and is notably associated with influx of leukocytes and/or neutrophil chemotaxis. Inflammation is typically characterised by classic signs and symptoms, including edema, erythema (redness), warmness, pain, and loss of function (stiffness and immobility) as is known to a person of skill in the art. In healthy individuals inflammation is self-limiting, and resolution is controlled by the release of anti-inflammatory mediators and cytokines, such as interleukin-10 (IL-10), produced by cells called ‘suppressive’ or ‘regulatory’ which are produced as part of a negative feedback loop. Indeed, in the normal process of inflammation in the body, an initial pro-inflammatory response is followed by a pro-resolution response which turns the inflammation off after the insult has been resolved, leading to the reduction of pro-inflammatory cytokines such as TNFα and IL-12, coupled with increased levels of anti-inflammatory cytokines such as IL-10 and TGF-β, resulting in the generation of a so-called tolerogenic environment.

Inflammation is part of many disease states. For example, inflammation may result from infection with pathogenic organisms and/or viruses, or from non-infectious means, such as trauma, injury, toxic chemicals, overuse, detection of a foreign antigen, and/or an autoimmune response. Accordingly, as would be clear to the skilled person, administration of the compositions described herein to a subject may be beneficial in a number of different contexts. For example, administration of such compositions to a subject may be beneficial in any context where inflammation control may be beneficial (e.g. in the treatment of persistent infection, where continued inflammation caused by infection can result in tissue damage). Non-limiting examples of contexts in which administration of the pharmaceutical composition provided herein to a subject may be beneficial include allergies, infection (e.g. persistent/chronic infection) pulmonary diseases, diabetes, neurological disease, cardiovascular disease, bowel disease, trauma, graft versus host disease, periodontal disease, cancers. Thus, in some examples, the pharmaceutical composition provided herein may be for use in treating or preventing allergies, infection (e.g. persistent/chronic infection) pulmonary diseases, diabetes, neurological disease, cardiovascular disease, bowel disease, trauma, graft versus host disease, periodontal disease, and/or cancers. As described elsewhere herein, the pharmaceutical compositions provided herein may be used alongside (e.g. in combination) with other treatments or treatment regimens. A person of skill in the art would readily be able to identify inflammation using routine methods known in the art and accordingly, diseases, conditions and/or disorders that may benefit from treatment with the pharmaceutical compositions provided herein.

Inflammation may be acute or chronic. Accordingly, the pharmaceutical compositions provided here may be for use in treating or preventing acute and/or chronic inflammation. For example, the pharmaceutical compositions provided herein may be for use in treating or preventing acute and/or chronic inflammation resulting from a wound (this may promote wound healing and/or prevent tissue damage in the case of chronic inflammation).

In some examples, the pharmaceutical composition provided herein may be for use in treating and/or preventing infection.

Inflammation is implicated in many autoimmune disease and conditions (including arthritis, discussed above).

In some examples, the pharmaceutical composition provided herein may be for use in treating or preventing an autoimmune disease or condition. As would be clear to the skilled person, administration of such compositions to a subject may be beneficial in a number of different contexts. For example, GvHD, multiple sclerosis, psoriasis, eczema, autoimmune disorders of the GI tract (e.g. Crohn's), lupus (systemic lupus erythematosus), fibromyalgia, (inflammatory) arthritis. Accordingly, in some examples, the pharmaceutical composition provided herein may be for use in treating or preventing GvHD, multiple sclerosis, psoriasis, eczema, autoimmune disorders of the GI tract (e.g. Crohn's), lupus (systemic lupus erythematosus), (inflammatory) arthritis and/or fibromyalgia. In a particular example, the pharmaceutical composition provided herein may be for use in treating and/or preventing an autoimmune disease or condition, wherein the autoimmune disease or condition is selected from the group consisting of: GvHD, psoriasis, systemic lupus erythematosus and arthritis, optionally wherein the arthritis is selected from the group consisting of: juvenile idiopathic arthritis, rheumatoid arthritis, osteoarthritis, spondyloarthritis and psoriatic arthritis. Notably, EVs have previously been administered systemically to a therapy-refractive GvHD patient, which improved the condition (Kordelas, L., Rebmann, V., Ludwig, AK. et al. MSC-derived exosomes: a novel tool to treat therapy-refractory graft-versus-host disease. Leukemia 28, 970-973 (2014)).

As used herein, the terms “treat”, “treating” and “treatment” are taken to include an intervention performed with the intention of preventing the development or altering the pathology of a condition, disorder or symptom (e.g. a condition, disorder or symptom described herein). Accordingly, “treatment” refers to both therapeutic treatment and prophylactic or preventative measures, wherein the object is to prevent or slow down (lessen) the targeted condition, disorder or symptom. For example, treatment may refer to amelioration of a condition, disorder or symptom (e.g. arresting a condition, disorder or symptom, or reducing the manifestation, extent or severity of at least one of the clinical symptom thereof). For instance, ‘treating’ or ‘treatment’ may refer to ameliorating at least one physical parameter, which may not be discernible by the subject. In some examples, ‘treating’ or ‘treatment’ refers to modulating a disorder, condition or symptom, either physically, (e.g. stabilization of a discernible symptom), physiologically, (e.g. stabilization of a physical parameter), or both.

The terms “preventing”, “prevent” or “prevention”, as used herein, refer to a reduction in risk of acquiring or developing a condition, disorder or symptom (e.g. a condition, disorder or symptom described herein) (e.g. causing at least one of the symptoms of the condition or disorder not to develop in a subject that may be exposed to a disease-causing agent, or predisposed to the disease or condition in advance of onset).

As used here in the term “subject” refers to an individual, e.g., a human, having or at risk of having a specified condition, disorder or symptom (e.g. as described herein). The subject may be a patient i.e. a subject in need of treatment in accordance with the invention. The subject may have received treatment for the condition, disorder or symptom. Alternatively, the subject has not been treated prior to treatment in accordance with the present invention. Preferably, the subject is a human subject.

The pharmaceutical compositions described herein can be administered to the subject by any conventional route, including injection or by gradual infusion over time. The administration may, for example, be by infusion or by intramuscular, intravascular, intracavity, intracerebral, intralesional, rectal, subcutaneous, intradermal, epidural, intrathecal, percutaneous administration. For example, the administration may be intra-articular.

The pharmaceutical compositions described herein may be in any form suitable for the above modes of administration. For example, compositions comprising cells may in any form suitable for infusion. As further examples, suitable forms for parenteral injection (including, subcutaneous, intramuscular, intravascular or infusion) include a sterile solution, suspension or emulsion; suitable forms for topical administration include an ointment or cream; and suitable forms for rectal administration include a suppository. Alternatively, the route of administration may be by direct injection into the target area, or by regional delivery or by local delivery. The identification of suitable dosages of the pharmaceutical compositions of the invention is well within the routine capabilities of a person of skill in the art.

Preferably, the pharmaceutical composition comprises or consists of an amount of active ingredient (e.g. a MSC-like cell, a cell population, a DNA preparation, a cell-free conditioned medium, a secretome, or a portion thereof, an extracellular vesicle (EV) population, or a composition described herein) that constitutes a pharmaceutical dosage unit. A pharmaceutical dosage unit is defined herein as the amount of active ingredients (i.e. the total amount of a MSC-like cell, a cell population, a DNA preparation, a cell-free conditioned medium, a secretome, or a portion thereof, an extracellular vesicle (EV) population, or a composition described herein in a vaccine for example) that is applied to a subject at a given time point. It is to be understood herein that the separate volumes of a pharmaceutical dosage may differ in composition, i.e. may comprise different kinds or composition of active ingredients and/or adjuvants.

The pharmaceutical compositions described herein are for administration in an effective amount. An “effective amount” is an amount that alone, or together with further doses, produces the desired (therapeutic or non-therapeutic) response. The effective amount to be used will depend, for example, upon the therapeutic (or non-therapeutic) objectives, the route of administration, and the condition of the patient/subject. For example, the suitable dosage of the composition of the invention for a given patient/subject will be determined by the attending physician (or person administering the pharmaceutical composition), taking into consideration various factors known to modify the action of the composition of the invention for example severity and type of haematological malignancy, body weight, sex, diet, time and route of administration, other medications and other relevant clinical factors. The dosages and schedules may be varied according to the particular condition, disorder or symptom the overall condition of the patient/subject. Effective dosages may be determined by either in vitro or in vivo methods.

The pharmaceutical compositions of the present invention may advantageously presented in unit dosage form.

Use of an extracellular vesicle (EV) population provided herein for delivery of a cargo to a cell is provided herein.

Suitably, EVs within the EV population may comprise a cargo. The cargo may be any entity such as, without limitation, a chemical compound, a combination of compounds, a supramolecular complex of a synthetic or natural origin, a genetic material, a portion thereof, or a derivative thereof, that is capable of having a useful property or exerting a useful activity. The cargo may be hydrophilic or hydrophobic.

Suitably, the cargo may be an active pharmaceutical ingredient, optionally wherein the active pharmaceutical ingredient is selected from the group consisting of: a small molecule, peptide, protein, inorganic nanoparticle, oligonucleotide, or any combination thereof. Where compatible, terms “active pharmaceutical ingredient” and “drug” are used interchangeably herein.

Suitably, the cargo may be an imaging agent, optionally wherein the imaging agent is selected from: MRI contrast agents (e.g. Gd), PET/SPECT radioactive imaging agents (e.g. 111 In, 64Cu), paramagnetic nanoparticles (e.g. iron oxide), fluorescent probes, bioluminescent probes, quantum dots, gold nanoparticles, optical coherence tomography agents (e.g. gold nanorods, fluorescent proteins, fluorescent/radioactive latex beads/polymers, photoacoustic imaging agents (eg carbon nanotubes), Raman spectroscopy agents (e.g. AuNPs), nanobubbles, or any combination thereof.

The skilled person will appreciate that any active pharmaceutical ingredient or any imaging agent may be used in the context of the invention. Non-limiting examples are provided below.

For the avoidance of doubt, an EV may contain a plurality of cargoes e.g. two or more active pharmaceutical ingredients, two or more imaging agents, or a mixture of active pharmaceutical ingredients and imaging agents.

A cargo (e.g. an imaging agent) may be bound to (or adsorbed onto, or tethered to) the outer surface of the EV. This would be advantageous if, for example, they are being used in diagnostic methods, or for targeted delivery using antibodies or aptamers. Alternatively, a cargo may be incorporated into the EV. For example, the cargo may be encapsulated within and/or covalently bound to said EVs.

In some examples, the cargo may be an siRNA. This may be advantageous in the context of gene therapies, specifically in the delivery of gene therapies, which has historically been a major obstacle to successful gene therapy.

The cargo may be encapsulated by at least one lipid bilayer of the EV such that it is located within the aqueous core. It is to be expected that while a substantial proportion of that active pharmaceutical ingredient or imaging agent will be encapsulated within the EV, a proportion may not be encapsulated therein. By reference to substances being contained “within” EVs, it is intended to include substances may be wholly encapsulated within the EV structure (e.g. within the lipid bilayer wall(s), or within a region that is enclosed within that lipid bilayer wall(s)). Alternatively, the substance may be covalently bound to one or more constituents of the EV particle, e.g. the lipid bilayer structure in the case of EVs.

The EVs may be prepared to contain the desired cargo. The process of incorporation of a desired cargo into an EV is often referred to as “loading”. The EV-incorporated cargo may be completely or partially located in the interior space of the EV, within the bilayer membrane of the EV, or associated with the exterior surface of the EV membrane. The incorporation of cargo into EVs is also referred to as encapsulation or entrapment, and these three terms are used herein interchangeably with the same meaning.

The intent of the EV encapsulation of cargo is often to protect the cargo from the destructive environment while providing an opportunity for the encapsulated cargo to exert its activity mostly at the site or in the environment where such activity is advantageous but less so in other sites where such activity may be useless or undesirable.

This phenomenon is referred to as delivery. For example, a drug substance within the EV can be protected from the destruction by enzymes in the body, but become released from the EV and provide treatment at the site of disease.

EVs and their cargo may be delivered to recipient cells via a range of mechanisms, for a review, see Gurung et al, 2021 The exosome journey: from biogenesis to uptake and intracellular signalling. Cell Commun Signal 19, 47 (2021). In some examples, EVs are taken up by the cell, where contents are released. EVs can interact with the plasma membrane of recipient cells, for example through integrins and adhesion molecules, and be internalised for example via clathrin-mediated endocytosis, phagocytosis, lipid raft-mediated endocytosis, caveolin-mediated endocytosis and/or pinocytosis to deliver their cargo.

In some examples, EVs and their cargo may be delivered to recipient cells via integrin-mediated endocytosis (e.g. RGD-integrin-mediated endocytosis). Testing whether an EV is taken up by integrin-mediated endocytosis would be routine for a person of skill in the art and suitable tests are described in the examples below.

The recipient cell may be any suitable cell. As would be clear to the skilled person, the recipient cell may dependent on the cargo of the EV population.

In some examples, the recipient cell may be selected from the group consisting of: an MSC cell, an MSC-like cell, an osteosarcoma cell, a macrophage, a T cell, a B cell, an NK cell, a neutrophil, a monocyte, a mast cell, and an eosinophil.

In some examples, the recipient cell may be selected from the group consisting of: an MSC cell, an MSC-like cell and an osteosarcoma cell. In some examples, the recipient cell may be an MSC cell or an MSC-like cell.

The term “in vitro” generally denotes outside, or external to, animal or human body. The term “ex vivo” typically refers to tissues or cells removed from an animal or human body and maintained or propagated outside the body, e.g., in a culture vessel. The term “in vitro” as used herein should be understood to include “ex vivo”. The term “in vivo” generally denotes inside, on, or internal to, animal or human body.

Unless defined otherwise herein, all technical and scientific terms used herein have the same meaning as commonly understood by one of ordinary skill in the art to which this invention pertains. For example, Singleton and Sainsbury, Dictionary of Microbiology and Molecular Biology, 2d Ed., John Wiley and Sons, NY (1994); and Hale and Marham, The Harper Collins Dictionary of Biology, Harper Perennial, NY (1991) provide those of skill in the art with a general dictionary of many of the terms used in the invention. Although any methods and materials similar or equivalent to those described herein find use in the practice of the present invention, the preferred methods and materials are described herein. Accordingly, the terms defined immediately below are more fully described by reference to the Specification as a whole. Also, as used herein, the singular terms “a”, “an,” and “the” include the plural reference unless the context clearly indicates otherwise. Unless otherwise indicated, nucleic acids are written left to right in 5′ to 3′ orientation; amino acid sequences are written left to right in amino to carboxy orientation, respectively. It is to be understood that this invention is not limited to the particular methodology, protocols, and reagents described, as these may vary, depending upon the context they are used by those of skill in the art.

Aspects of the invention are demonstrated by the following non-limiting examples.

### Example 1

As discussed above, to address the challenges associated with the use of MSCs in therapy, the inventors initiated a programme of immortalisation and cloning to deliver a panel of MSC lines representing different bone marrow stromal subtypes, including stem cells of varying potency. From numerous initial clones, 8 were selected based on strong in vitro growth performance for in-depth characterisation. Advantageously, the inventors identified one line in particular, termed Y201, which showed typical MSC characteristics, with potent tissue-forming and anti-inflammatory properties in vitro and in vivo.

The inventors have shown that Y201 MSCs are highly reproducible and have thoroughly characterised these cells (e.g. their growth, transcriptomics, secretomics, surfaceomics, and marker expression). The inventors have shown that Y201 MSCs produce abundant, consistent EVs for which the size, morphometric, EV marker, miRNA and proteomic data has been collected. Advantageously, the inventors have demonstrated that Y201 EVs stimulate cell growth (donor MSCs and cartilage cells from arthritis patients), cartilage formation and suppress inflammation. Surprisingly, further engineering of the Y201 MSCs using CRISPR/Cas9 to target a deletion of the FGFR3 gene increases proliferation, migration activity and secretory behaviour to enable growth in serum-free conditions.

Some of the characteristics of the Y201 cell lines and derivatives claimed herein have previously been described in the literature (James et al., 2015; Kay et al., 2022). The cell lines themselves are now disclosed herein for the first time.

### Methods

### Y201 Cells and Y201-EVs

### Y201 Cells

Y201 MSCs were generated by immortalisation with human telomerase reverse transcriptase (hTERT) and characterised as previously described (James et al, 2015, Kay et al, 2022).

### Cell Culture and Preparation of EV Depleted Medium

Dulbecco's Modified Eagle's Medium (DMEM) (Cat: 41966, ThermoFisher Scientific) supplemented with 20% Foetal Bovine Serum (FBS) and 1% Penicillin/Streptomycin (P/S) (Cat: 15140122, ThermoFisher Scientific) was centrifuged at 10,000 g for 18 hrs at 4 °C to deplete the medium of serum-derived bovine EVs. During cell expansion, medium was diluted in equal volumes with DMEM supplemented with 1% P/S. Y201 cells and the comparator MSC line, MSC #2, were grown at 37 °C in 5% CO₂/95% air atmosphere. Human articular chondrocytes (AC) were isolated from primary donors following fully informed ethical consent (LREC 07/Q1105/9). ACs were cultured in DMEM-F12 supplemented with 10% FBS and 1% P/S.

### Conditioned Media Collection for Secretome Analysis and Functional Assays

Conditioned media was collected from 2×T175 flasks of Y201 and MSC #2 lines. Cells were grown to ~80% confluency before washing 2× with PBS, 17 ml of serum-free DMEM was added to the flasks and incubated at 37 °C and 5% CO₂ for 24 h. Media was collected and then centrifuged at 300 g to remove any large cell debris. For functional assays, medium was stored at −80 °C until required. For proteomic analyses, the medium was concentrated in 3 kD MWCO tubes (GE Healthcare) at 4500 g until concentrated to ~1 ml in volume. Media were stored at −80 °C until required.

### Ptychography, Cell Tracking and Image Analysis

Ptychography was performed using a PhaseFocus VL21 Livecyte imaging platform for live cell tracking analysis. Images were taken at 20-26-minute intervals dependent on colony location for 96 hours from 4 days post-seeding. Cell morphology and migration was quantified using the PhaseFocus analysis platform and statistical tests performed in Graphpad Prism. Rose plots were generated using the mTrackJ plugin in ImageJ. The image analysis programme CellProfiler was used to generate a pipeline to assess the morphological characteristics of MSCs.

### Colony Forming Assays and Image Analysis

For colony forming unit-fibroblast (CFU-F) assays, all cell types were seeded at 10 cells/cm² in 6-well plates using DMEM supplemented with 20% Hyclone FBS containing 100 units/ml penicillin, 100 μg/ml streptomycin. Conditioned medium for use in the CFU-F assays was collected from Y201 and MSC #2 cells by incubating in serum-free media at ~80% confluency for 24 hours before collecting media, centrifuging at 300 g to remove cell debris, and counting the number of cells. The conditioned medium was then diluted with additional serum-free DMEM to give 12 ml conditioned media/million cells. This medium was then supplemented with a final concentration of 20% Hyclone FBS for use in CFU-F assays. For CFU-Fs primary cells and cell lines were seeded in unconditioned Hyclone medium before media changes were performed every 4 days post-seeding and plates were fixed and stained at day 10 for cell lines and day 14 for primary cells. Plates were stained with (0.05% crystal violet+1% formaldehyde+1% methanol in PBS) for imaging or were washed 1× with PBS and the cells lysed with 350 μL of RA1 cell lysis buffer+3.5 μL β-mercaptoethanol for every 3 wells. Lysates were stored at −80 °C for subsequent qRT-PCR analysis. Well plates were air dried before scanning on an Epson Perfection 4990 Photo scanner at 1200 dpi. A CellProfiler pipeline was subsequently developed to detect and measure colonies accurately.

### Focal Adhesion Assessments

Cells were plated onto glass coverslips at low density and left to adhere for 24 h. Cells were fixed briefly in 4% methanol-free PFA in PBS before washing 3× with PBS. Cells were permeabilised in 0.1% Triton X-100 in PBS for 30 minutes and washed 3× with PBS. Cells were then blocked for 30 minutes with 10% goat serum in PBS. Anti-vinculin antibody (1:400 dilution) was added in 1% BSA and incubated at room temperature for 1 hour. Cells were washed 3× with PBS before Goat anti-mouse and Alexafluor 488 conjugated secondary antibody (1:2300, ThermoFisher) was added along with Cruzfluor 594 conjugated phalloidin (1 in 1000, Santa Cruz) for 45 minutes in PBS followed by another 3× washes. Nuclei were counterstained with 0.2 μg/ml DAPI for 10 minutes before rinsing briefly in distilled dH2O water and leaving to air-dry. Coverslips were mounted onto a microscope slide with Prolong gold antifade (ThermoFisher).

Slides were imaged on a Zeiss LSM880 or LSM780 microscope. Focal adhesion sizes were quantified using ImageJ.

### Gene Set Enrichment Analysis (GSEA)

Lists of gene names of significantly upregulated proteins or targets of enriched miRNAs were collated and assessed using the Broad Institute GSEA Molecular Signatures Database (V7.0). Lists were analysed against the KEGG Pathway database for significant enrichment.

### EV Isolation by Sequential Differential Ultra-Centrifugation

5×10⁵ Y201 cells were seeded in T175 flasks with EV depleted medium up until reaching 80-90% confluence. Medium was aspirated and cells were washed thrice with PBS and serum free medium was added. Two medium collections were performed every 24 hrs and stored in −70 °C for EV isolation, at the end of the second collection cells were counted. EVs were isolated according to the Thery et al protocol (see Théry, Clotilde, Sebastian Amigorena, Graça Raposo, and Aled Clayton. 2006. “Isolation and Characterization of Exosomes from Cell Culture Supernatants and Biological Fluids.” Current Protocols in Cell Biology/Editorial Board, Juan S. Bonifacino . . . [et al.] Chapter 3) with minor modifications, all steps were performed at 4 °C. Conditioned medium were centrifuged at 300 g for 5 mins to remove dead cells and debris followed by a second spin at 2000 g for 20 mins. Supernatant was transferred to Ty45i thick-walled ultracentrifuge tubes (Cat: 355655, Beckman-Coulter) and centrifuged at 10,000 g for 45 mins in L100-XP Beckman-Coulter ultracentrifuge. The 10K fraction was collected by vigorous re-suspension with PBS and transferred into thick-walled micro-ultracentrifuge tubes (Cat: 357448, Beckman-Coulter). Supernatant was centrifuged again at 100,000 g for 90 mins and the 100K fraction was collected into thick-walled micro-ultracentrifuge tubes. 10K and 100K fractions were centrifuged at 10,000 g and 100,000 g in a Beckman-Coulter TL100 μltracentrifuge for 45 and 90 mins respectively. EV pellets were re-suspended in PBS and the EV suspensions were transferred in protein LoBind tubes.

### Concentration and Size Range of EVs Using Nanoparticle Tracking Analysis (NTA)

The concentration and size of EVs were determined using the NTA3.4 software, videos were obtained using the Nanosight LM14 equipped with a green laser (532 nm). The 10K and 100K Y201 EV fractions were diluted in PBS to a concentration of 20-120 particles per frame. A script was used obtaining 5×60 s video recordings of all events for further analysis. The experimental conditions were as follows: i) Measuring time: 5×60 s, ii) Blur: Auto, iii) Detection Threshold: 4-5, iv) Blur size: Auto, iv) Number of frames: 1499.

### Transmission Electron Microscopy (TEM)

10 μl of EV suspension were deposited on a 200 mesh copper grid with a formvar/carbon support film and left for 3 mins to air dry. The grids were washed with three drops of dH2O to remove the salts from the PBS. Negative staining was the attained by adding 10 μl of 1% uranyl-acetate

### Western Blotting

Cell lysates and EVs were separated on a 12% SDS-PAGE gel. The separated proteins were transferred onto a nitrocellulose membrane using the iBlot2 dry blotting system (20V for 1 min, 23V 4 mins, 25V 2 min). Membranes were blocked for an hour with 5% Bovine Serum Albumin (BSA) in PBST, the membranes were incubated with the following mouse monoclonal antibodies: i) Flotillin-1 (1:500; SantaCruz: 133153), ii) CD63 (1:1000; SantaCruz: 365604), iii) CD81 (1:1000; SantaCruz: 23962), iv) Alix (SantaCruz: 166952), v) GRP78 BiP (Abcam: 21685) vi) MFG-E8 (1:200; proteintech: 67797-1-Ig) for an hour at room temperature or overnight at 4 °C. After the incubation, membranes were incubated with anti-mouse horseradish peroxidase-conjugated secondary antibodies in 1:1500 dilution. The protein bands were visualized using the iBright western blot imaging system and the ECL PicoPlus Chemiluminescent substrate (Cat: 34577, ThermoFisher Scientific).

### Proteomic Analysis of Y201 Whole Secretome and Y201 EVs

Concentrated whole secretome samples were added to 8M urea with 20 mM HEPES, 1 mM sodium orthovanadate, 1 mM β-glycerophosphate and 2.5 mM sodium pyrophosphate. Protein was in-solution reduced and alkylated before digestion with a combination of Lys-C and trypsin proteases. Resulting peptides were analyzed over 1 h LC-MS acquisitions using an Orbitrap Fusion. Peptides were eluted into the mass spectrometer from a 50 cm C18 EN PepMap column. Three biological replicates for each cell line were run. Tandem mass spectra were searched against the human subset of the UniProt database using Mascot and peptide identifications were filtered through the Percolator algorithm to achieve a global 1% false discovery rate (FDR). Identifications were imported back into Progenesis QI and mapped onto MS1 peak areas. Peak areas were normalized to total ion intensity for all identified peptides. Relative protein quantification was performed using relative peak areas of non-conflicting peptides. Relative fold differences and associated p-values for differential abundance were calculated in Progenesis QI.

EVs isolated from 8×T175 of cells were added to 8M urea with 20 mM HEPES and a phosphatase inhibitor cocktail comprising 1 mM sodium orthovanadate, 1 mM β-glycerophosphate and 2.5 mM sodium pyrophosphate. Protein was in-solution reduced and alkylated before digestion with a combination of Lys-C and trypsin proteases. Resulting peptides were analysed over 1-hour LC-MS acquisitions using an Orbitrap Fusion (Thermofisher). Peptides were eluted into the mass spectrometer from a 50 cm C18 EN PepMap column. Three biological replicates for each cell line were run. Tandem mass spectra were searched against the human subset of the UniProt database using Mascot and peptide identifications were filtered through the Percolator algorithm to achieve a global 1% false discovery rate (FDR). Identifications were imported back into Progenesis QI and mapped onto MS1 peak areas. Peak areas were normalised to total ion intensity for all identified peptides. Relative protein quantification was performed using relative peak areas of non-conflicting peptides. Proteins were accepted for analysis provided they were detected with ≥2 peptides and ≥1 unique-peptide in at least one sample. Relative fold differences and associated p-values for differential abundance between pairwise comparisons of cell lines was calculated in Progenesis QI by ANOVA.

Analysis of EV microRNAs

EVs from Y201 cells were thawed and allowed to reach room temperature. RNA was extracted using the Total Exosome RNA and Protein Isolation kit (Invitrogen) following manufacturer's instructions. Briefly, samples were thawed and diluted with 1×PBS to 200 μL total volume in an RNAse-free tube. 200 μL of pre-warmed denaturing solution was then added and mixed before incubating samples on ice for 5 minutes. 400 μL of Acid-Phenol: Chloroform was then added to each sample and they were vortexed for 60 seconds before centrifuging at 13,000 g for 5 minutes at room temperature. The aqueous (upper) phase was then transferred to a fresh RNAse-free tube and the volume recovered was recorded and then used in purification. The aqueous phase was diluted with 1.25× volumes of 100% ethanol and mixed. The aqueous-phase/ethanol mix was loaded onto a filter cartridge in a fresh tube and centrifuged at 10,000 g for 15 seconds. The flow through was discarded and the cartridge was then washed by centrifuging with 700 μL of miRNA wash solution 1 at 10,000 g for 15 seconds. Flow through was discarded and then 2× washes with 500 μL of miRNA wash solution 2/3 were performed at the same settings. The cartridge was dried with a spin of 10,000 g for 1 minute. The cartridge was then removed and placed into a fresh collection tube and the RNA was eluted using 50 μL of pre-heated elution solution and centrifuging at 10,000 g for 30 seconds. The eluate was then passed through the cartridge a second time to improve yield. After RNA purification the samples were concentrated using Amicon Ultra 0.5 ml centrifugal filters (Sigma-Aldrich) by first topping up to 400 μL with RNAse-free water. Samples were centrifuged at 14,000 g for 88 minutes before inverting and collecting RNA in a fresh tube at 8000 g for 2 minutes. Total RNA was then quantified on a Bioanalyzer 2100 (Applied biosystems) using a Pico chip. 5-3 μL of RNA was used in the NanoString miRNA ligation reaction. NanoString was performed following manufacturer's miRNA sample preparation protocol using the nCounter Human v3 miRNA expression assay codeset (NanoString). miRNA counts were normalized using spike-ins from the NanoString procedure before miRNAs with >20 counts in ≥1 sample were filtered for analysis. Comparisons of EV miRNAs was performed in the R programming language.

### CyQuant Proliferation Assay

Cells were seeded in a 96-well plate at a 9,000 cell/cm² density and left to adhere overnight. The following morning, MSC #2 cells were treated with Y201 EVs at 1×, 5× and 10× concentrations. The treatments were calculated using the following equation: (Ni/Nt)/v× where Ni is the number of donor cells, Nt number of recipient cells, v is the volume of the EV suspension and X is the number of the desired treatment. Cells were subjected to daily EV treatments and microplates were frozen down. To assess the proliferative effect of the EVs the CyQuant proliferation assay kit (Cat: C7026, Invitrogen) was used. Working CyQuant solution was prepared by diluting the cell-lysis buffer stock solution in 20-fold using dH2O and the CyQuant GR stock solution was added in a 400-fold for 5-10 mins. The quantification of the cellular DNA per well was achieved by saking the microplates for 2 mins at 300 rpm and measuring the fluorescence at 480/502 nm (excitation/emission) using the CLARIOstar plate reader.

### Proliferation Assay Using the LiveCyte Microscope

Cells were seeded in 24-well plates at a 4,500 cell/cm² density and left to adhere overnight. MSC #2 cells were treated with Y201 EVs at a 10× concentration. The microplates were loaded onto the LiveCyte microscope and cell proliferation was monitored for 72 hrs using the quantitatively ptychographic phase imaging technique and analysed using the phasefocus software v3.5.

Articular chondrocytes (ACs) isolated from OA donors, were seeded in a 24-well plates at a 3,000 cell/cm² and left to adhere overnight. Cells were treated with Y201 100K EVs at a 10× and 20× concentrations and cell proliferation was monitored for 72 hrs on a LiveCyte microscope as above.

### Inhibition of EV Cargo Integrin Mediated Transfer to Recipient Cells

AC cells were seeded in 24-well plates at a 3,000 cell/cm² and left to adhere overnight at 37 °C, 5% CO2. For inhibition studies, cells were treated with 200 μM Gly-Arg-Gly-Asp-Ser-Pro (GRGDSP—SEQ ID NO: 16) or Gly-Arg-Ala-Asp-Ser-Pro (GRADSP—SEQ ID NO. 17) peptides (Sigma-Aldrich, SCP0157, SCP0156) for 6 hrs. Cells were washed twice with PBS and Y201 100K EVs were added at a 20× treatment. Proliferation rate was monitored for 72 hrs using the LiveCyte microscope.

### Scratch Assay

MSC #2 cells were seeded in 24-well plates at a 30,000 cells/cm² density and left to adhere for 6-8 hrs. Cells were washed with PBS and cells were serum starved overnight. To mimic a wound, a 10 μl pipette tip was used to create a scratch in the confluent monolayer of cells and washed with PBS to remove dead cells and debris. MSC #2 cells were treated with 10×Y201 100K EVs and the closure of the wound was monitored over 24 hrs using the quantitatively ptychographic phase imaging technique with the LiveCyte microscope. Images were analysed using Phasefocus Software v3.5 Single cell analysis was performed using the MTrackJ plugin in ImageJ and measurements were imported into the chemotaxis tool to calculate the cell velocity, total track length, Euclidean distance, directness and forward migration index.

### Chondrogenesis

Primary MSCs were isolated from femoral head donations from three osteoarthritis patients following ethical approval (LREC 07/Q1105/9). Prior to formation of test condition micromass pellets, 2.35×10⁵ MSCs per test were exposed for 6 hours to EVs derived from 2.35×10⁶ Y201 MSCs. Following this, micromass pellets were formed at a density of 2.35×10⁵ cells per pellet in 100 μl growth media in microcentrifuge tubes by centrifuging at 300 g for 5 minutes and then incubating overnight at 37 °C.

Pellets were detached and growth medium was initially topped up to 1.2 ml taking care not to disrupt the pellet. Negative controls received basal medium comprising DMEM containing sodium pyruvate and L-glutamine, supplemented with 1% ITS+3, 40 μg/ml L-Proline, and 1% non-essential amino acids. Chondrogenic medium comprised basal medium with the addition of 0.1 μM dexamethasone, 50 μg/ml L-Ascorbic Acid and 10 ng/ml TGF-β1.

Tubes were incubated at 37 °C and medium changed every 3-4 days. Test conditions were treated once per week with EVs derived from 2.35×10⁶ Y201 MSCs. At days 0, 7, 14 and 21 pellets were removed and rinsed twice with 1 ml PBS then fixed for 10 minutes with cold paraformaldehyde. Pellets were again washed twice with PBS and paraffin wax embedded within 48 hours of fixation using a Leica tissue processor on the small biopsy program. Pellets were then sectioned at 5 μm thickness. Paraffin embedded sections were cleared and rehydrated prior to staining with 0.02% Fast Green for 5 minutes and 0.1% Safranin O for a 15 minute period. Following staining, sections were dehydrated and mounted using DPX mounting medium prior to imaging at 20× resolution with Z-stack imaging on an Axio Scan.Z1 slide scanner.

### T Cell Activation Assay

MSC-derived EV immunomodulation for deactivation and suppression of T cell proliferation Suspension cultures of 1.0×10⁵ primary human peripheral blood-derived CD4+ T cells (Stem Cell Technologies) were pre-treated for 6 hours with EVs isolated from serum-free conditioned medium from 2.0×10⁶ of Y201 MSCs collected over 24 hours of culture. For positive controls, 1.0×10⁴ Y201 MSCs were seeded into a 96-well U-bottomed plate and cultured for 24 hours at 37 °C, 5% CO₂ prior to addition of T cells.

Continual proliferative capacity was assessed as a measure of T cell proliferation. CD4+ T cells were stained for 10 minutes at 37 °C using 1 μM VPD450 Violet proliferation dye (eBioscience, Inc.). T cells were activated using anti-CD38/CD28 Dynabeads (Thermo Fisher) at a bead-to-cell ratio of 1:1 then seeded at a density of 1.0×10⁵/well (ratio 10:1) in 200 μl RPMI-1640 with 10% FBS, 0.05 μg/mL IL-2 (Peprotech, Inc). T cells seeded alone (no treatment) or seeded onto 1.0×10⁴ Y201 MSCs were applied as negative and positive controls respectively and all conditions were tested with and without activation. Plates were cultured for 6 days at 37 °C prior to removal of Dynabeads with the DynaMag-2 as per manufacturer's recommendations. T cell proliferation was assessed with flow cytometry, with reduction in signal intensity visualised for peaks using FCS Express 7.0 proliferation analysis. Proliferation was assessed through VPD450 dilution (diminished staining intensity) described through a proliferative index (PI) calculated from the fluorescence intensity at each cell division as described previously (Kay et al, 2022). Proliferative cycles undertaken were calculated on 50% fluorescence intensity reduction peaks, measuring from fluorescence intensity of the first division and the final division detected.

### MSC Immunomodulation to Direct Effector T Cell Polarisation

For assessment of T helper differentiation, T cells were activated and cultured with MSC-derived EVs or with MSC monolayers, as described above. The following reagents and antibodies for reactivation, transport inhibition and staining were sourced from eBioscience. Following 6 days of culture, T cells were re-stimulated using a combination of phorbol 12-myristate 13-acetate (PMA) (50 ng/ml) (Sigma Aldrich) and lonomycin (1 μg/ml) (Invitrogen) and intracellular cytokines retained using transport inhibitor cocktail with 10 μg/ml brefeldin A and 2 μM Monensin (Invitrogen). T cells were cultured for 4 hours at 37 °C then stained for surface marker CD4. Intracellular staining for CD4+ helper T cells was undertaken for anti-human IFN-γ (Th1), IL-4 (Th2) or IL17a (Th17) or CD4 and CD25 followed by fixation/permeabilisation and staining for nuclear protein FOXP3 for regulatory T cells. All cells were measured using the Cytoflex LX flow cytometer and analysed with FCS Express 7. Comparisons were drawn for percentage of T helper differentiation within the CD4+ cell population and signal intensity (Median) for each antibody tested.

### In Vivo Assessment of Immunomodulatory Capacity of Y201 MSCs in a Murine Peritonitis Model

An in vivo peritonitis model was used in C57BL/6J mice aged 8-10 weeks with zymosan and schistosome egg irritant for induction of inflammation. These experiments were carried out in accordance with the Animals and Scientific Procedures Act 1986, under UK Home Office Licence (project licence number PPL PFB579996 approved by the University of York Animal Welfare and Ethics Review Board). At day 0, mice were administered with an intraperitoneal infusion of 1 mg of zymosan A (Merck) or 5000 schistosome eggs in 200 μl of PBS. Immediately following administration of irritant, test condition mice were administered an intraperitoneal infusion of EVs isolated from serum-free conditioned media collected over 24 hours from either 4.0×10⁷ of Y201 MSCs in 100 μl of PBS to treat zymosan-induced inflammation or 2.0×10⁷ of Y201 in 100 μl of PBS to treat schistosome eggs-induced inflammation; negative control mice were given PBS vehicle only.

After 24 hours, mice were euthanised using CO2 overdose and cervical dislocation. Intraperitoneal injection of 4 ml of ice cold RPMI-1640 was administered as peritoneal lavage. The process was repeated with a second 4 ml RPMI-1640 wash and wash solutions pooled to form the peritoneal exudate cells (PEC). For each animal tested, red blood cells were lysed from the PEC using Red Cell Lysis buffer (Merck) and a cell count performed. PEC samples were initially stained for Ly6C (APC), Ly6G (FITC), F4/80 (PE-Cy7) CD45 (PerCP-Cy5.5) (BioLegend) and Ly6G (FITC), CD11b (BUV395) and SiglecF (BV421) (BD). PEC samples were then stained for TCRb (AF488), CD3 (APC-Cy7), CD4 (PerCP-Cy5.5), CD62L (APC) and CD44 (PE) (BioLegend). For all tests, Zombie Aqua (BioLegend) was used to exclude dead cells.

### Statistical Analysis

The statistical analyses for all experiments were performed using GraphPad Prism v9.0.2. The statistical significance between my treatment and control (DMEM) was assessed by T-test and Two-Way ANOVA and Bonferroni corrections. n=3, error bars are SEM and asterisks represent the following P values, *p<0.05, **p<0.01, ***p<0.001, ****p<0.0001

### Fibroblast Growth Factor Receptor 3 (FGFR3) Knockout Cells

Generating FGFR3 Cell Line with CRISPR/Cas9

Single guide RNA (sgRNA) structures were designed using the CRISPR design tool (crispr.mit.edu). Potential sequences were ranked by the programme based on specificity and off-target effects, sequences were chosen that possessed the fewest off-target effects. The sgRNA sequences selected are below: (5′-3′)

sgRNA Fwd:
(SEQ ID NO: 1)
CACCGCATCCGGCAGACGTACACGC
sgRNA Rev:
(SEQ ID NO: 2)
AAACGCGTGTACGTCTGCCGGATGC

A single stranded oligonucleotide (ssODN) was designed to introduce a specific base mutation in the FGFR3 sequence by homologous recombination. The ssODN was designed so the double stranded break introduced by Cas9 was within 100 base pairs of the mutation and to avoid the Cas9 enzyme targeting the ssODN, the antisense of the wanted sequence was used. The ssODN sequence is below: (5′-3′)

ssODN:

(SEQ ID NO: 3)
CAGCACCGCCGTCTGGTTGGCCGGCAGCCCCGCCTGCAGGATGGGCCGGT
GCGGGGAGCACTCCAGCACGTCCAGCGTGTACGTCTGCCGGATGCTGCCA
AACTTGTTCTCCACGACGCA

All primers were ordered from Integrated DNA Technologies and purified via desalting.

Y201 hTERT MSCs were electroporated, using previously optimised parameters, and serial dilution used to isolate individual colonies as described elsewhere herein. Y201 hTERT MSCs were used as a WT control throughout results to compare to the CRISPR/Cas9 treated cells, these are referred to as WT hTERT MSCs herein.

### CRISPR/Cas9 On-Target and Off-Target Sequencing

PCR amplification was performed as described elsewhere herein and products sent for Sanger sequencing using the unique forward primer at a 3.2 μM concentration. The primers used for amplification and sequencing are shown in Table 1.

TABLE 1
Primers used in on-target and off-target sequencing

| Primer Target | Forward Primer (5′-3′) | Reverse Primer (5′-3′) |
|---|---|---|
| FGFR3 CRISPR/Cas9 | CAC GGC CCA GCT CTG AGA | ACC CAA ATC CTC ACG |
| Validation | AAG (SEQ ID NO: 4) | CAA CC (SEQ ID NO: 5) |
| FGFR3 Offtarget 1 | CAT CAA GCC ATC CAC TAT | CTA CAC TGT CCA CCA |
| | ACA G | TAC |
| | AGG CGT CTG GGA GAC ATA | TTA TGC TGG ATG TAT |
| | CA (SEQ ID NO: 6) | GGG GCT (SEQ ID NO: 7) |
| FGFR3 Offtarget 2 | CCT GTC CTC CAT TCA CCC | GAG TTC AGT GAA GGG |
| | ACA C (SEQ ID NO: 8) | GAG CC (SEQ ID NO: 9) |
| FGFR3 Offtarget 3 | CTG AGA AAG AGG TCA GGA | CTG CTA GAG CAG GAG |
| | G (SEQ ID NO: 10) | TGA GG (SEQ ID NO: 11) |
| FGFR3 Offtarget 4 | CGG GTC CCA CTC CTA GAC | CGG CCC TTA CCG ATA |
| | AC (SEQ ID NO: 12) | CTT CAT (SEQ ID NO: 13) |
| FGFR3 Offtarget 5 | CAA TAA ACG TTG GGT GCC | CTC AGT AAG TGC TGG |
| | GC (SEQ ID NO: 14) | CCT CTG (SEQ ID NO: 15) |

### Population Doubling Time Calculations

Population doubling time was determined by seeding equal numbers of WT and FGFR3-KO MSCs in T25 flasks, and counting cell number each day for 4 days. Cells were counted using Countess II Automated Cell Counter (Invitrogen) using trypan blue cell exclusion dye to discount dead cells. Doubling time was calculated using the formula: t*log (Nf/Ni)

Where t is time in days between counts, Nf is the final number of cells counted, and Ni is the initial number of cells.

For cumulative population doublings, cells were counted throughout 50 days of continuous culture, and the number of population doublings between each count was calculated using the formula: log (Nf/Ni)

Where Nf is the final number of cells counted, and Ni is the initial number of cells seeded. 500,000 cells were reseeded after each count for both WT and FGFR3-KO MSCs.

### Morphological Analyses

### Cell Seeding and Treatments

WT and FGFR3-KO cells were seeded in a 24 well plate at 4000 cells per cm² and allowed to adhere overnight before washing with PBS and staining with crystal violet.

### Phalloidin Staining of the Actin Cytoskeleton

WT and FGFR3-KO cells were seeded on 10 mm circular glass coverslips in 24 well plates at 4000 cells per cm² and allowed to adhere overnight. Coverslips were washed with PBS and fixed with 4% paraformaldehyde for 20 minutes at 37 °C and 5% CO₂. Cells were washed again with PBS and treated with Alexa Fluor 594 Phalloidin (Invitrogen) at 1:1000 dilution in PBS; for 1 hour in the dark at room temperature. Cells were washed three times with PBS then counterstained with DAPI for 5 minutes before a further three PBS washes. Coverslips were mounted onto slides with Vectashield Mounting Medium (Vector Laboratories), and sealed with nail varnish before imaging the following day with a Zeiss 710LSM confocal microscope.

### Anti-Arp3 Immunofluorescence

Cells were seeded and fixed as described elsewhere herein, then permeabilised with 0.1% Triton X-100 for 30 minutes before washing three times with PBS. Cells were blocked with 1% bovine serum albumin for 1 hour, before Anti-Arp3 (Sigma) was applied at 1:200 dilution in 1% BSA for 2 hours at room temperature. Cells were washed in PBS. Goat anti-mouse Alexa Fluor 647 conjugated secondary antibody (Thermofisher) was applied at 1:200 alongside Alexa Fluor 594 Phalloidin at 1:1000 in PBS for 1 hour, at room temperature in the dark. Cells were washed in PBS and counterstained with DAPI, mounted and imaged as described elsewhere herein.

### Characterisation of Extracellular Vesicles (EVs)

### Conditioned Media Collection and EV Isolation

FBS-EV depleted media was generated by centrifugation of DMEM containing 20% FBS and 1% P/S at 100,000 g for 18 hours at 4 °C, using a Ty45i rotor in a Beckman Coulter Optima L-100XP Ultracentrifuge. This FBS-EV depleted media was then diluted with serum-free media containing 1% P/S, to a final concentration of 10% FBS.

WT and FGFR3-KO cells were seeded at 500,000 and 400,000 cells respectively per T175 flask in FBS-EV depleted media and cultured until reaching approximately 90% confluency. Cells were washed three times with PBS, and FBS-EV depleted media was replaced with serum-free media. After 24 hours, media was collected, fresh serum free media added, and collected after a further 24 hours. This conditioned media (CM) was stored at −70 °C.

CM was centrifuged at 300 g for 5 minutes to remove cells and debris. The supernatant was either used for CM treatments or to isolate EVs. For EV isolation, the media was centrifuged at 2000 g for 20 minutes, the supernatant transferred into Ty45i tubes, and the pellet resuspended in PBS. The resuspended pellet was transferred into a protein low-binding tube and centrifuged again at 2000 g for 20 minutes. The supernatant was discarded and the pellet is the isolated 2k fraction. The transferred supernatant from the first 2000 g spin was centrifuged at 10,000 g for 45 minutes, and the new supernatant transferred into fresh Ty45i tubes. The pellet was resuspended and transferred into micro-ultracentrifuge tubes, and centrifuged in a TLA 100.3 rotor at 10,000 g for 45 minutes to isolate the 10k fraction. The transferred supernatant from the first 10,000 g spin was centrifuged at 100,000 g for 90 minutes and the supernatant kept (for use as EV-negative media). The pellet was resuspended, transferred to micro-ultracentrifuge tubes, and centrifuged again at 100,000 g for 90 minutes to isolate the 100k fraction. All pellets were thoroughly resuspended in particle-free PBS, and all centrifugations were performed at 4 °C.

### Nanoparticle Tracking Analysis

Nanoparticle tracking analysis (NTA) was conducted using Nanosight NTA Software 3.4 Build 3.4.003 (Malvern Panalytical). Images were captured using an sCMOS camera and 532 nm laser wavelength.

### Transmission Electron Microscopy

EV isolation was performed as described elsewhere herein, except instead of resuspending the final pellets in PBS, pellets were resuspended in 2% PFA and kept at 4 °C overnight. EVs were then placed on glow discharged Formvar/Carbon-coated grids for 20 minutes, before fixation with 1% glutaraldehyde for 5 minutes. Grids were washed eight times with PBS, then incubated with 2% uranyl acetate for 5 minutes in the dark. Methylcellulose was centrifuged at 100,000 g for 95 minutes before grids were incubated in methylcellulose-uranyl acetate for 10 minutes on ice. Excess liquid was drawn off on filter paper and samples allowed to dry before imaging.

### Migration Assays

### Scratch Wound Assays

WT and FGFR3-KO MSCs were seeded at 42,500 cells per cm² in a 48 well plate and allowed to adhere overnight. Wells were checked for confluency and even distribution of cells before washing with PBS and replacing media. A 200 μl pipette tip was used to generate a scratch, ensuring even, consistent pressure was applied across the well, before washing again with PBS and replacing fresh media. Cells were imaged 0 and 24 hours after scratching, with marks made on the plate to ensure the same field of view was captured. The resulting images were overlaid to mark the initial size of the wound on every image in each field of view. ImageJ was then used to quantify the initial and final wound area, and the percentage healing was calculated.

### Ptychography Analysis

Cells were seeded in an Ibidi Culture-Insert Plate as per manufacturer's instructions, and allowed to adhere overnight before inserts were removed, creating a space in the centre of each well for cells to migrate into. Plates were imaged using Livecyte microscopy (Phasefocus), for 24 hours, kept at 37 °C and 5% CO₂. Images were captured every 5 minutes and analysed using Cell Analysis Toolbox (CAT) Software (Phasefocus). The software segmented cells using an advanced fuzzy threshold algorithm to track individual cell metrics.

### Secretome-Treated Scratches

Cells were seeded and scratched as described elsewhere herein. Treatments were applied immediately following scratching. Control wells were treated with DMEM supplemented with 1% P/S and, where specified, 10% FBS. CM, EVs and EV-ve media was collected as described elsewhere herein. A 1× treatment dose of EVs was calculated using the equation:

$$( \frac{{n}_{i}}{{n}_{t}} ) v$$

Where Ni is the number of cells EVs were isolated from, Nₜ is the number of cells receiving the treatment, and v is the volume EVs were resuspended in. Individual cell metrics were collected using Livecyte microscopy, with images taken every 30 minutes for 24 hours, before analysis using CAT software. For subsequent CM-treated scratches, analysis was performed in ImageJ.

### RNA-Seq Analysis

RNA Isolation, cDNA Library Preparation and Sequencing

Total RNA for transcriptome analysis was isolated by centrifuging Y201 and FGFR3 KO MSCs for 5 minutes at 400 g, before resuspension in 350 μL of RA1 lysis buffer (Nucleospin RNA II kit) and 3.5 μL of β-mercaptoethanol. Following lysis, RNA was extracted using the Nucleospin RNA II columns (Macherey-Nagel, Germany) following manufacturer's instructions. Briefly, lysates were cleared first by passing through the column before conditions were adjusted for optimal RNA binding by mixing with 350 μL of 70% ethanol. RNA was then bound to the column and the column desalted using membrane desalting buffer. The column was then treated with DNAse I for 15 minutes and the column was washed and dried to remove the presence of buffer. RNA was then eluted in 30 μL of RNAse-free H₂O and quantified spectroscopically using a Nanodrop. RNA quality was assessed using an Agilent 2100 Bioanalyzer. mRNA was then captured using Oligo-dT beads which bind to the poly-A tails found specifically on mRNA, thus removing other RNA species such as ribosomal RNA which would contaminate the sample. mRNA was then fragmented and these fragments were used as templates for cDNA synthesis, which were then ligated to adapters. This library was then amplified and sequenced using a HiSeq 2500 sequencer.

### Read Mapping

The STAR splice-aware read mapper (https://github.com/alexdobin/STAR) was used to map reads, with following options: “--outSAMstrandField intronMotif”, “--outFilterType BySJout”, “--outFilterIntronMotifs RemoveNoncanonical” and “--outSAMtype BAM SortedByCoordinate”. Reads were mapped against the pre-computed indexed GRCh38 Gencode 24 version of the human genome provided by the STAR authors.

### Quantitation and Differential Expression Analysis

Cufflinks (http://cole-trapnell-lab.github.io/cufflinks/) was used to quantify mapped reads and perform differential expression analysis. The associated GTF annotation file from Gencode version 24 was provided with the “-g” option and cDNA fasta sequences with the “-b” option. The resulting GTF files, from Cufflinks, were merged with Cuffmerge and used with Cuffdiff for differential expression analysis.

### RNA-Seq Analysis

RNA-seq data yielded a list of differentially expressed transcripts between WT and FGFR3-KO MSCs, defined as those with a log 2 fold change of >+1 and an adjusted p value of <0.05. Transcripts were then divided into two gene lists corresponding to: upregulation or downregulation in FGFR3-KOs versus WT, and those which had <5FPKM reads in both cell lines were excluded. GO term analysis was performed in STRING (Szklarczyk et al., 2019), and KEGG pathway analysis conducted using Enrichr (Chen et al., 2013) and KEGG Mapper (Kanehisa and Sato, 2020). Data was exported and graphed using ggplot2 (Wickham, 2016) in RStudio (RStudio Team, 2020).

### Alamar Blue Cell Viability Assay

WT and FGFR3-KO cells were seeded in 96 well plates, with one plate per timepoint, and allowed to adhere overnight. Alamar blue cell viability agent (Invitrogen) was then added to the day 0 plate, in fresh media at a final concentration of 10%. After 4 hours, fluorescence was measured using a Clariostar plate reader. For all experiments, treatments were applied on day 0 and alamar blue readings were taken at the same time each day for a further 3 days. For initial comparison of WT and FGFR3-KOs, cells were plated at 9375 cells per cm², and for all subsequent experiments were plated at 7812 cells per cm² to ensure cells did not reach full confluency before the end of the time course. Day 2 alamar blue plates were used for morphological analysis of CM-treated and U0126-treated MSCs, by washing the plates with PBS after the fluorescence readings were taken, and staining with crystal violet. All results were normalised to the fluorescence of control treatments at day 0, and reported as relative fluorescence.

### Additional Methods

### Y201 EV Proteomic Gene Ontology Term Enrichment and Clustering

Gene Ontology (GO) enrichment was performed using the ClueGO plugin for the Cytoscape software package (Bindea et al. 2009; Shannon et al. 2003). Gene lists were assessed for enrichment against the Biological Process and Molecular function GO genesets with Benjamini-Hochberg False Discovery Rate (FDR) corrected p-values. Redundancy of GO terms was reduced by using the GO-fusion setting in ClueGO before automated clustering of significant GO terms (FDR-corrected p<0.05). Cluster diagrams were generated from ClueGO results using the AutoAnnotate plugin to facilitate organisation and labelling of like-terms and to generate titles for clusters based upon common words (Kucera et al. 2016). Clusters were moved to aid visualisation.

### CFSE-EV Labelling

EVs were isolated by differential ultracentrifugation and labelled using carboxyfluorescein diacetate succinimidyl-ester (CFSE) (Invitrogen, Cat: C34554). EVs were incubated with 20 μM CFSE for 120 mins at 37 °C in a final volume of 300 μl PBS. Labelled EVs were pooled by ultracentrifugation at 100,000×g for 90 mins and resuspended in PBS to remove the unbound CFSE dye. Labelled EVs were transferred in a protein LoBind Eppendorf tube for storage.

### Analysis of EV Uptake by Flow Cytometry

MSC #2 cells were detached from plastic using 0.05% Trypsin-EDTA and resuspended in DMEM medium that was primed in advance in a 37 °C, 5% CO₂ incubator. Cells were counted using an automatic cell counter and transported in LoBind protein tubes, 100,000 cells/tube. MSC #2 cells were treated with Y201 CFSE-EVs and unlabelled-EVs respectively at a 10× concentration for 1 hr, 2 hr, 4 hr, 6 hr, 8 hr and 10 hr.

To determine if EV uptake is RGD dependent Cells were treated with 200 μM Gly-Arg-Gly-Asp-Ser-Pro (GRGDSP—SEQ ID NO: 16) or Gly-Arg-Ala-Asp-Ser-Pro (GRADSP—SEQ ID NO: 17) peptides (Sigma-Aldrich, SCP0157, SCP0156) for 6 hrs and peptides were removed by pelleting the cells and resuspending them in fresh DMEM medium. CFSE-EVs and unlabelled-EVs at a 10× concentration were introduced to cells for 4 hr. Cells were pelleted down by centrifugation at 300 g for 5 mins at 4 °C and washed twice with ice-cold PBS to remove any EVs that had not been uptaken. Cells were resuspended in 200 μl flow buffer (1% FBS in PBS) and EV uptake by the MSC #2 cells was determined by flow cytometry using the LX375 CytoFlex. The gains were adjusted as follows: i) Forward Scatter at 20, ii) Side Scatter at 40 and iii) Fluorescein isothiocyanate who detects the CFSE fluorescence at 40.

### Antigen-Induced Arthritis (AIA) Model of Inflammatory Arthritis

AIA was induced in male C57BI/6 mice (7-8 weeks) as previously described (Kehoe et al. Arthritis Res Ther 16, R148 (2014)). Swelling was assessed by measuring the difference in diameter between the arthritic (right) and non-arthritic (left) knee joints (in mm) using a digital micrometer.

Treatments comprising 15 μL at 100 ug/mL EVs in suspension in PBS or PBS alone controls were injected intra-articularly 1 day post arthritis induction through the patellar ligament into the right knee joint. Joint diameters were measured at 1-, 2- and 3-days post-injection. Four independent experiments were performed to assess impact on joint swelling and histopathology.

Animals were sacrificed for histological analysis at day 3 post arthritis induction. Joints were fixed in 10% neutral buffered formal saline and decalcified in formic acid for 4 days at 4 °C before paraffin embedding. Sections (5 μm) were stained with haematoxylin and eosin and mounted. H&E sections were scored for hyperplasia of the synovial intima (0=normal to 3=severe), cellular exudate (0=normal to 3=severe) and synovial infiltrate (0=normal to 5=severe) by two independent observers blinded to experimental groups. Scores were summated, producing a mean arthritis index.

### Results

### Y201 Characterisation

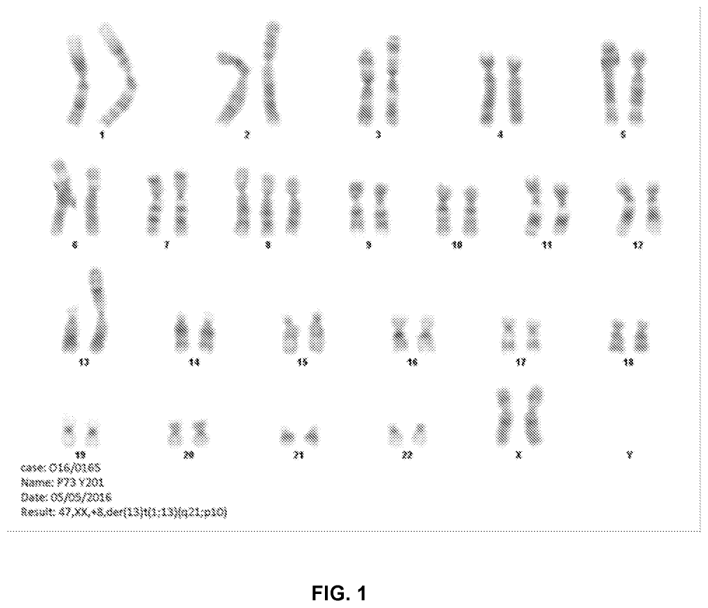

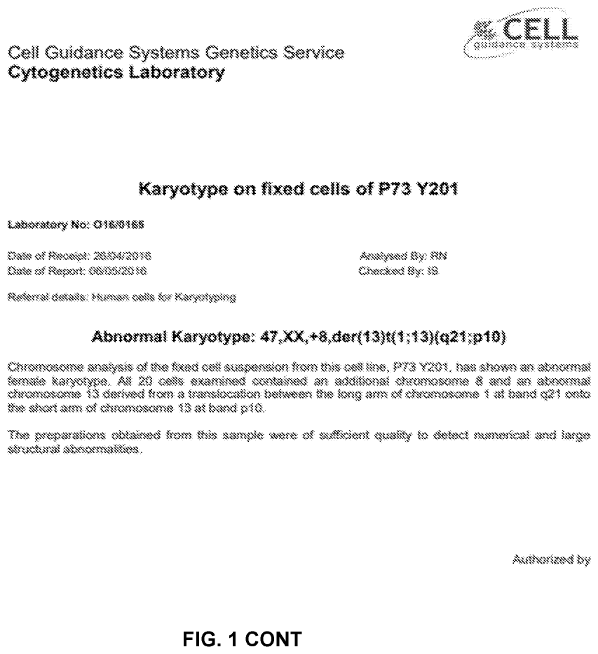

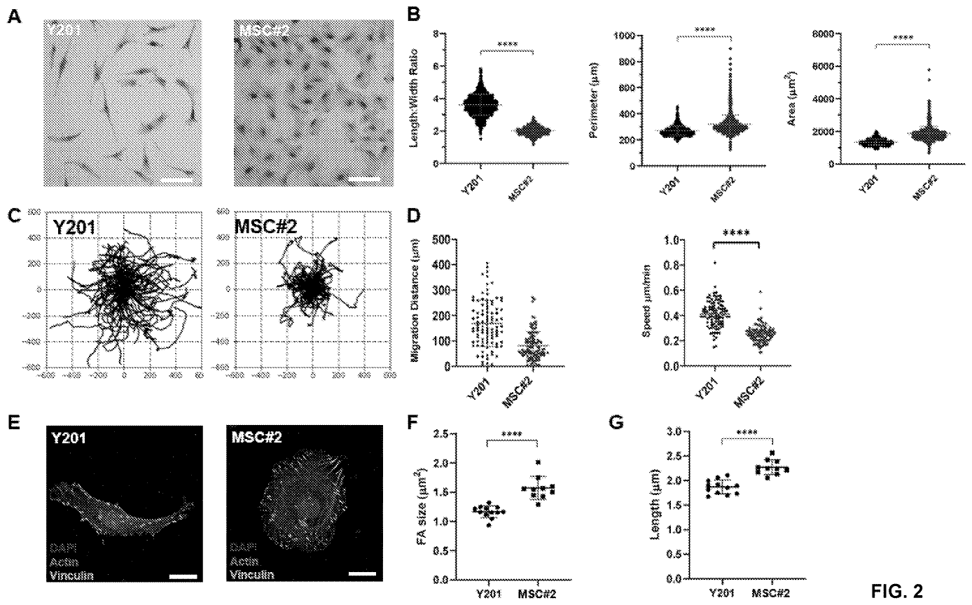

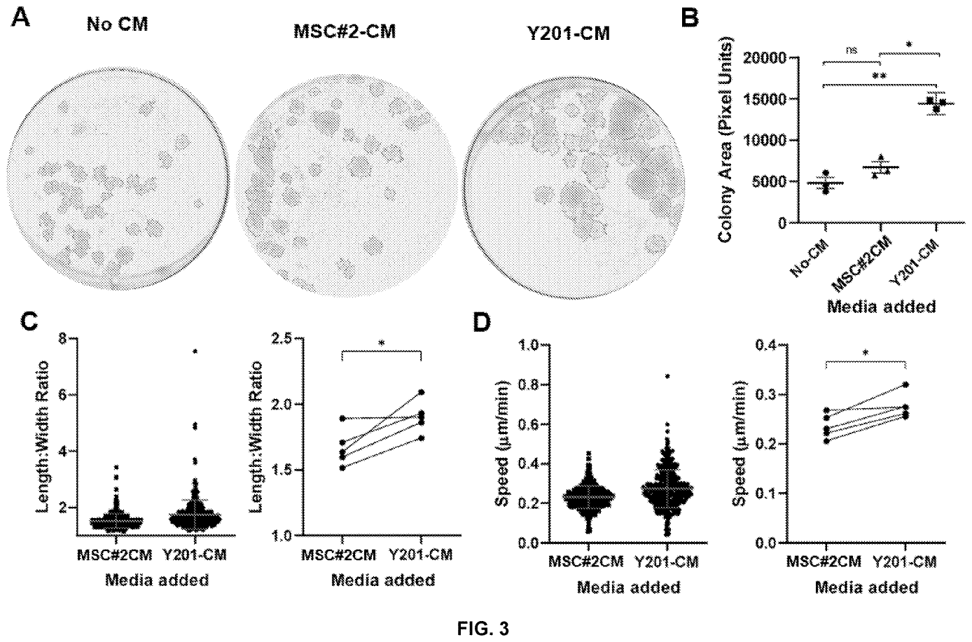

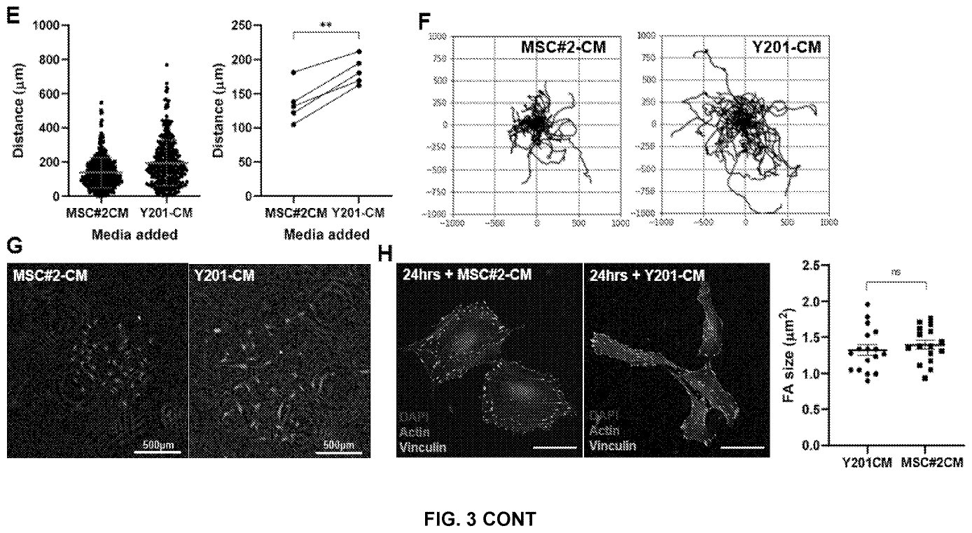

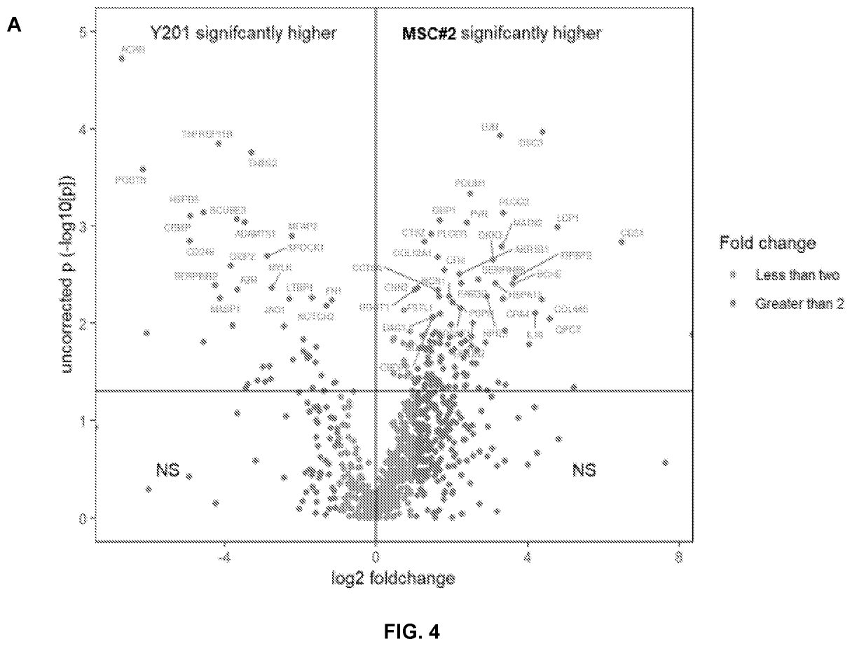

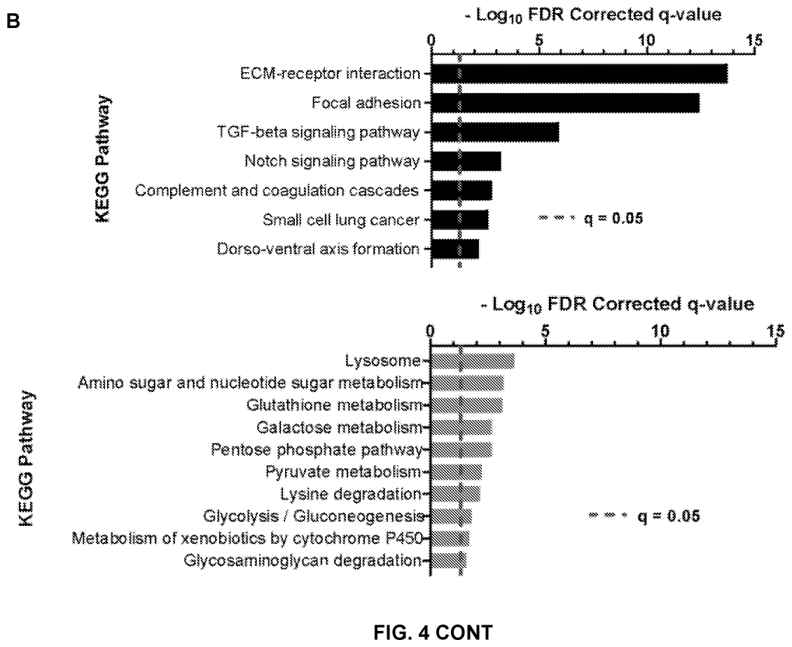

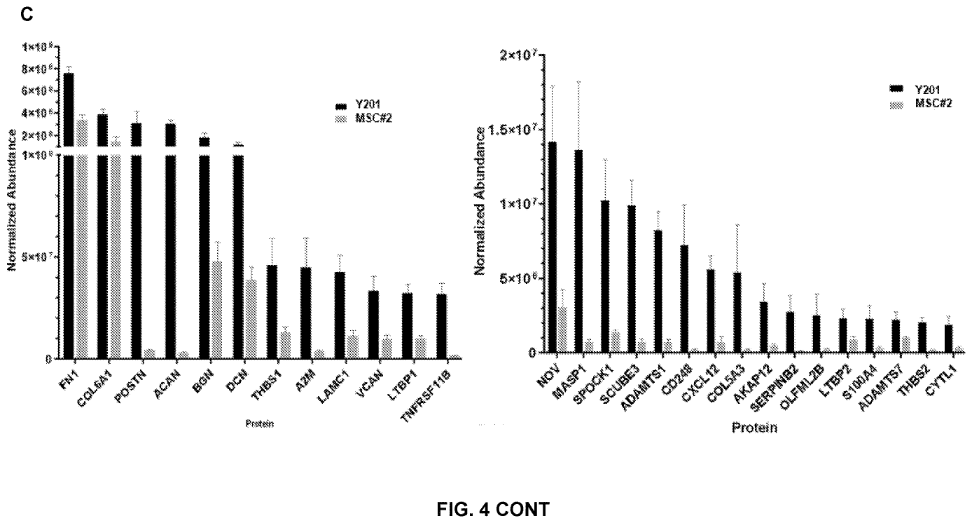

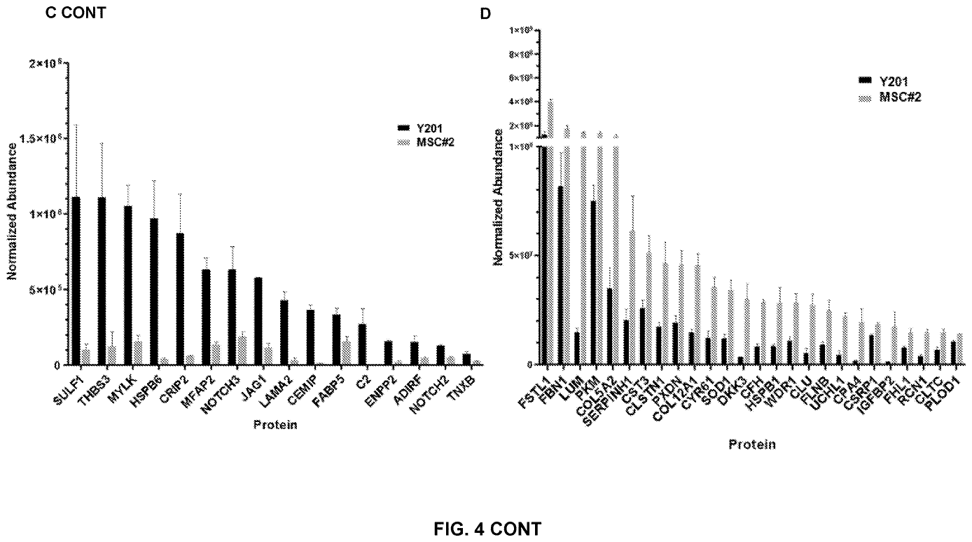

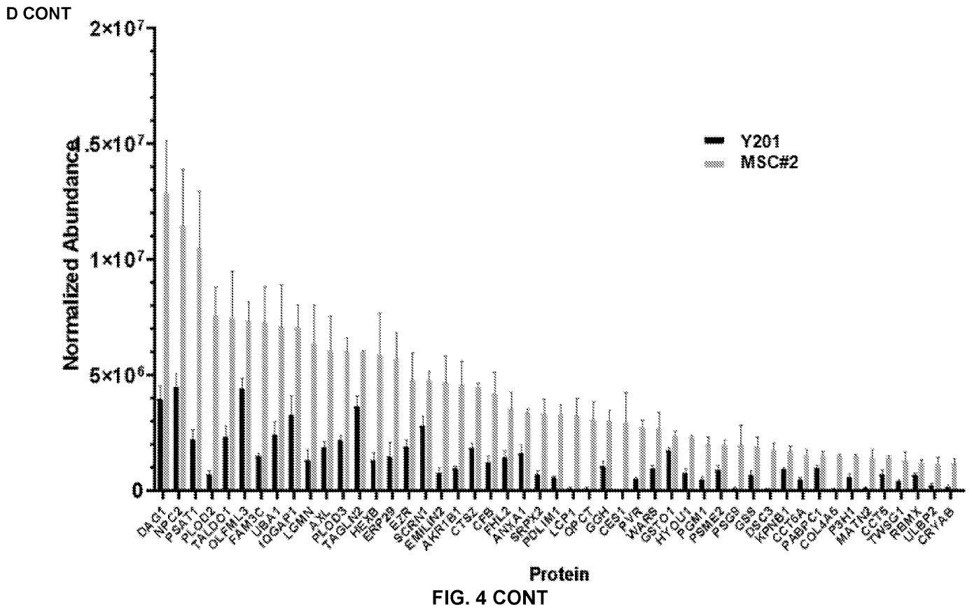

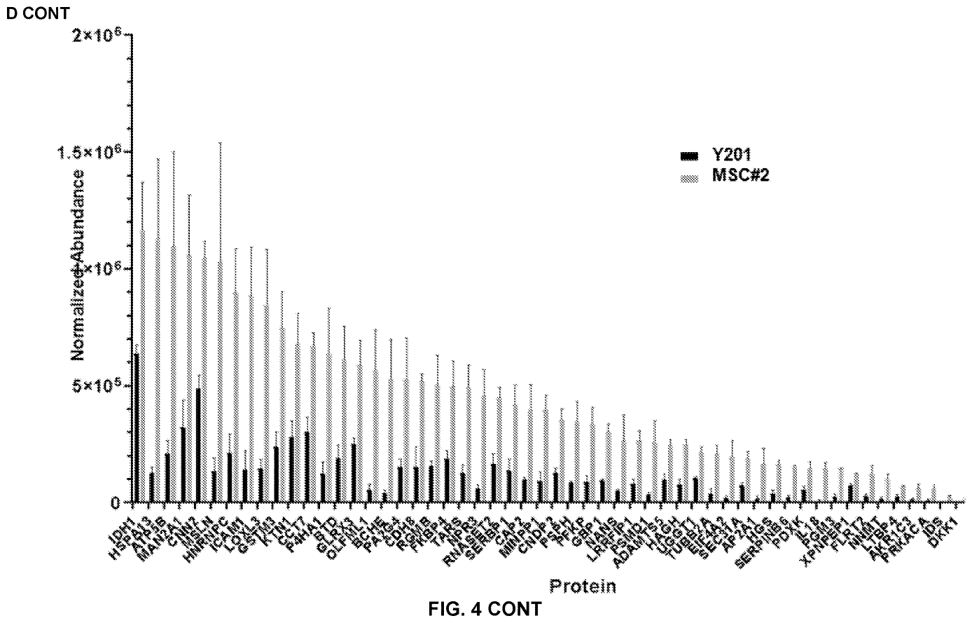

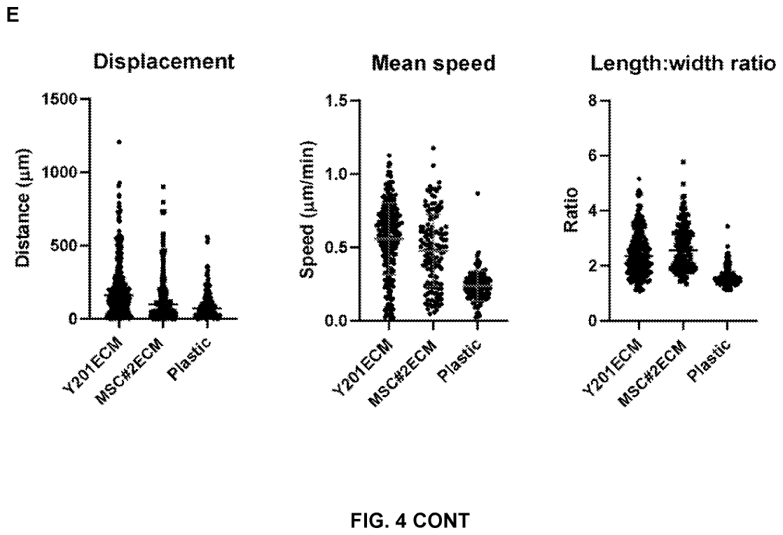

Y201 MSCs were generated by immortalisation with human telomerase reverse transcriptase (hTERT) and characterised as previously described (James et al, 2015, Kay et al, 2022). In addition, the inventors observed that Y201 MSCs have a distinctive karyotype. All 20 cells examined contained an additional chromosome 8 and a chromosomal translocation between the long arm of chromosome 1 at band q21 onto the short arm of chromosome 13 at band p10 (FIG. 1). Y201 cells are typically elongated and migratory (FIG. 2) and the inventors have shown that the whole secretome (including its constituent extracellular matrix (ECM) components) produced by Y201 MSCs can alter the morphology and migratory behaviour of the comparator MSC sub-line MSC #2 to take on the behavioural characteristics of Y201 MSCs (FIGS. 3 and 4). These findings demonstrate that the factors secreted by Y201 cells can induce functional changes in other cell types that may have therapeutic applications, for example increased migration and tissue regeneration. Overall, the secretome positively affects the comparator MSC line, the secretome induces functional changes in the comparator MSC line resulting in said cells becoming more akin to stem-like MSCs in terms of morphology and migratory behaviour. A large proportion of MSCs found in situ do not have stem-like properties therefore, administration of the secretome to a wound site may stimulate proliferation by reprogramming resident MSCs to exhibit stem-like behaviour.

### EV Characterisation

Size, Morphology, Protein and microRNA Cargo

EVs were initially isolated using differential ultracentrifugation, which separates different EV subpopulations based on their density. Serum-free medium was conditioned by Y201 cells and centrifuged at different speeds, 10,000 g and 100,000 g, to yield two fractions designated 10k and 100k, respectively.

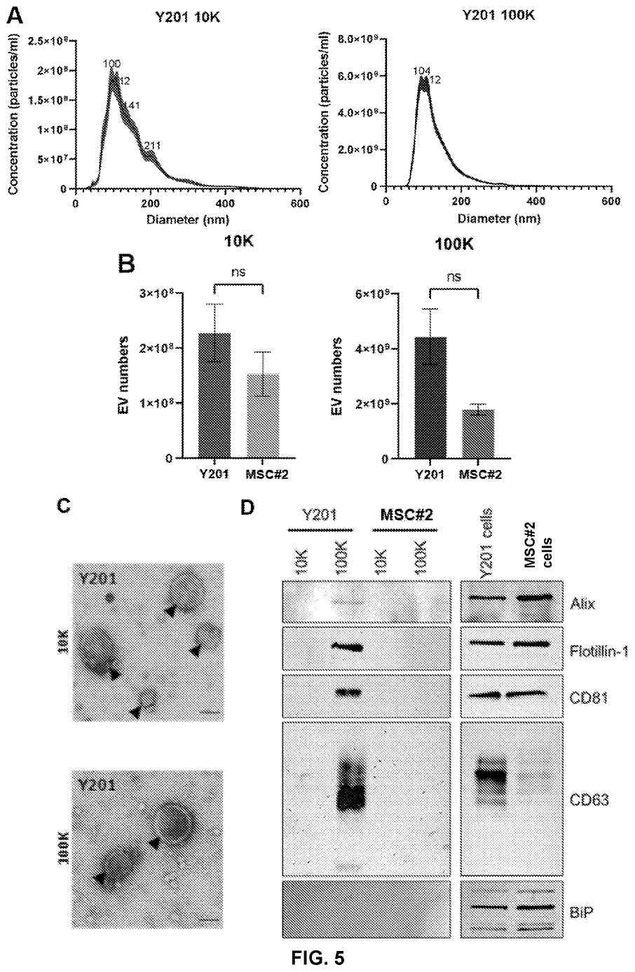

EVs were characterised using nanoparticle tracking analysis (NTA) for yield and size distribution, transmission electron microscopy (TEM) and image analysis for morphology and western blotting for EV-specific markers. The inventors observed peak EV sizes of 100 nm, 112 nm, 141 nm and 211 nm in the Y201 10K fraction. The Y201 100k fraction showed reproducible peak average diameters of 104 nm and 112 nm with typical morphologies by TEM. EV yields were approximately 2.5×10⁸ and 4.5×10⁹ EVs per million cells for the Y201 10k and 100k fractions respectively. Y201 EVs were positive for specific markers Alix, Flotillin-1, CD81 and CD63 in the 100k fraction and negative for the endosomal marker BiP (FIG. 5). EV markers were not detected in the 10k fractions, which were also more variable in terms of size based on the NTA data, therefore future work focussed on the 100k EV fractions.

The inventors performed full proteomic and microRNA screening of EV cargo from the Y201 100k EV fraction with follow-up bioinformatics analysis (Tables 2, 3 and 4).

TABLE 2
Top 200 identified proteins in Y201 conditioned medium.
Conditioned media was analysed using LC-MS and searched against
the human subset of UniProt database. Abundance was quantified
using relative peak areas of non-conflicting peptides and Progenesis QI.
Secreted

| | | | |
|---|---|---|---|
| SPARC | NUCB1 | PEBP1 | PDIA6 |
| COL1A2 | TNFRSF11B | LOX | TPM4 |
| FN1 | AHNAK | WDR1 | SERPINE1 |
| COL1A1 | PDIA3 | ARHGDIA | PDLIM5 |
| IGFBP7 | HSPA8 | LOXL2 | PSAP |
| COL6A1 | CALD1 | PLOD1 | CRIM1 |
| VIM | CST3 | SPOCK1 | LASP1 |
| POSTN | GAPDH | NID1 | VCP |
| ACAN | STC2 | SCUBE3 | ACLY |
| TIMP1 | PPIB | ITGBL1 | YWHAB |
| BGN | IGFBP3 | GDI2 | MARCKS |
| FLNA | COL5A1 | ALCAM | TFPI |
| ENO1 | TPI1 | CLEC3B | NPC2 |
| FBLN1 | MFAP5 | PTX3 | OLFML3 |
| FSTL1 | SERPINH1 | FLNB | GSN |
| DCN | ACTN4 | CCDC80 | UCHL1 |
| ECM1 | PGK1 | HSPB1 | HSPA4 |
| COL6A2 | SERPINE2 | MDH1 | PRDX6 |
| PFN1 | PXDN | PEA15 | GALNT2 |
| ALDOA | TXN | CFH | VASN |
| ACTB | LGALS3BP | ADAMTS1 | HNRNPA1 |
| FBN1 | CLSTN1 | TLN1 | DAG1 |
| C1R | MDH2 | PGAM1 | ITM2B |
| COL6A3 | ITIH2 | ZYX | CSPG4 |
| PKM | MFGE8 | PLS3 | RCN1 |
| TGFBI | PLEC | CTGF | HSPA1A |
| COL4A2 | LUM | ABI3BP | RNH1 |
| B2M | COL12A1 | YWHAE | ADAM10 |
| LGALS1 | DPYSL2 | FHL1 | CD109 |
| TIMP2 | CLIC4 | RELN | LAMA4 |
| PCOLCE | NOV | PARK7 | TAGLN2 |
| IGFBP6 | GSTP1 | CDH2 | CTSL |
| LDHA | LMNA | CD248 | GANAB |
| COL3A1 | VCL | BMP1 | DKK3 |
| QSOX1 | MASP1 | ANXA2 | TKT |
| IGFBP4 | FLNC | ADAM9 | VAT1 |
| HSPG2 | HTRA1 | TMSB10 | AKAP12 |
| COL4A1 | CSRP1 | BASP1 | INHBA |
| PPIA | LAMB1 | CLTC | SYNCRIP |
| CTSB | S100A11 | CALU | SERPING1 |
| THBS1 | TAGLN | MYH9 | IQGAP1 |
| C1S | MSN | LDHB | SEMA7A |
| A2M | CYR61 | SRGN | GBE1 |
| EFEMP1 | C4A | MAP4 | TXNRD1 |
| LAMC1 | CLIC1 | TNC | IGFBP1 |
| MMP2 | SOD1 | CXCL12 | SPTAN1 |
| COL5A2 | PRDX1 | COL5A3 | GPI |
| VCAN | ACTN1 | ALDOC | TXNDC5 |
| CFL1 | HSPA5 | CLU | MYL6 |
| LTBP1 | EEF2 | CFL2 | MAP1B |

TABLE 3
Top 200 identified proteins in Y201 EVs. Isolated EVs were
analysed using LC-MS and searched against the human subset of
UniProt database. Abundance was quantified using relative peak
areas of non-conflicting peptides and Progenesis QI.
EV only

| | | | |
|---|---|---|---|
| FN1 | FBLN1 | ITGAV | VDAC2 |
| MFGE8 | TUBA4A | BGN | VAT1 |
| COL6A3 | LRP1 | UBB | HSPB1 |
| THBS1 | ITGA3 | PPIB | YWHAQ |
| ACTB | HSP90B1 | STOM | ACTN4 |
| HSPG2 | AHNAK | CKAP4 | GNG12 |
| TSPAN14 | LAMB1 | ATP5A1 | ATP2B4 |
| ITIH2 | CFL1 | RPL4 | THBS2 |
| RELN | COL3A1 | ENO1 | SCUBE3 |
| COL6A1 | MVP | TAGLN | DSTN |
| TLN1 | KRT2 | HBA1 | VASP |
| EDIL3 | GNAI2 | CCT5 | C5 |
| KRT1 | VTN | BASP1 | SERPINE1 |
| FLNA | P4HB | CALU | PRSS23 |
| COL1A1 | VCL | ATP1A1 | YWHAE |
| COL6A2 | ANXA1 | S100A11 | CTNND1 |
| PTX3 | AGRN | CLIC4 | SERPINE2 |
| TNXB | C4A | CALM1 | SERPIND1 |
| MYH9 | TGFBI | GNB2 | PDIA6 |
| ITGB3 | LGALS1 | JCHAIN | C1QTNF3 |
| COL1A2 | PDIA3 | POSTN | GSN |
| DMBT1 | MYL6 | CEMIP | CTGF |
| SRI | ALDOA | LAMA4 | VDAC1 |
| ANXA2 | PFN1 | HSP90AA1 | RAB14 |
| GAPDH | EEF2 | NID1 | CYB5R3 |
| COL12A1 | ITGA5 | PGD | ACTA2 |
| PLSCR3 | DYNC1H1 | TUBB4B | PGAM1 |
| EEF1A1P5 | PCOLCE | RPSA | COL21A1 |
| KRT9 | PXDN | RAC1 | CANX |
| LGALS3BP | APOB | ENG | NPTN |
| PKM | MYOF | LDHA | RPS2 |
| VIM | LIMS1 | MYO1C | PPIA |
| TUBB1 | CD44 | MDH2 | FERMT2 |
| TNC | CSPG4 | LAMB2 | HGFAC |
| CD81 | SVEP1 | IGSF8 | MARCKS |
| NID2 | SLC3A2 | EMILIN1 | PLOD2 |
| CLTC | VCAN | B2M | HIST2H2AC |
| KRT10 | RPS4X | MAMDC2 | PDCD6 |
| ACAN | CD9 | PDCD6IP | ANXA4 |
| ANXA5 | NAV1 | APOM | MYH4 |
| ITGB1 | CALR | ITGA7 | SLC25A6 |
| ANXA6 | ANPEP | CD151 | TFRC |
| THY1 | ALCAM | EHD1 | CAV1 |
| SDCBP | RAB7A | RPLP2 | RPL10 |
| NT5E | PTGFRN | HSPD1 | FSTL1 |
| HSPA8 | SERPINH1 | GANAB | PLOD1 |
| HTRA1 | LAMA5 | SLC1A5 | RPLP0P6 |
| CD59 | PSMD2 | EHD2 | BMP1 |
| LOXL2 | ITGA6 | ANXA11 | PGK1 |
| LAMC1 | RTN4 | HIST1H2BD | YWHAH |

TABLE 4
Identified miRNAs in Y201 EVs. Y201 EVs were analysed for
their miRNA content by NanoString array. Abundance was quantified
relative to baseline readings (undetected probes).

| Probe Name | Accession # | Probe Name | Accession # |
|---|---|---|---|
| hsa-miR-4454 + | MIMAT0018976 | hsa-miR-378h | MIMAT0018984 |
| hsa-miR-7975 | | | |
| hsa-miR-451a | MIMAT0001631 | hsa-miR-548q | MIMAT0011163 |
| hsa-miR-125b-5p | MIMAT0000423 | hsa-miR-520h | MIMAT0002867 |
| hsa-miR-144-3p | MIMAT0000436 | hsa-miR-30e-5p | MIMAT0000692 |
| hsa-miR-142-3p | MIMAT0000434 | osa-miR414 | MIMAT0001330 |
| hsa-miR-23a-3p | MIMAT0000078 | hsa-let-7e-5p | MIMAT0000066 |
| hsa-let-7b-5p | MIMAT0000063 | hsa-miR-99a-5p | MIMAT0000097 |
| hsa-miR-21-5p | MIMAT0000076 | hsa-miR-382-5p | MIMAT0000737 |
| hsa-miR-4286 | MIMAT0016916 | hsa-miR-337-3p | MIMAT0000754 |
| hsa-miR-199a-3p + | MIMAT0000232 | hsa-miR-4536-5p | MIMAT0019078 |
| hsa-miR-199b-3p | | | |
| hsa-miR-29a-3p | MIMAT0000086 | hsa-miR-20a-5p + | MIMAT0000075 |
| | | hsa-miR-20b-5p | |
| hsa-miR-100-5p | MIMAT0000098 | hsa-miR-549a | MIMAT0003333 |
| hsa-miR-630 | MIMAT0003299 | hsa-miR-196a-5p | MIMAT0000226 |
| hsa-let-7a-5p | MIMAT0000062 | hsa-miR-155-5p | MIMAT0000646 |
| hsa-miR-29b-3p | MIMAT0000100 | hsa-miR-26a-5p | MIMAT0000082 |
| hsa-miR-221-3p | MIMAT0000278 | hsa-miR-361-5p | MIMAT0000703 |
| hsa-miR-145-5p | MIMAT0000437 | hsa-miR-1255b-5p | MIMAT0005945 |
| hsa-miR-579-3p | MIMAT0003244 | hsa-miR-19a-3p | MIMAT0000073 |
| hsa-miR-16-5p | MIMAT0000069 | hsa-miR-548d-3p | MIMAT0003323 |
| hsa-miR-15b-5p | MIMAT0000417 | hsa-miR-183-5p | MIMAT0000261 |
| hsa-miR-126-3p | MIMAT0000445 | hsa-miR-514b-5p | MIMAT0015087 |
| hsa-let-7i-5p | MIMAT0000415 | hsa-miR-130a-3p | MIMAT0000425 |
| hsa-miR-376a-3p | MIMAT0000729 | hsa-let-7c-5p | MIMAT0000064 |
| hsa-miR-93-5p | MIMAT0000093 | hsa-miR-186-5p | MIMAT0000456 |
| hsa-miR-320e | MIMAT0015072 | hsa-miR-548y | MIMAT0018354 |
| hsa-miR-612 | MIMAT0003280 | ath-miR159a | MIMAT0000177 |
| hsa-miR-223-3p | MIMAT0000280 | hsa-miR-548g-3p | MIMAT0005912 |
| hsa-miR-1253 | MIMAT0005904 | hsa-miR-122-5p | MIMAT0000421 |
| hsa-miR-191-5p | MIMAT0000440 | hsa-miR-30b-5p | MIMAT0000420 |
| hsa-miR-575 | MIMAT0003240 | osa-miR442 | MIMAT0001605 |
| hsa-miR-499a-5p | MIMAT0002870 | hsa-miR-1827 | MIMAT0006767 |
| hsa-miR-19b-3p | MIMAT0000074 | hsa-miR-423-5p | MIMAT0004748 |
| hsa-miR-24-3p | MIMAT0000080 | hsa-miR-3202 | MIMAT0015089 |
| hsa-miR-150-5p | MIMAT0000451 | hsa-miR-381-3p | MIMAT0000736 |
| hsa-miR-25-3p | MIMAT0000081 | hsa-miR-553 | MIMAT0003216 |
| hsa-miR-15a-5p | MIMAT0000068 | hsa-miR-613 | MIMAT0003281 |
| hsa-miR-644a | MIMAT0003314 | hsa-miR-1915-3p | MIMAT0007892 |
| hsa-miR-1260a | MIMAT0005911 | hsa-miR-34a-5p | MIMAT0000255 |
| hsa-miR-365a-3p + | MIMAT0000710 | hsa-miR-873-3p | MIMAT0022717 |
| hsa-miR-365b-3p | | | |
| hsa-miR-30c-5p | MIMAT0000244 | hsa-miR-548n | MIMAT0005916 |
| hsa-miR-376c-3p | MIMAT0000720 | hsa-miR-1973 | MIMAT0009448 |
| hsa-miR-136-5p | MIMAT0000448 | hsa-miR-212-3p | MIMAT0000269 |
| hsa-miR-140-5p | MIMAT0000431 | hsa-miR-1234-3p | MIMAT0005589 |
| hsa-miR-199a-5p | MIMAT0000231 | hsa-miR-185-5p | MIMAT0000455 |
| hsa-let-7d-5p | MIMAT0000065 | hsa-miR-548j-3p | MIMAT0026737 |
| hsa-miR-379-5p | MIMAT0000733 | hsa-miR-411-5p | MIMAT0003329 |
| hsa-miR-27b-3p | MIMAT0000419 | hsa-miR-579-5p | MIMAT0026616 |
| hsa-miR-3144-3p | MIMAT0015015 | hsa-miR-574-5p | MIMAT0004795 |
| hsa-miR-127-3p | MIMAT0000446 | hsa-miR-26b-5p | MIMAT0000083 |
| hsa-miR-22-3p | MIMAT0000077 | hsa-miR-513c-3p | MIMAT0022728 |
| hsa-let-7g-5p | MIMAT0000414 | hsa-miR-1268a | MIMAT0005922 |
| hsa-miR-129-5p | MIMAT0000242 | hsa-miR-30d-5p | MIMAT0000245 |
| hsa-miR-543 | MIMAT0004954 | hsa-miR-450a-5p | MIMAT0001545 |
| hsa-miR-106b-5p | MIMAT0000680 | hsa-miR-337-5p | MIMAT0004695 |
| hsa-miR-99b-5p | MIMAT0000689 | hsa-miR-608 | MIMAT0003276 |
| hsa-miR-4531 | MIMAT0019070 | hsa-miR-125a-5p | MIMAT0000443 |
| hsa-miR-374a-5p | MIMAT0000727 | hsa-miR-301a-3p | MIMAT0000688 |
| hsa-miR-1257 | MIMAT0005908 | hsa-miR-193a-5p + | MIMAT0004614 |
| | | hsa-miR-193b-5p | |
| hsa-miR-377-3p | MIMAT0000730 | hsa-miR-331-3p | MIMAT0000760 |
| hsa-miR-216b-5p | MIMAT0004959 | hsa-miR-3136-5p | MIMAT0015003 |
| hsa-miR-1290 | MIMAT0005880 | hsa-miR-193a-3p | MIMAT0000459 |
| hsa-miR-23b-3p | MIMAT0000418 | hsa-miR-214-3p | MIMAT0000271 |
| hsa-miR-181a-5p | MIMAT0000256 | hsa-miR-378i | MIMAT0019074 |
| hsa-miR-495-3p | MIMAT0002817 | hsa-miR-556-5p | MIMAT0003220 |
| hsa-miR-422a | MIMAT0001339 | hsa-miR-888-5p | MIMAT0004916 |
| hsa-miR-98-5p | MIMAT0000096 | hsa-miR-6721-5p | MIMAT0025852 |
| hsa-miR-1285-5p | MIMAT0022719 | hsa-miR-4443 | MIMAT0018961 |
| hsa-miR-222-3p | MIMAT0000279 | hsa-miR-128-1-5p | MIMAT0026477 |
| hsa-miR-1255a | MIMAT0005906 | hsa-miR-424-5p | MIMAT0001341 |
| hsa-miR-548ah-5p | MIMAT0018972 | hsa-miR-509-5p | MIMAT0004779 |
| hsa-miR-148a-3p | MIMAT0000243 | | |

### Analysis of Y201 EV Protein Cargo

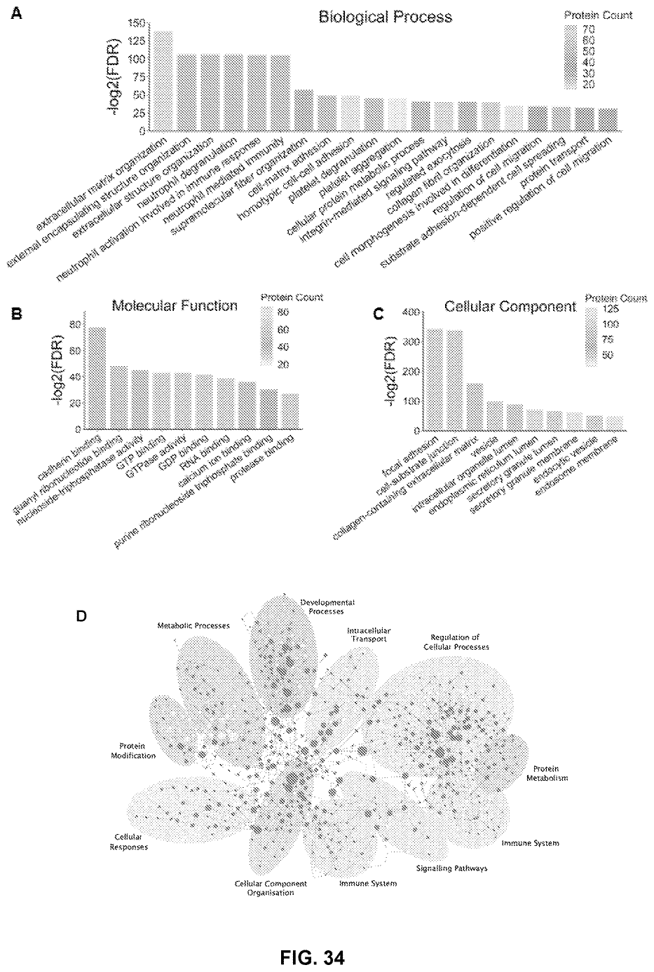

To provide further insight into the nature of the enhanced Y201 EVome, GO analysis against Biological Process, Molecular Function, and Cellular Component genesets was carried out. In terms of Biological Process, the Y201 EVome is predominantly enriched in organisation and structure of the extracellular matrix, immunomodulatory activity mediated through neutrophils, and various adhesion and migration processes (FIG. 34A). The Molecular Functions of these proteins largely pertain to the binding of cadherin and various nucleic acids including GDP, nucleic triphosphates, and RNAs (FIG. 34B). Enrichment against the Cellular Components geneset revealed that these proteins are significantly present in focal adhesions and cell-substrate junctions, to a relatively substantial degree. (FIG. 34C). To gain a broader perspective of potential Y201 EV functions in vivo, the Y201 EVome was assessed for enrichment against the GO Biological Process geneset in the BINGO plugin for Cytoscape, which formulates clustered networks of significantly enriched processes (FIG. 34D). Sizeable clusters formed around similar significant processes relating to the immune system, regulation of cellular processes, developmental processes, and organisation of cellular components.

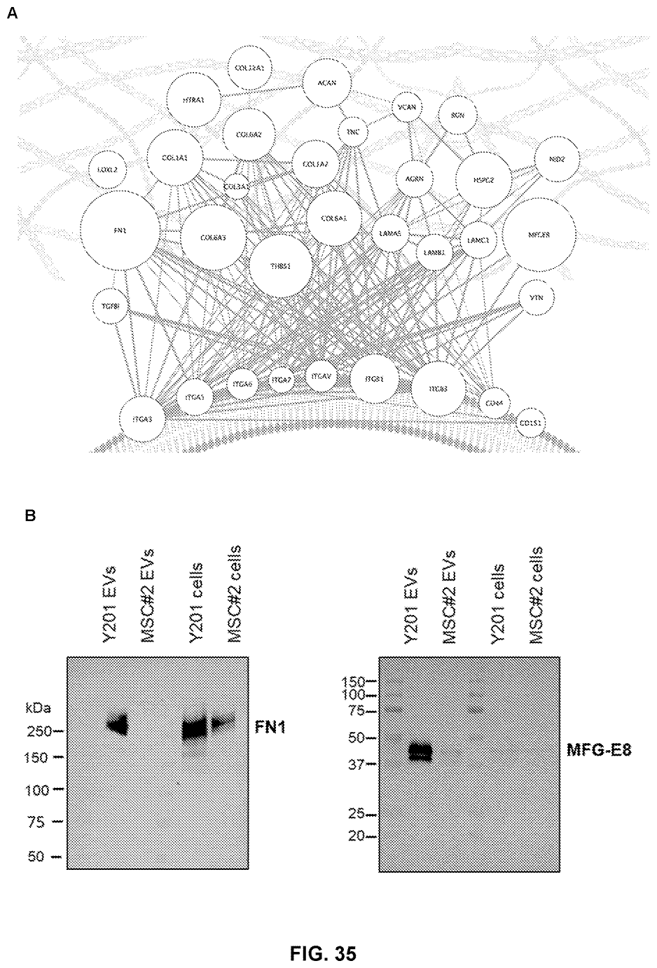

Considering the most abundant Y201 EV proteins were associated with ECM organisation, the inventors attempted to model the extravesicular corona by constructing a protein-protein interaction (PPI) network in STRING (FIG. 35A). The inventors identified a large number of PPIs between ECM component and cognate vesicle membrane integrins and tetraspanins. Fibronectin (FN1) and milk fat globule epidermal growth factor 8 (MFGE8) were amongst the most abundant presumptive Y201 EV coronal proteins, which the inventors confirmed by western blot analysis relative to MSC #2 EVs (FIG. 35B).

### EV Bioactivity

### Cargo Delivery

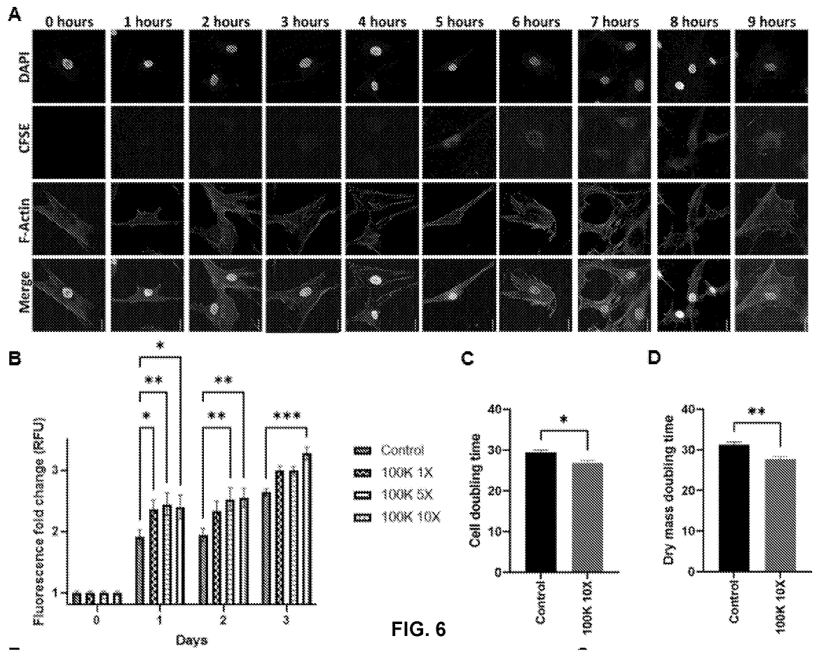

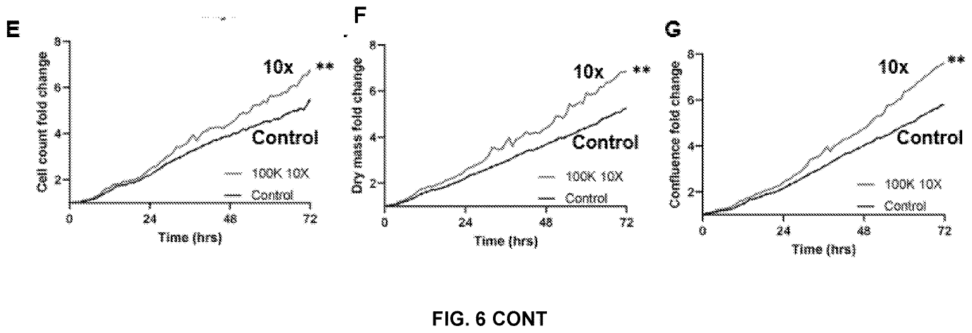

Using EVs labelled with a membrane-specific fluorescent dye (CFSE) and fluorescence microscopy, the inventors showed that Y201 EVs are taken up by primary bone marrow MSCs, with intracellular EVs first appearing within 5 hours of treatment, demonstrating potential for EV-mediated cargo delivery (FIG. 6A). The inventors have also shown that Y201 EVs can be loaded with the anti-cancer drug doxorubicin and delivered to osteosarcoma cell lines (U2OS) to inhibit proliferation (data not shown).

### Uptake of EVs by Different MSC Subtypes

MSC #2 cells were treated with Y201 CFSE-labelled EVs and flow cytometry was used to quantify EV uptake. A gating strategy using the Cytoflex LX375 was set up using the following parameters. An FSC against SSC plot was created to discriminate between cells and debris in suspension. After gating out the debris, single cells were distinguished by plotting FSC-H against FSC-A. Cells that were not treated with stained EVs were used to identify the autofluorescence of the cells by plotting SSC against the CFSE signal. Finally, cells treated with CFSE-EVs were used to measure the successful detection of EV uptake.

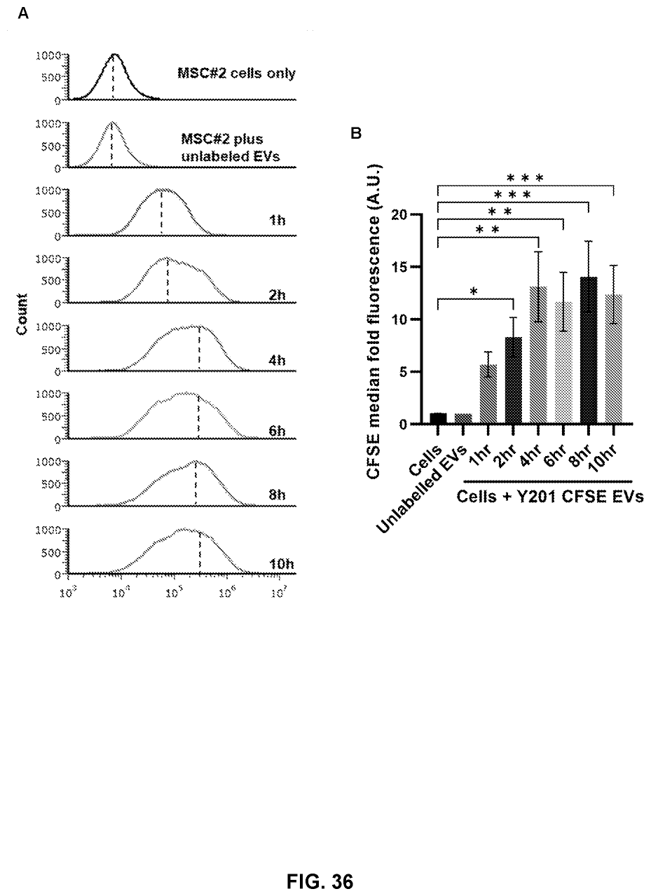

The time course of EV uptake was determined by flow cytometry. Cells were treated with unlabelled or CFSE-labelled EVs for up to 10 hrs followed by flow cytometry on living cells. MSC #2 cells exposed to CFSE-Y201 EVs increased in fluorescence over the first 4 hrs and levels remained elevated for up to 10 hrs (FIGS. 36A&B).

### RGD-Integrin Mediated Uptake and Function of Y201 EVs

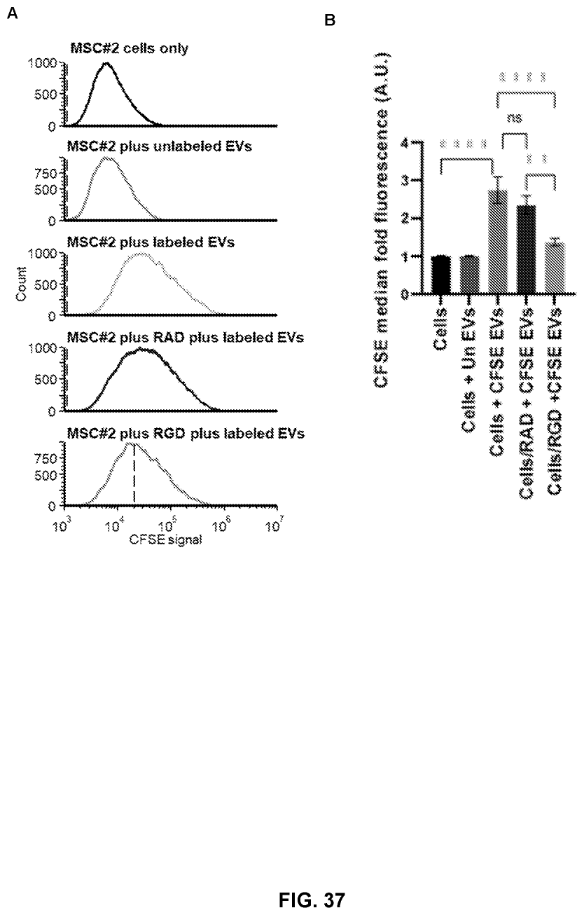

Considering the evidence for an enriched ECM-based corona in Y201 EVs, particularly the abundance of RGD-containing proteins (FN1 and MFGE8), without wishing to be bound by theory, the inventors believed that these EV subtypes were preferentially taken up by integrin-mediated endocytosis. MSC #2 cells were used as model target cells and treated with GRGDSP (SEQ ID NO: 16) integrin blocking peptides or GRADSP (SEQ ID NO: 17) peptide controls for 6 hrs followed by exposure to CFSE stained Y201 EVs. Using flow cytometry, the inventors demonstrated that Y201 EV uptake was significantly inhibited by RGD blockade (GRGDSP (SEQ ID NO: 16)) compared to controls (FIG. 37).

### Regenerative Effects of Y201 EVs

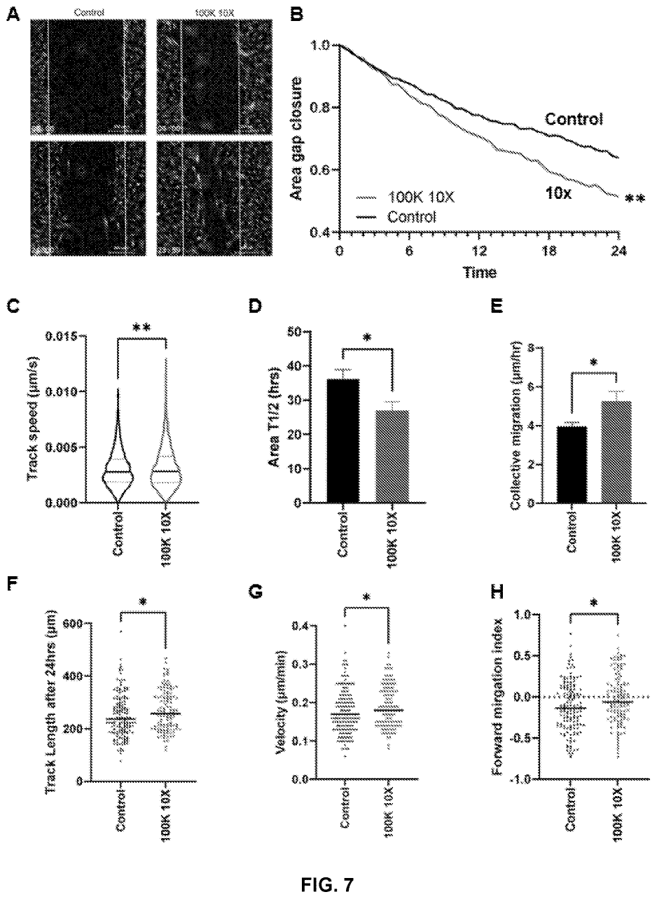

EV functionality was tested initially using Y201 EVs on another MSC sub-line (MSC #2). Dosages were determined by proportional EV yield per cell and multiplied by a factor of 1 (1×), 5 (5×), and 10 (10×), such that each cell was exposed to EVs from 1-fold, 5-fold, or 10-fold more cells. Y201 EVs significantly increased MSC #2 proliferation (FIG. 6B-G). Additionally, MSC #2 migration was enhanced by Y201 EVs (FIG. 7). This supports a regenerative therapeutic application for Y201 EVs through the stimulation of cell proliferation and migration.

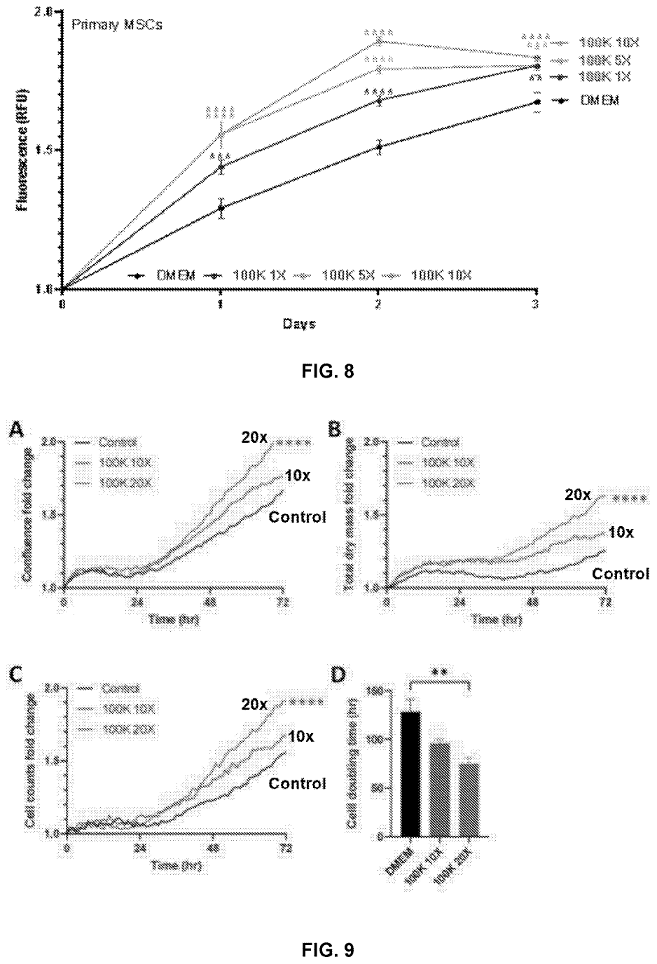

The inventors translated these findings into primary cells by treating human bone marrow MSCs from osteoarthritis patients, derived from waste bone following a total joint replacement, with Y201 EVs. The inventors observed a dose-dependent increase in proliferation compared to untreated controls, confirming that Y201 EVs can enhance cell proliferation, even in primary cells from aged donors (FIG. 8).

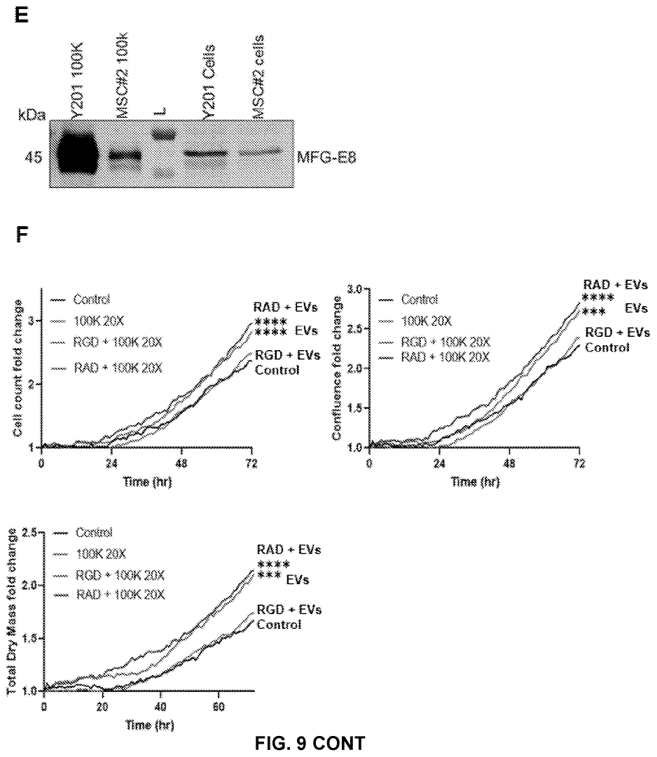

In disorders such as juvenile idiopathic arthritis, as well as others including rheumatoid arthritis or osteoarthritis, there is a gradual destruction of cartilage tissue in the joint. Therefore, the inventors determined the effects of Y201 EVs on articular chondrocyte proliferation. Chondrocytes are the only specialised cell type found in the cartilage tissue and are responsible for functional and structural integrity of cartilage. Articular chondrocytes were taken from the surface of waste joint tissue following total joint replacement surgery. Results showed that a significant dose-dependent increase in chondrocyte proliferation as well as a decrease in chondrocyte doubling time was achieved with Y201 EV treatment (FIG. 9).

Proteomic EV analysis revealed that the MFG-E8 protein is highly enriched in Y201 EVs versus MSC #2 EVs and that was further confirmed by western blotting (FIG. 9). MFG-E8 is able to bind EVs, enriched in phosphotidylserine, and interact with target cells via alphavbeta3/alphavbeta5 integrins and the RGD binding motif. The inventors hypothesise that Y201 derived EVs can be taken up by cells via integrin-mediated endocytosis. AC cells were treated with an RGD-containing blocking peptide, GRGDSP (SEQ ID NO. 16), or a control peptide, GRADSP (SEQ ID NO. 17) and the effect of Y201 EVs on their proliferation rate was monitored over 72 hrs. The inventors confirmed that Y201 EVs increase AC proliferation compared to untreated controls and that this effect was blocked by exposure to GRGDSP (SEQ ID NO. 16) but not GRADSP (SEQ ID NO. 17) peptides (FIG. 9).

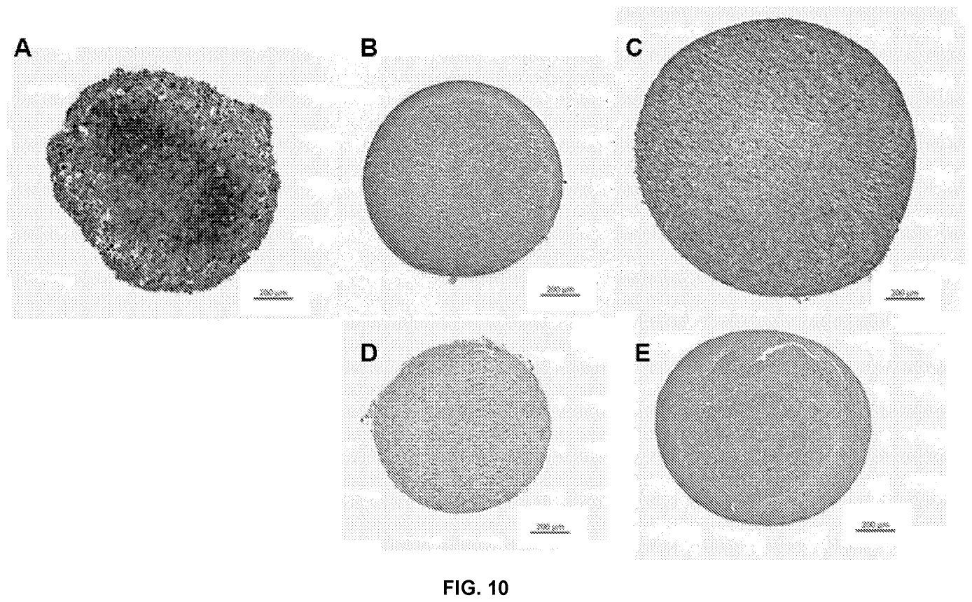

Using bone marrow MSCs from an osteoarthritis patient donor, the inventors investigated the effect of Y201 EVs on chondrogenic differentiation. The inventors showed that treatment with Y201 EVs enhances chondrogenic differentiation of MSCs, as visualised by Safranin O staining (FIG. 10). This demonstrates that Y201 EVs have tissue regenerative capabilities.

### Immune-Suppressive Effects of Y201 EVs

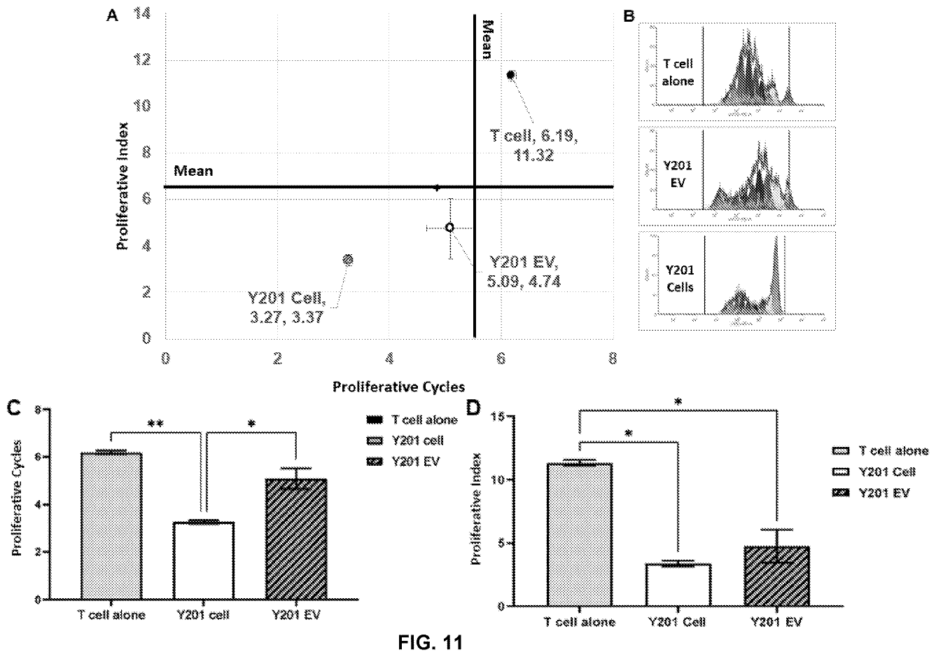

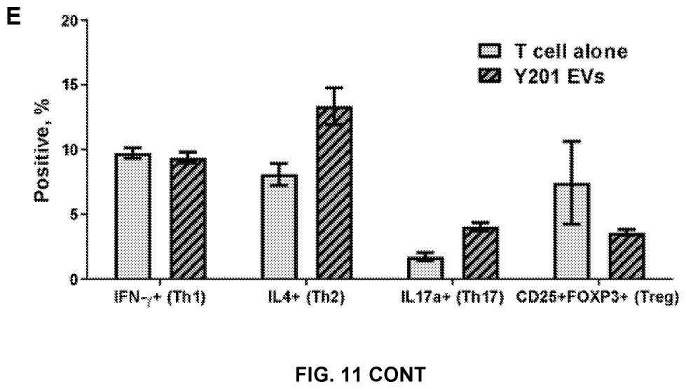

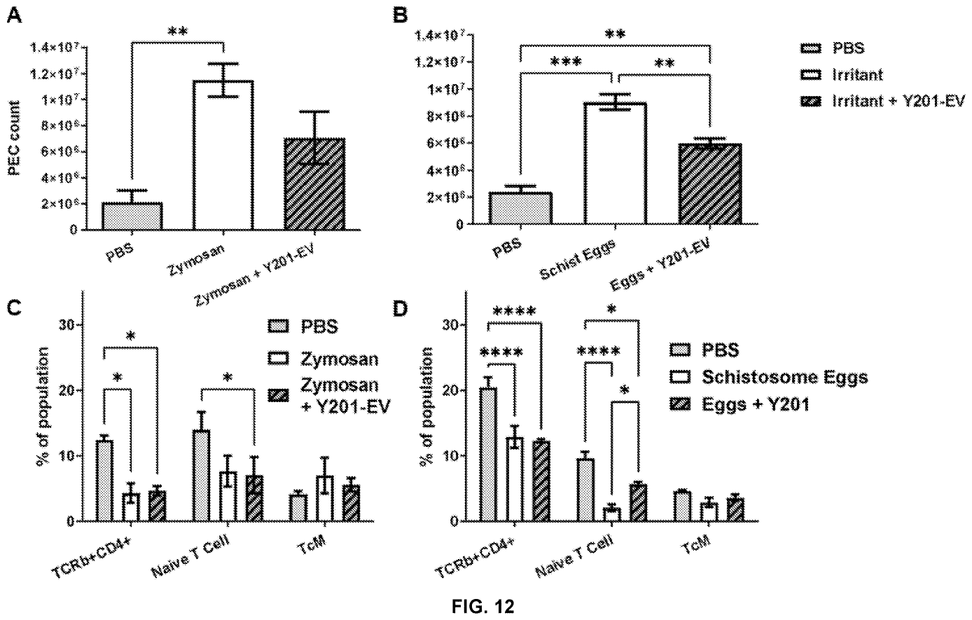

The inventors further studied the effect of Y201 EVs in an in vitro inflammation model. The inventors showed that EVs from Y201 cells moderate adaptive immune responses-such as those implicated in inflammatory diseases-via deactivation of CD4+ effector T cells in vitro, resulting in reduced proportions of proliferating immune cells (index) and reduced immune cell proliferative capacity (cycles). Further to this, Y201 EVs have the potential to counteract pro-inflammatory IFN-γ (Th1) responses through increased IL4 (Th2) differentiation (FIG. 11). The inventors progressed this work into an in vivo inflammation model, mimicking an infection-type immune response. Infectious agents may also play a role in initiation or perpetuation of the inflammatory process in autoimmune disease. Y201 EVs moderated pathogen responses to peritoneal infection in vivo through suppressed recruitment of immune cells and decreased T cell differentiation (FIG. 12).

The Y201 was modified using CRISPR/Cas9 to target a deletion of the FGFR3 gene and determine effects on cell behaviour. Data on the characteristics of the Y201 FGFR3 KO cell line are described below.

### In Vivo Assessment of Potential Therapeutic Efficacy of Y201 EVs in a Murine Arthritis Model

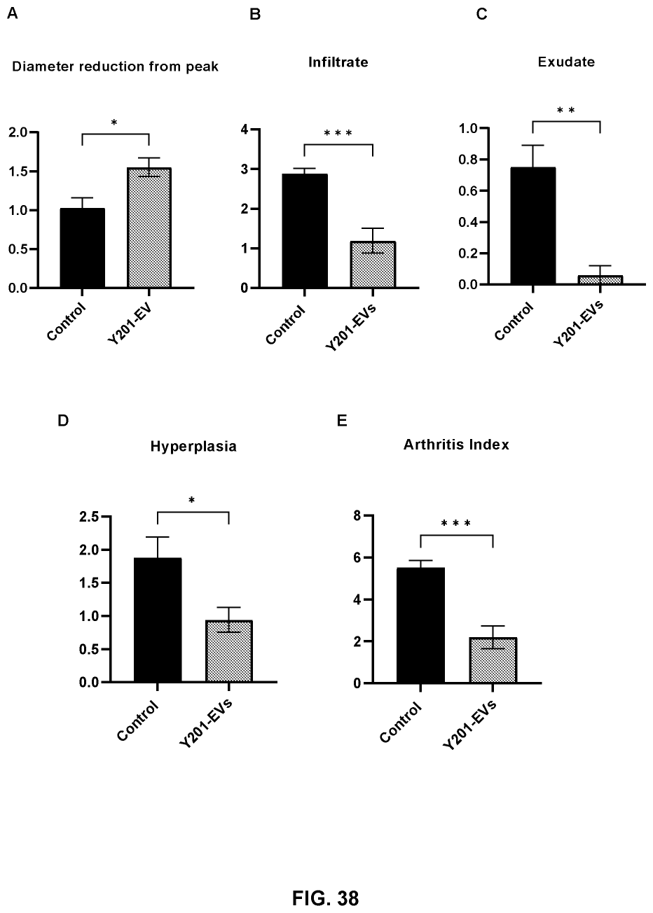

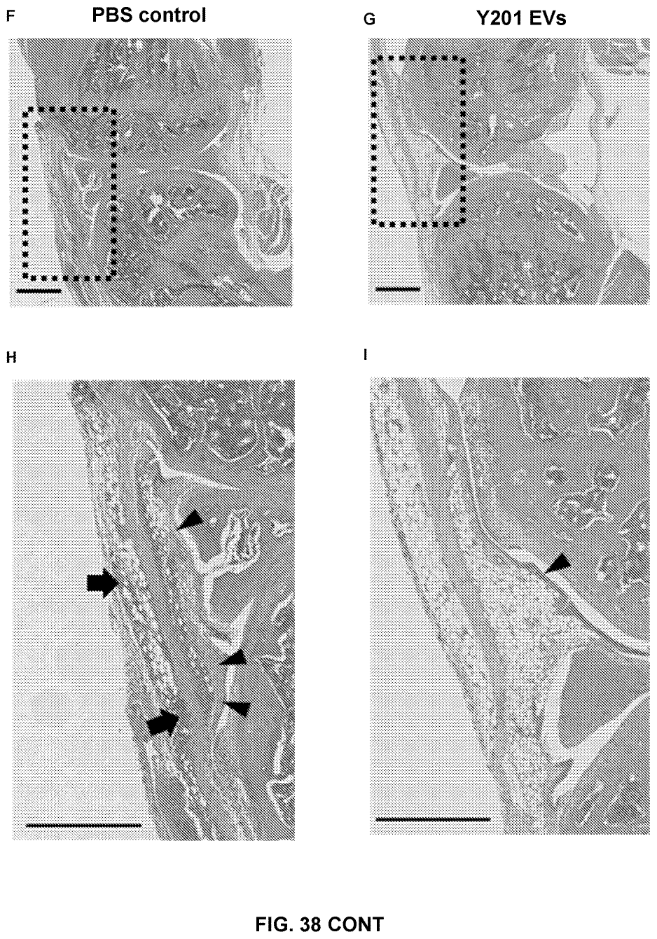

The inventors tested the bioactivity of Y201 EVs in a disease-relevant in vivo model of adjuvant-induced arthritis model following intra-articular EV injection. Following histological examination and blind scoring, the inventors demonstrated that EVs derived from Y201 MSCs induced a significant decrease in all measures of disease activity compared to vehicle controls, including joint swelling, synovial infiltrate, joint exudate, synovial hyperplasia and overall arthritis index (FIG. 38A-E). Representative haematoxylin and eosin-stained sections provide evidence of changes in synovial infiltrate and hyperplasia in control and Y201 EV-treated samples (FIG. 38F-I).

### Clonogenic and Proliferative Capacity of Wild-Type (WT) Y201 Versus FGFR3 Knockout (KO)

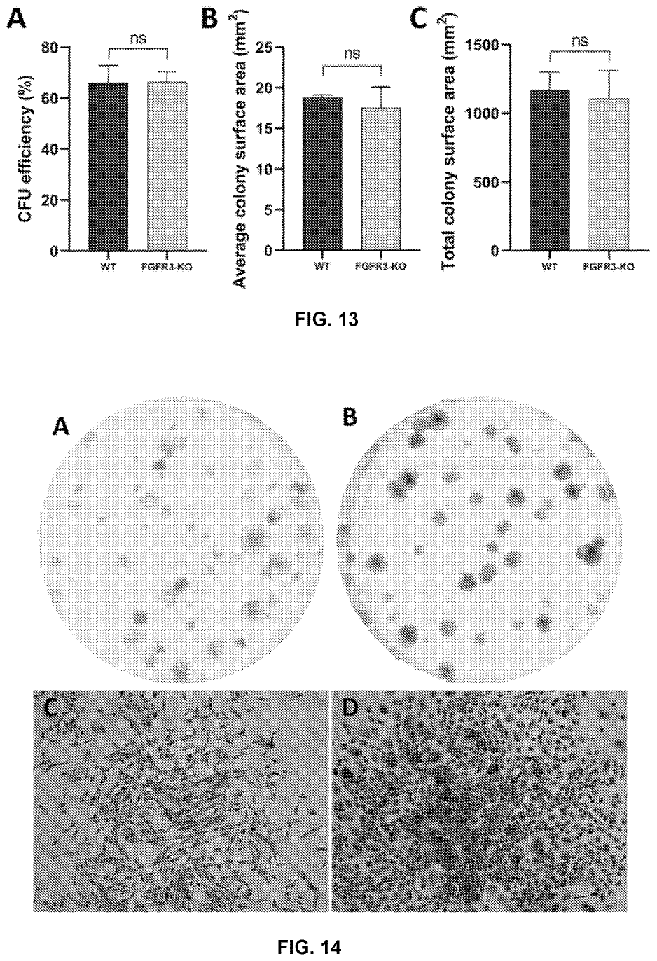

Both WT and FGFR3-KO MSCs were able to form colonies, with no significant difference in their colony forming efficiency (FIG. 13A, p=0.9665), indicating a similar adhesion and survival rate between the MSC lines. There was no significant difference in the average colony surface area (FIG. 13B, p=0.6760), indicating FGFR3-KOs have equivalent clonogenic capacity to WT MSCs. Thus, the overall surface area covered by colonies was similar for both lines (FIG. 13C, p=0.5940). However, within individual colonies there was a clear difference in the density of cells. FGFR3-KO colonies were close to, or at, confluency, whereas WT colonies were composed of sparsely distributed cells. This was evidenced by the deeper, more pronounced colour of the crystal violet stain in FGFR3-KO colonies, but was especially obvious when imaging at a single-colony level (FIG. 14).

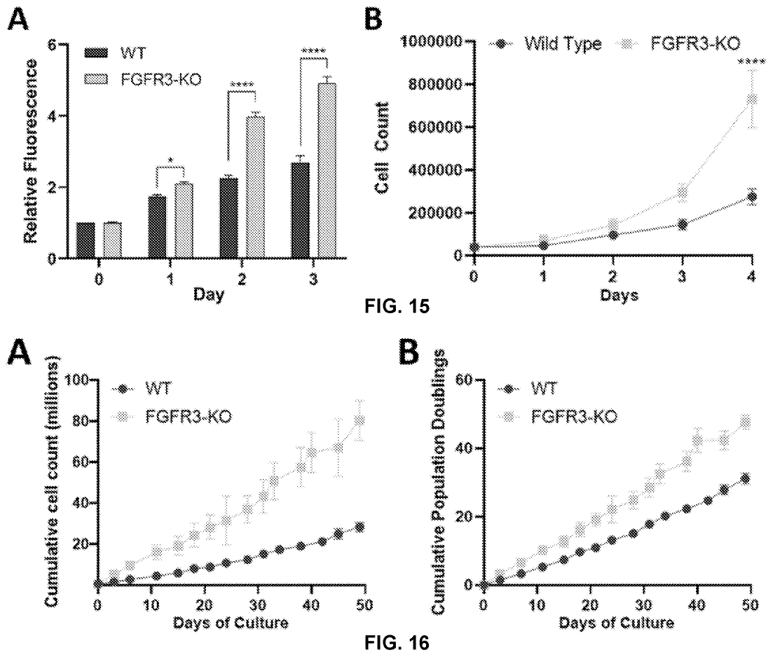

The proliferation rates of the cell lines were compared. Equal numbers of WT and FGFR3-KO cells were seeded, and Alamar Blue Cell Viability reagent utilised to give an indication of cell number over 3 days. Fluorescence readings were taken each day, with increased fluorescence being indicative of increased cell number, and thus proliferation. FGFR3-KOs show an increased fluorescence from day 1, though this was not significant. At days 2 and 3, FGFR3-KOs show a significantly increased fluorescence in comparison to WT cells (FIG. 15)

Cell counts were also used as a measure of population growth. This allowed the determination of population doubling times of each cell line. In the 24 hours after seeding, FGFR3-KOs show a 75% increase in cell number compared to 18% for WT cells, indicating FGFR3-KOs begin to proliferate sooner after seeding. At each timepoint, FGFR3-KO MSCs showed a greater increase in cell number than WT MSCs. After four days, FGFR3-KOs had a significantly higher cell number than WT cells (FIG. 15). During the most rapid stage of growth, FGFR3-KOs have an average population doubling time of 18.6 hours, compared to 25.0 hours for WT cells. Cumulative population doublings and cell number were also counted throughout long-term culture to show FGFR3-KO MSCs sustained this increased proliferation rate over time (FIG. 16). At every timepoint, FGFR3-KOs had undergone more population doublings than WT cells, further supporting an increased proliferation rate of FGFR3-KOs.

### Morphological Changes in FGFR3-KO Versus WT MSCs

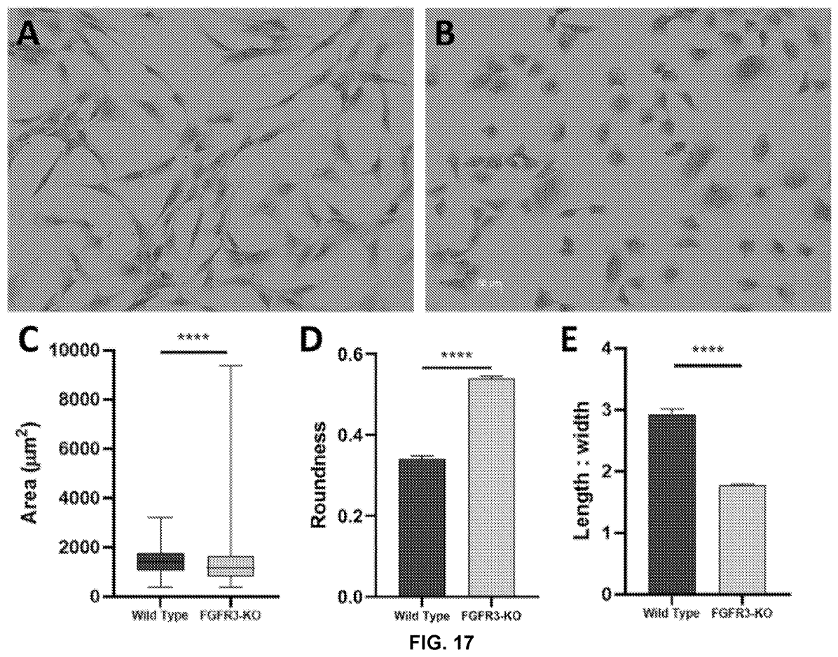

As noted in the CFU-F assay, FGFR3-KOs appeared to have an altered morphology to WT cells. However, these cells were at different density levels, with FGFR3-KOs near-confluent and WT cells more sparsely distributed. To allow comparable analysis of morphology, cells were plated at equal densities of 4000 cells per cm², stained with crystal violet, and imaged (FIG. 17A, B). CellProfiler was then used to calculate cell shape metrics. Despite no alterations to cell volume between the lines, FGFR3-KO MSCs show a significantly reduced cell area (FIG. 17C). This likely indicates that WT cells have a lower sphericity index, whereby they cover a larger area by flattening more against the growth surface. However, there is a much wider range of cell areas seen in FGFR3-KOs, with a number of large, spread cells which appear to have broad lamellipodia. FGFR3-KOs show a decreased length to width ratio and an increased roundness index (FIG. 17D, E). A high length to width ratio is seen in the WT cells which show a typical elongated, fibroblastic morphology.

### RNA-Sequencing of WT Versus FGFR3-KO MSCs

RNA-seq analysis was subjected to bioinformatic interrogation. A total of 641 transcripts had significantly differential expression between WT and FGFR3-KO MSCs, with 274 transcripts upregulated in FGFR3-KOs and 367 downregulated (Table 5 and 6).

TABLE 5
Top 200 RNA transcripts upregulated in FGFR3 KO MSCs.
RNA-seq data yielded a list of differentially expressed
transcripts between WT and FGFR3-KO MSCs, defined as those
with a log2 fold change of >1 and an adjusted p value of <0.05.
Transcripts were then divided into two gene lists corresponding
to: upregulation or downregulation in FGFR3-KOs versus WT, and
those which had <5FPKM reads in both cell lines were excluded.
Top 200 Upregulated in FGFR3 KO MSCs

| | | | |
|---|---|---|---|
| CFH | NET1 | EHD3 | GLT8D2 |
| CLCA2 | MME | LRRN4CL | GMPR |
| CD24 | CLU | TGFBR2 | GPNMB |
| KYNU | SNED1 | ANKRD29 | ABI3BP |
| IGF2BP3 | TNFRSF19 | RAI2 | CAPS |
| TNFRSF21 | AKR1C1 | FOLR3 | PROS1 |
| BACE2 | CD163L1 | EPSTI1 | NEAT1 |
| NTNG1 | SNED1 | PKIA | ADGRL2 |
| PDE4B | TMEM158 | CMTM8 | GPNMB |
| PAMR1 | WNT5A | SSX2IP | MOXD1 |
| RP11-756G12.1 | COL14A1 | GNG11 | NEAT1 |
| ENPP2 | GPR39 | ALDH3A2 | FAM133A |
| PAMR1 | DKK1 | HGF | NTM |
| TMEM35 | BDKRB1 | CFB | FAM20C |
| GAPLINC | FJX1 | IGFBP1 | ANGPTL2 |
| MMP1 | GS1-600G8.5 | LY6E | KIAA1644 |
| COL14A1 | IFI44 | RBPJ | E2F7 |
| MAGEB1 | SVIL | SERTAD4 | ARHGAP22 |
| RP13-463N16.6 | AKR1C1 | RIPK3 | GCNT1 |
| SPTLC3 | MME | ADGRL2 | MAVS |
| MAGEA12 | NOV | KCNMA1 | IF16 |
| ETV1 | KRT14 | EHD3 | GDF6 |
| KRT16 | RGL1 | CYB5R2 | FKBP1A |
| GDF15 | GS1-600G8.5 | EHD3 | PIR |
| ENPP2 | PHLDA1 | HMGA1 | NAP1L3 |
| ETV1 | HPCAL1 | SMIM3 | PYGB |
| PAMR1 | CAMK2N1 | TTC32 | MMP11 |
| TFAP2D | ANKRD29 | ESM1 | HERC6 |
| VWA5A | SOX9 | MAPKAPK3 | HS3ST3A1 |
| ITGA2 | BDKRB1 | MIR221 | PLA2G4A |
| WNT5A | FJX1 | KCNK2 | G6PD |
| SLC6A15 | SERPINE2 | SSX2IP | NQO1 |
| ENPP2 | ADGRL4 | WNT5A | AKR7A2 |
| ETV1 | LY6E | HMGA1 | PAPPA |
| STC1 | CLU | GCNT1 | SNX30 |
| DPP4 | PTGFR | RBPJ | ARHGAP12 |
| DPP4 | CITED4 | FAM20C | NQO1 |
| FMN1 | RAI2 | FMNL2 | SH2B3 |
| TNNT1 | SECTM1 | TGFBR3 | AOX1 |
| SECTM1 | GDA | ADCY4 | C1GALT1C1 |
| DOCK4 | LRRN4CL | FZD8 | TMEM171 |
| STEAP3 | PITPNC1 | KIAA1644 | HTATSF1P2 |
| STEAP3 | SPATA18 | SRPX2 | FAM3C2 |
| CLGN | HMGA1 | TSPAN5 | FAM102A |
| DNER | AIM1 | TNFRSF1B | FAM3C2 |
| SEMA3A | VIT | PKIA | KCNN4 |
| GATA2 | CXCL8 | AKR1C3 | SEMA3F |
| MME | ACTG2 | HMGA2 | PTGER4 |
| CLIC2 | TGFBR2 | SMIM3 | DCBLD2 |
| ABI3BP | TGFBR2 | DLX2 | SVIL |

TABLE 6
Top 200 RNA transcripts downregulated in FGFR3 KO MSCs.
RNA-seq data yielded a list of differentially expressed
transcripts between WT and FGFR3-KO MSCs, defined as
those with a log2 fold change of <−1 and an adjusted
p value of <0.05. Transcripts were then divided into two
gene lists corresponding to: upregulation or downregulation
in FGFR3-KOs versus WT, and those which had <5FPKM reads
in both cell lines were excluded.
Top 200 Downregulated in FGFR3 KO MSCs

| | | | |
|---|---|---|---|
| SORBS2 | NEDD9 | DSP | GPX7 |
| ACAN | LUM | HAPLN3 | CYR61 |
| SNAI1 | LUM | FAM43A | 38596 |
| ACAN | NREP | CALD1 | PLK2 |
| CNN1 | C1orf233 | SNAI2 | PIM1 |
| HES1 | EPB41L1 | ID3 | SPOCD1 |
| ID4 | TGFBI | COL5A1 | GLS |
| LAPTM4B | TRPC4 | CYB561 | CCL2 |
| CTSK | CSRNP1 | MYADM | TMEM159 |
| ACTA2 | MFAP5 | SPARC | ALDH1A3 |
| CXCL2 | EGR1 | KLF10 | IGFBP5 |
| ELN | MFGE8 | FHL1 | ADAM12 |
| SULF1 | SEMA7A | CCDC80 | MYL9 |
| ALDH2 | PTX3 | HSPB7 | BGN |
| NOTCH3 | DDAH1 | TNS3 | PTGS2 |
| BCHE | KCTD20 | ALPK2 | C6orf132 |
| ELN | STARD4-AS1 | SPARC | ADAMTS7 |
| LIF | ASNS | EFEMP1 | PODXL |
| COL15A1 | NLRP1 | LBH | PDLIM1 |
| RABGAP1 | MFAP5 | FAM101B | DUSP1 |
| C11orf87 | CTGF | CLIP4 | MSC |
| PODN | ROR1 | ICAM1 | RCAN1 |
| COL4A1 | NFKBIZ | SORBS2 | ZNF469 |
| MDK | ARHGEF2 | SLC1A4 | MYL9 |
| ELN | COL4A2 | KCTD20 | LMOD1 |
| PDE3A | TERT | INHBA | ITM2C |
| FHL1 | MEIS3 | TP53111 | CCDC80 |
| RP11-109L13.1 | MFGE8 | COL3A1 | TGFBR3L |
| ELN | CKB | CBS | IGFBP5 |
| DEPTOR | DSP | HSPB7 | NTF3 |
| LMCD1 | LOX | NUDT11 | CDH13 |
| CEMIP | ENPP7P6 | RHOB | COL16A1 |
| GREM1 | ALDH1B1 | EFEMP1 | PODXL |
| RAMP1 | GATA6 | PTPRF | ALDH1L2 |
| LMCD1 | ZC3H12A | PSAT1 | CHD1L |
| TAGLN | DHRS3 | LINC01614 | ATF3 |
| GREM1 | NXPH3 | TRAF5 | PRUNE2 |
| NR4A1 | PDLIM2 | MASP1 | COL5A2 |
| ARSJ | GHDC | EFEMP1 | THBS1 |
| TAGLN | C1orf198 | JAG1 | NFIL3 |
| ELN | MRAS | BHLHE40 | JUNB |
| CELF2 | FABP5 | SERAC1 | OPLAH |
| ABC7-42391500H16.4 | SEMA7A | KLF10 | ITPRIP |
| DBNDD2 | SEMA7A | NR2F2 | PVR |
| DKK3 | LGMN | CHSY3 | AFAP1L1 |
| PCK2 | RHOJ | PTGIS | TPM2 |
| CEMIP | COL12A1 | PALLD | MYC |
| COL12A1 | TERT | FOXC1 | COBLL1 |
| LUM | LRRC32 | MEIS3 | CYR61 |
| OXTR | FAM43A | KLF2 | HAS2 |

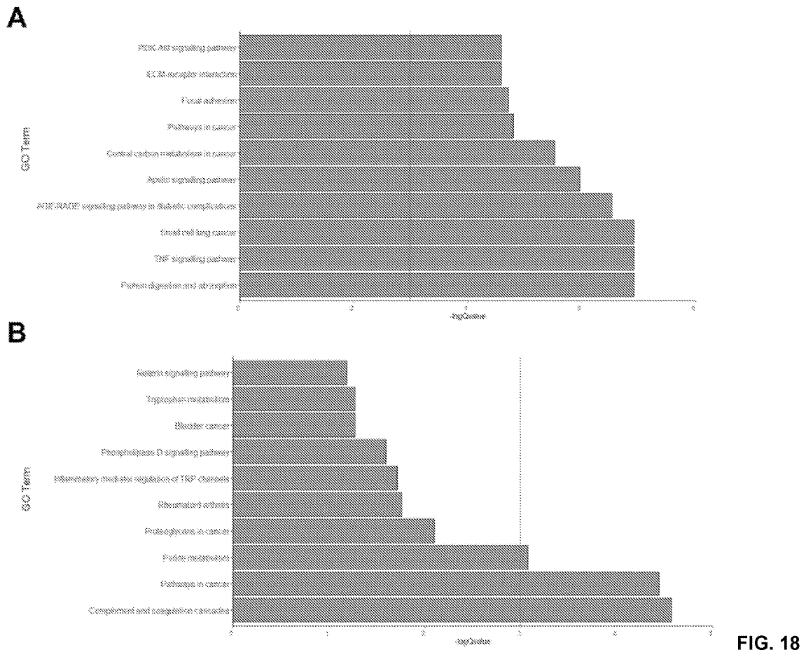

Gene ontology (GO) and KEGG pathway analysis was performed on the differentially expressed transcripts to investigate if genes that participate in particular pathways or cell behaviours are enriched. A clear result from KEGG analysis is that the gene expression changes in FGFR3-KOs overlap with genes altered in cancers, including lung and bladder (FIG. 18A). In line with this, and in agreement with the increased proliferation rate observed in FGFR3-KOs, there was a highly significant enrichment for the GO term ‘regulation of cell population proliferation’. Of the 641 genes differentially expressed in FGFR3-KOs, 103 belong to this GO term, meaning a vast number of pathways that regulate the cell cycle and proliferation have been impacted by loss of FGFR3. Additionally, transcripts encoding inflammation-related proteins were significantly upregulated in FGFR3-KOs. The most significant KEGG enrichment in the upregulated genes was ‘complement and coagulation cascades’ (FIG. 18B). As expected, there also was significant disruption to signalling pathways known to be downstream of FGFR3, such as PI3K signalling.

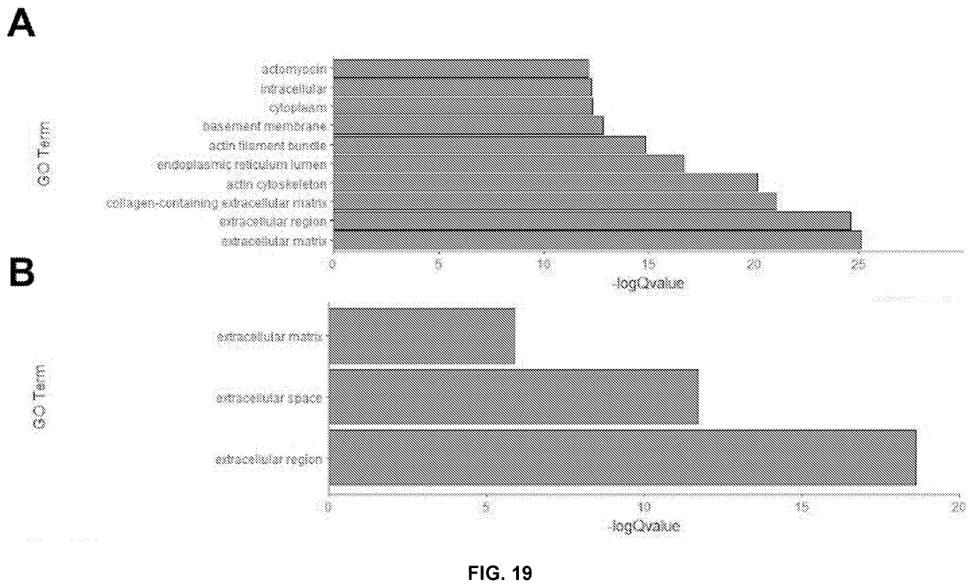

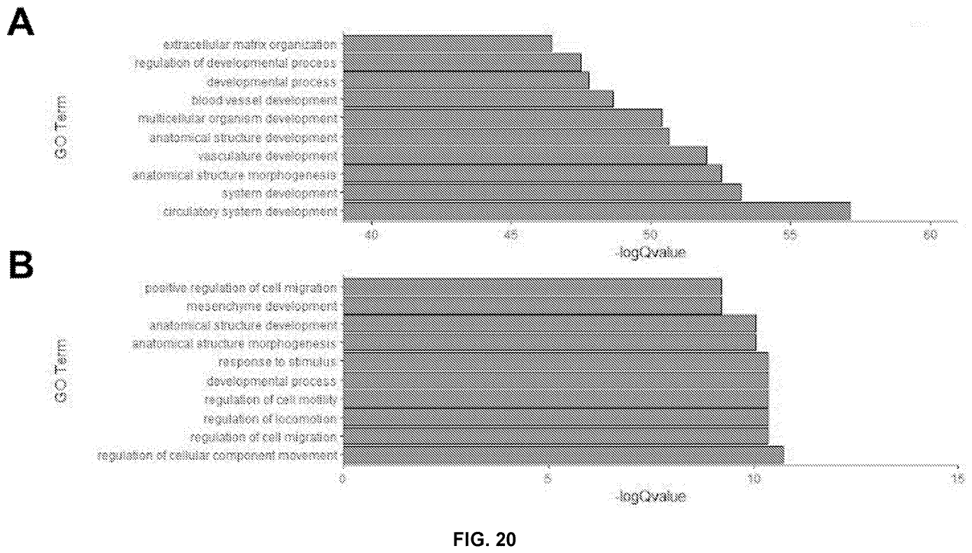

GO term Cellular Component analysis showed a significant enrichment for the actin cytoskeleton (FIG. 19A), meaning a number of transcripts encoding cytoskeletal proteins have altered expression in FGFR3-KO MSCs. This is in agreement with the altered morphology of FGFR3-KO MSCs, likely driven by changes in expression of cytoskeletal proteins. There was also significant enrichment for transcripts encoding proteins expressed extracellularly (FIG. 19B). Finally, GO Term Biological Processes analysis showed enrichment for processes predominantly relating to migration and tissue morphogenesis, and more broadly, ‘developmental process’ (FIG. 20).

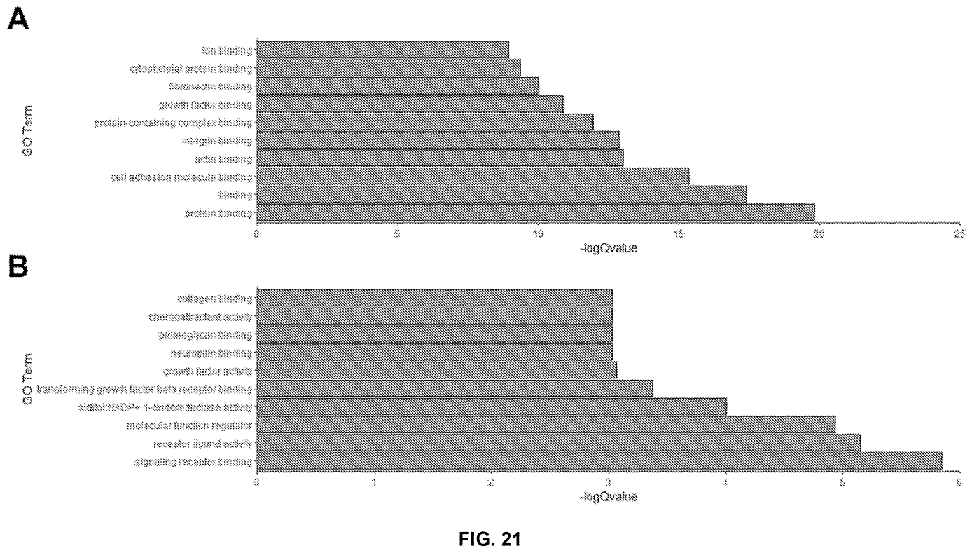

A number of proteoglycan binding genes were differentially expressed, with ‘proteoglycan binding’ being the eighth most enriched Molecular Function GO Term in the genes downregulated in FGFR3-KOs (FIG. 21).

### Phalloidin Staining of the Actin Cytoskeleton

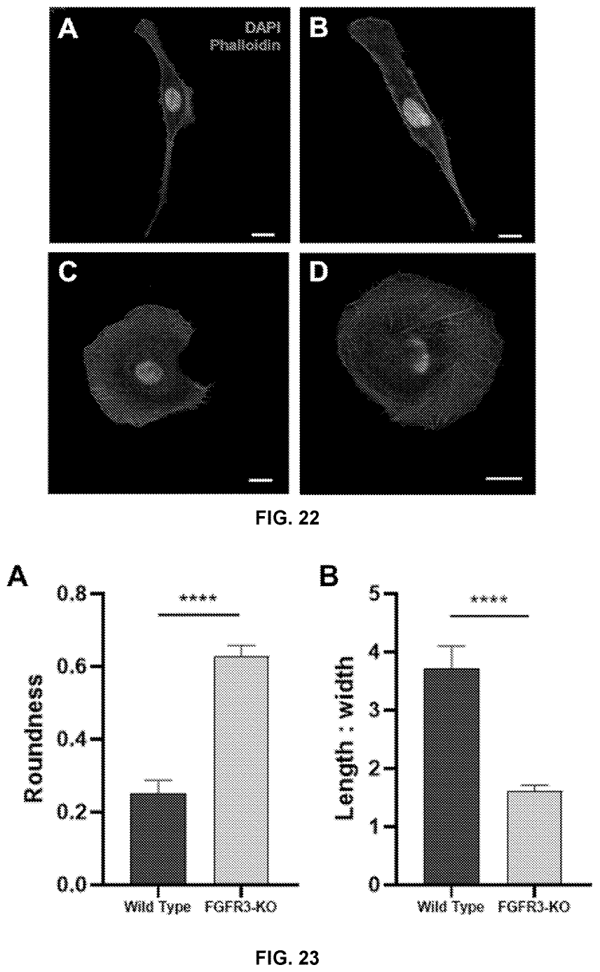

Due to the morphological changes in FGFR3-KOs, and the enrichment of actin cytoskeleton genes, fluorescence staining of the actin cytoskeleton was carried out to image the cells in greater detail. The arrangement of actin fibres in WT cells appeared striated, with stress fibres running from leading edge to tail, and some branched fibres at the leading edge (FIG. 22A, B). In FGFR3-KOs, stress fibres appeared as thicker bundles, particularly prominent on the dorsal edge behind the lamella (FIG. 22C, D). Additionally, FGFR3-KOs have a near-circular shape with prominent, broad transverse arcs and lamellipodia.

Fluorescent images were also used to quantify cell shape metrics, since the distinct contrast of the actin cytoskeleton against the background and the high magnification allowed more precise measurements of single cells. WT MSCs had a significantly increased length:width ratio, and significantly lower roundness index, comparative to FGFR3-KO MSCs (FIG. 23).

### Arp3 Immunofluorescence

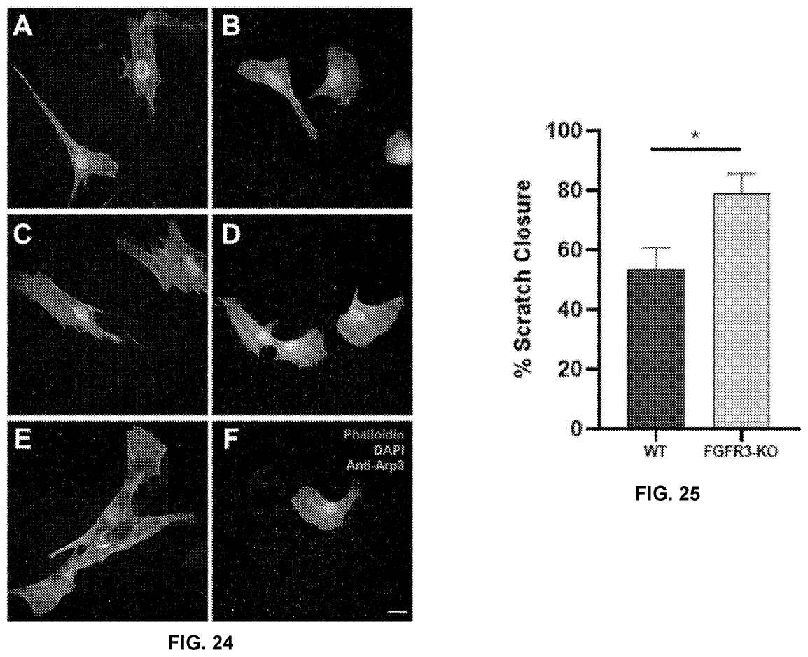

Due to its role in creating branched actin filaments, Arp3 protein is often enriched in the lamellipodia. Therefore, it was hypothesised FGFR3-KO MSCs may show a stronger localisation or increased level of Arp3 protein, allowing the formation of broad lamellipodia. However, there was widespread staining of Arp3 in FGFR3-KOs, perhaps contributing to the overall rounded shape of the cells, and there was no apparent enrichment in lamellipodia (FIG. 24). WT cells showed fewer, denser specks of Arp3 staining, many of which co-localised with actin.

### Increased Scratch Wound Healing Capacity

Due to the enrichment in the RNA-seq analysis for migratory genes being differentially expressed in FGFR3-KOs, their migratory capacity was compared to WT MSCs. WT and FGFR3-KO cells were seeded into well plates, and a scratch generated with a pipette tip. Images were taken at 0 and 24 hours, and scratch closure was calculated as a percentage of the original wound size covered by cells. FGFR3-KOs had a significantly increased closure relative to WT MSCs, indicating an increased migratory capability (FIG. 25).

### Individual Cell Migration Metrics

FGFR3-KO and WT MSCs were plated in cell culture plates with a removable insert to create a space, enabling analysis of migration without generating a wound in the cells. Cells were allowed to migrate for 24 hours, with single cell migratory metrics collected throughout the time course.

FGFR3-KOs covered a greater area at the end of the time course, despite on a single cell level having a reduced track length and velocity during migration (FIG. 26A-C). Additionally, FGFR3-KO MSCs show fewer cell divisions during the time course, suggesting increased proliferation is not the cause of the wound healing (FIG. 26D). However, despite normally having a smaller cell area than WT cells, during migration FGFR3-KOs show a significantly higher cell area, as well as a significantly decreased sphericity (FIG. 26E, F). This suggests FGFR3-KOs flatten and spread more than WT cells during migration, which aids them in covering a greater surface area. Further, FGFR3-KOs show a higher average directness, meaning they meander less than WT cells during migration, though this increase is statistically insignificant (FIG. 26G).

Image tracking of each well showed that WT MSCs undergo collective cell migration, in which a leader cell protrudes into the gap, and other cells follow. This creates a clear arc shape, maintaining cell:cell interactions (FIG. 26H). In contrast, FGFR3-KOs appear to migrate independently from each other, rather than following a leader cell, and thus swarm into the space (FIG. 261). This ability to move unhindered by, and independently from, other cells may impact the direction of migration and likely contributes to the increased gap closure.

### Characterisation and Functional Analysis of the FGFR3-KO MSC Secretome

### Characterisation of Extracellular Vesicle Size, Yield, and Morphology

### Nanoparticle Tracking Analysis

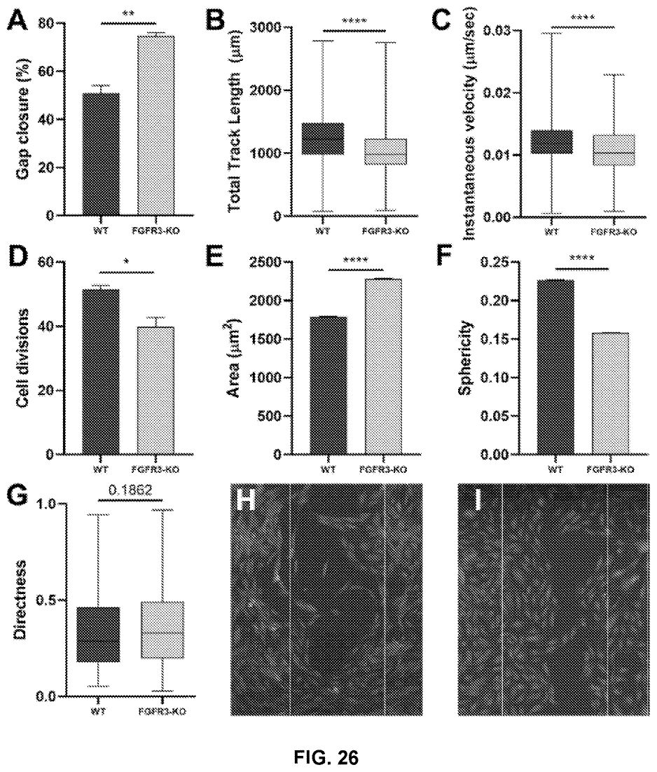

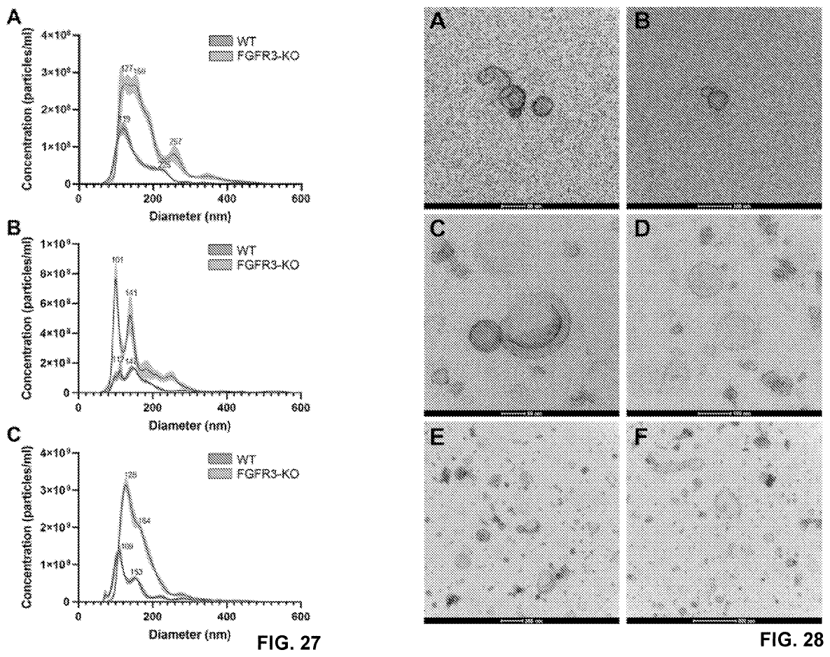

Nanoparticle tracking analysis (NTA) was performed to assess the size and number of EVs secreted by WT and FGFR3-KO MSCs. EVs were isolated by differential centrifugation yielding fractions termed 2k, 10k, and 100k, each collected at increasing centrifugation speed. In the 2k fraction, FGFR3-KO EVs had a broader size distribution than WT EVs, with a tendency towards larger diameters (FIG. 27A). The main population of WT 2k EVs had a diameter of 119 nm, whereas the FGFR3-KOs had a broad main population, ranging between 127 and 150 nm. The yield of EVs per million cells was 1.23×10⁸ and 3.10×10⁸ for WT and FGFR3-KO MSCs respectively. FGFR3-KOs therefore show an 2.52-fold increase in EV secretion in their 2k fraction. In the 10k fraction, both lines showed peaks at 101 nm, and a secondary peak at 147 nm for WT MSCs and 141 nm for FGFR3-KOs (FIG. 27B). Again, the yield in FGFR3-KOs was markedly increased. 2.98×10⁸ EVs per million cells were secreted by WT MSCs, and 9.29×10⁸ by FGFR3-KOs, meaning a 3.12-fold increase in secretion by FGFR3-KOs. For the 100k fraction, again a considerable increase in EV size was observed. WT EVs had a modal population at 105.7 nm, compared to 128 nm for FGFR3-KO EVs (FIG. 27C). Interestingly, a small peak was observed at 72 nm in the WT 100k fraction. EVs <100 nm in diameter are typically classed as exosomes, though much controversy exists in the literature. By this definition, 14% of the WT 100k fraction could be classed as exosomes, compared to less than 1% in the FGFR3-KO fraction. Again, this supports a shift towards the secretion of larger EVs as a result of the removal of FGFR3. Per million cells, the 100k fraction contained 1.70×10⁹ and 4.85×10⁹ EVs for WT and FGFR3-KO MSCs respectively. Therefore, a 2.85-fold higher yield in FGFR3-KOs.

EV size was further investigated by TEM, which demonstrated typical EV morphologies. (FIG. 28). Some EVs, however, remained intact, allowing their true spherical shape to be captured. FGFR3-KO EVs were much more abundant than WT ones, which was expected given the higher yields obtained in NTA calculations. That said, the number of WT EVs visualised by TEM was especially low compared to FGFR3-KO EVs. The high number of FGFR3-KO EVs per field of view allowed the heterogeneity of EV size and shape to be clearly observed. Vesicles <50 nm, as well as >500 nm, were observed in the FGFR3-KO 100k fraction. In the 10k and 2k fractions, for both WT and FGFR3-KO MSCs, fewer EVs were observed. However, once again, WT EVs were consistently smaller than FGFR3-KO EVs. From this TEM and NTA data, it is evident that FGFR3-KO EVs are larger than WT EVs, implying a novel role for FGFR3 in EV biogenesis. This may be indirect, as a result of the altered morphology and cytoskeleton, or through direct alteration to the secretory pathway. Next, the functional effects of EVs and the wider secretome were studied.

### Functional Effects of Extracellular Vesicles/Conditioned Medium

### FGFR3-KO Conditioned Medium Increases Migration of WT Cells

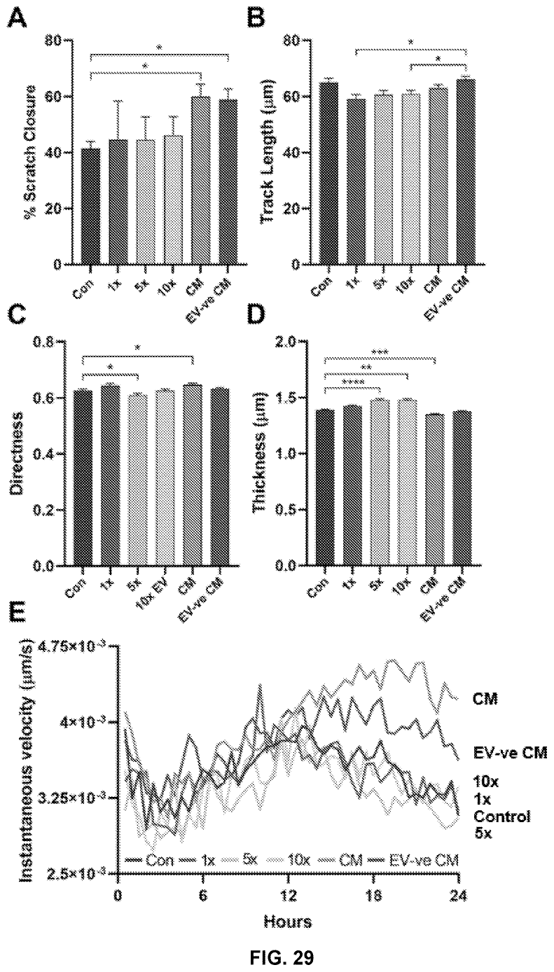

WT MSCs were used for a scratch wound assay, and either FGFR3-KO-derived EVs, conditioned media (CM), or EV-negative conditioned media (EV-ve CM), were applied. Individual cell metrics were tracked for 24 hours throughout wound healing. CM and EV-ve CM significantly increased scratch closure, relative to an untreated control (FIG. 29A). EVs at a 1×, 5×, or 10× dose had no effect on closure. No treatment had a significant effect on track length relative to the control, however the 1× and 10× EV treatments had a significantly reduced track length compared to EV-ve CM (FIG. 29B). There were minor changes to the directness index across treatments (FIG. 29C). CM significantly increased the directness, whereas 5×EVs significantly decreased directness, relative to the control. This is one parameter which likely contributed to the increased wound healing percentage. Additionally, CM treated cells showed a significantly decreased thickness (FIG. 29D), and sphericity index, meaning they flatten more against the surface to which they are adhered. In contrast, 5× and 10× EV-treated cells show a significantly increased thickness and sphericity. This may indicate uptake of EVs is associated with cells pulling away from the growth surface and becoming more spherical.

Increased velocity is likely the main factor contributing to the increased migration of CM and EV-ve CM treated MSCs. Both these treatments show significantly increased instantaneous velocity over the time course, particularly after 12 hours (FIG. 29E). The 5×EV dose shows a significantly decreased velocity, whereas 1× and 10× show a non-significant decrease, relative to the control.

These data suggest that the FGFR3-KO secretome, but not EVs, can promote MSC migration. This results from changes to MSC morphology, speed, and directness. Given its known regenerative properties, it was possible that improving scratch wound healing is a feature of MSC-CM in general. Thus, it was necessary to confirm that the FGFR3-KO secretome specifically is the driver of these changes.

### FGFR3-KO, but not WT CM, Improves Scratch Wound Healing of WT MSCs

To confirm that the FGFR3-KO secretome specifically is causing the increase in migration, another scratch wound assay was performed, using WT CM as a control. FGFR3-KO MSCs themselves were also used as a positive control, to understand if WT MSCs treated with FGFR3-KO CM can recapitulate the wound healing capacity of the mutant cells.

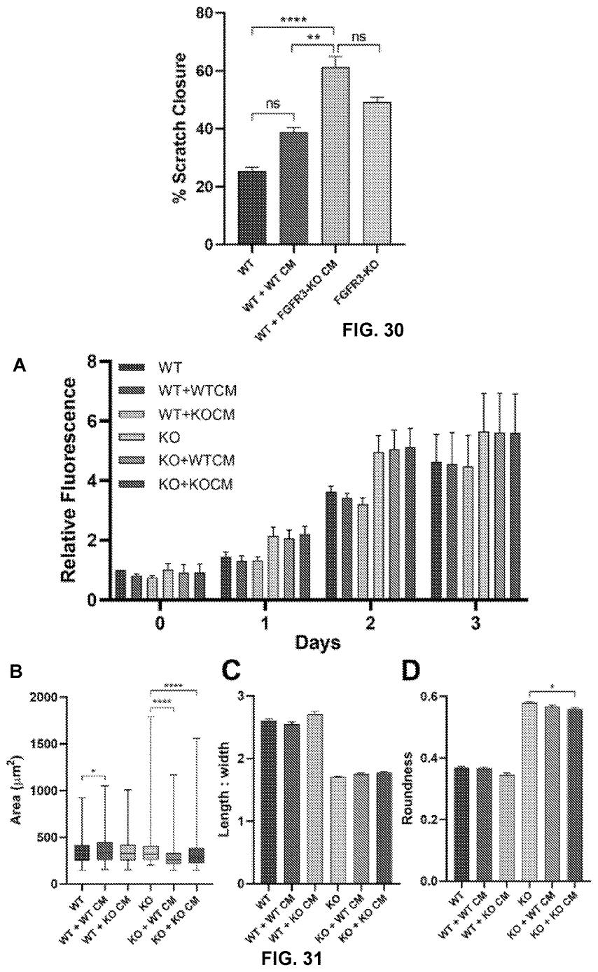

WT cells treated with WT CM showed an increased average scratch closure, but this is insignificant relative to the untreated control (p=0.0769) (FIG. 30). In contrast, WT cells treated with FGFR3-KO CM showed a 2.4-fold increase in scratch closure, significantly higher than that of both the untreated and WT-CM treated controls. In fact, treatment with FGFR3-KO CM conferred wound healing equivalent to that of FGFR3-KO cells themselves p=0.8476).

### The Effect of the MSC Secretome on MSC Proliferation and Morphology

Next, WT and FGFR3-KO MSCs were treated with either CM derived from WT or from FGFR3-KO MSCs. Proliferation was assessed using alamar blue cell viability assay, but at no time point did any CM treatment have a significant effect on viable cell numbers (FIG. 31). This indicates that the FGFR3-KO secretome can increase migration of WT MSCs, but that it is not sufficient to drive the increased proliferation seen in FGFR3-KO MSCs.

Cell morphology was also analysed following CM treatment to elucidate if the secretome contributes to the differential cell shape between the lines. WT cells showed a significant increase in cell area when treated with their own CM (FIG. 31B). This increase in area was not accompanied by any significant change to length:width ratio or roundness, indicating cells get larger without any change in shape (FIG. 31C, D). FGFR3-KO CM had no impact on WT MSC area or shape. However, treating FGFR3-KO MSCs with either WT or KO CM caused a significant decrease in cell area, especially when treated with WT CM.

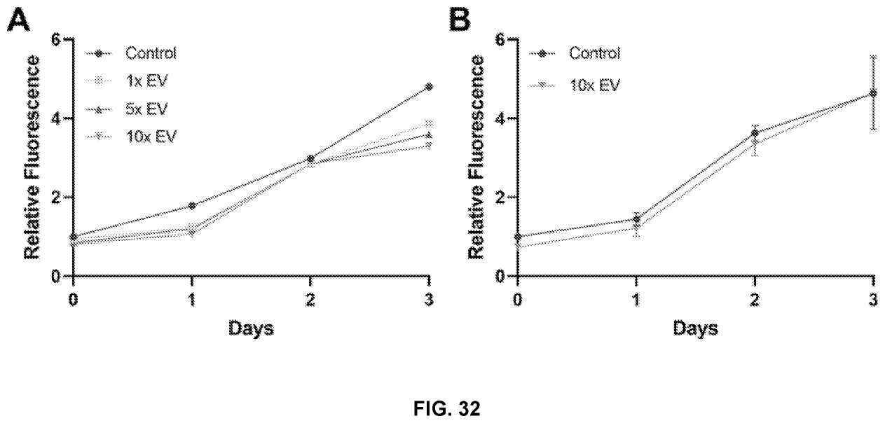

The effect of FGFR3-KO EVs on WT MSC proliferation was also assessed. Varying EV doses showed little effect of any dosage at day 2. At day 3, there was a dose-dependent decrease in viable cell numbers. Cells treated with 10× EVs had a 31% lower fluorescence than the control. This experiment was repeated with EV-depleted FBS and the 10× EV treatment applied. There was no significant effect of this treatment, indicating that neither FGFR3-KO CM or EVs regulate proliferation in MSCs (FIG. 32).

Survival and Proliferation of MSCs without Serum Supplementation

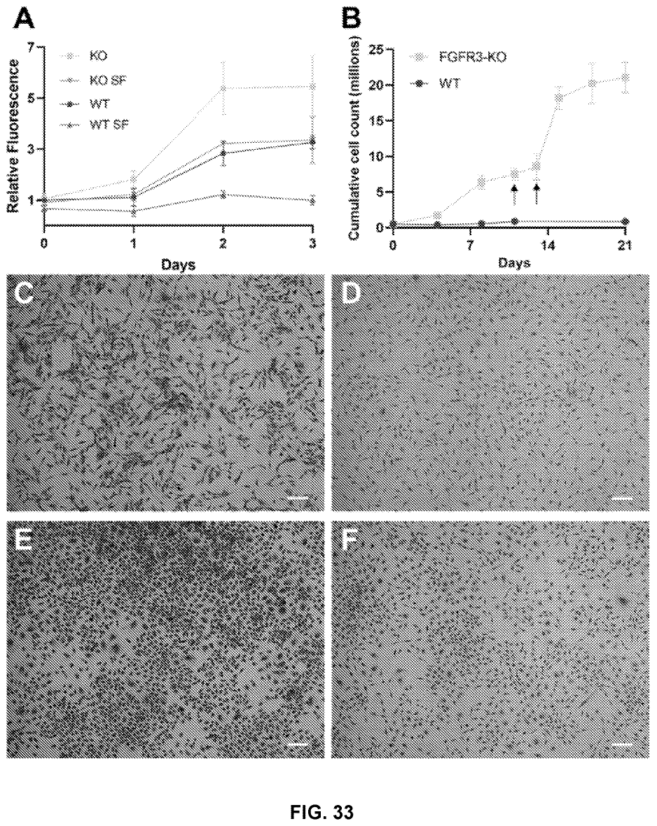

During CM collections, MSCs are cultured in serum free media to avoid contamination with FBS EVs. Culture in serum free media typically causes growth arrest, however it was noted that FGFR3-KO MSCs appeared to continue proliferating. As such, alamar blue viability assay was used to compare the growth of WT and FGFR3-KO MSCs in complete (10% FBS) and serum free (0% FBS) media. In complete media, FGFR3-KOs show a significantly higher growth than WT MSCs by day 2. By day 3, both WT and FGFR3-KO MSCs cultured in serum free media had a significantly lower growth than their complete media counterparts (FIG. 33A). Interestingly, there was no significant difference at any time point between WT MSCs in complete media and FGFR3-KO MSCs in serum free. This indicates even in the absence of serum, FGFR3-KO MSCs proliferate at a similar rate to WT cells.

To understand if FGFR3-KO MSCs could maintain growth without serum, cells were cultured continuously in serum free media over 21 days, and cell counts taken at each passage. WT MSCs showed no significant change in cell number over time, indicating a plateau of growth in the absence of serum (FIG. 33B). In contrast, FGFR3-KO MSCs showed a significant increase in cell number with time. When cells reached confluency, they were passaged equally into an additional culture flask, as indicated by arrows (FIG. 33B). This prompted a rapid increase in growth, which then slowed off as cells reached near-confluency once more. This increased survival and proliferation in the absence of serum may indicate an enriched secretome, containing anti-apoptotic factors that enable FGFR3-KO MSCs to survive.

Both WT and FGFR3-KO MSCs undergo dramatic morphological changes in the absence of serum (FIG. 33C-F). After 48 hours of culture on serum free media, WT MSCs became narrow, and spindle-like in shape, and appeared smaller in size. FGFR3-KO MSCs show a shift towards this narrow morphology, but there are still a number of rounded cells, and several with large lamellipodia.

Overall, FGFR3 KO cells give rise to good quality EVs with very low serum requirements, making said cells and EVs amenable to engineering applications. This is supported by the fact that the EVs do not have adverse effects on cells treated with them. FGFR3-KO secretome may be useful for wound healing applications, where stimulation of migration may prompt improved healing due to enhanced migration into the wound site.

Table 7 below provides non-limiting examples of characteristics of different MSC clonal lines.

TABLE 7
non-limiting examples of characteristics of different MSC clonal lines.

Cell Line

| Phenotype | FT-01 (Y201) | FT-03 (FGFR3-KO) |
|---|---|---|
| Description | ‘Stem’-like phenotype, | FGFR3 KO - Y201 |
| | exhibiting differentiation | derived; KO by CRISPR |
| | potential and | |
| | immunosuppressive activity | |
| | of what is typically referred | |
| | to as ‘MSC’ | |
| Osteogenic | +++ | + |
| Adipogenic | +++ | + |
| Chondrogenic | +++ | + |
| Immunomodulatory | +++ | |
| Migratory | +++ | ++++ |
| Growth | +++ | ++++ |
| Extracellular vesicles | Anti- | Size (NTA) |
| | inflammatory | Morphology |
| | (peritonitis in | (TEM) |
| | vivo) | |
| | Pro-migratory | |
| | (in vitro scratch | |
| | assay) | |
| | Proteomics | |
| | (MS) | |
| | miRNA | |
| | (NanoString) | |
| | Size (NTA) | |
| | Morphology | |
| | (TEM) | |
| | Marker profiles | |
| | (WB) | |
| Potential Therapeutic | Juvenile | siRNA |
| application | Idiopathic | delivery |
| | Arthritis | Small |
| | Rheumatoid | molecule |
| | arthritis | delivery |
| | Osteoarthritis | Peptide |
| | | delivery |

### Example 2

EVs may be isolated in accordance with the following protocol which was used to generate the 100K fraction described in Example 1 above.

The evening before EV isolation put a Ty45i rotor in a cold room (4 °C) to chill and the media pots containing conditioned medium (e.g. which may be prepared as described elsewhere herein e.g. using Y201 cells or FGFR3 KO cells) at 4 °C to thaw slowly overnight. The TLA100.3 rotor can be placed at 4 °C the morning of the isolation.
1. All steps should be performed quickly with efforts made to keep media cool at all times, transport tubes on ice at all times and set up all centrifuges at 4 °C before starting the isolation. Some of these steps can be ignored if one is not collecting all of the fractions or media with or without EVs that act as controls in functional assays.
2. Media is transferred from the media pots in 50 ml falcon tubes and centrifuged at 300 g for 5 minutes to remove any large cells or debris using a centrifuge (e.g. a standard bench top centrifuge).
3. At this stage, the appropriate volume of CM that will be used as a control for functional assays can be collected.
4. The rest of the supernatant is transferred into 6 new falcon tubes and centrifuged again for 20 minutes at 2000 g and 4 °C using a centrifuge (e.g. a standard bench top centrifuge).
5. The supernatant is transferred to Ty45i rotor compatible ultracentrifuge tubes and the pellet is kept. Re-suspend pellets using 600 μl cold filtered HQ PBS per tube. At the end, pool together the re-suspended pellets of the six tubes in pairs (total volume of 1200 μl per pair) in 3×1.5 ml eppendorf tube. Vigorous pipetting may be required and time taken to ensure the pellet is fully re-suspended.
6. The re-suspended pellet from the 2,000 g spin is centrifuged again at 2,000 g, 4 °C on a bench centrifugation and supernatant is aspirated/poured off, being careful not to disturb the pellet. Any bench centrifuge that has temperature control system may be used.
7. Re-suspend pellets in 50-100 μl of cold HQ-PBS (e.g. GIBCO branded PBS bought in as 1× bottles as this is ‘particle free’) and transfer the suspension in a protein Low-binding tube. Freeze re-suspended pellets at −70 °C (2K fraction).
8. The supernatant that was previously transferred to the 6×Ty45i ultracentrifuge tubes must be paired and tubes must be balanced to within 0.01 g of each other (small pieces of autoclave tape can be used for balancing the tubes if the difference is below 0.009). Tubes should be inspected for signs of excessive wear and damage before use, all O-rings must be in place. Tubes should be weighed with all caps, O-rings and metal lids on as these all have minor differences in weight, this can be done on a AND FX300i Balance for example. Tubes must be filled to at least ⅔ of the maximum volume. Media is centrifuged in a floor standing ultracentrifuge (e.g. with the text on each tube facing outwards in order to make identifying the pellet easier) (Machine 100XP, Beckman) at 10,000 g (9,000 rpm in Ty45i rotor) at 4 °C for 45 minutes.
9. Supernatant is transferred to fresh Ty45i ultracentrifuge tubes and the pellet is collected. Re-suspend pellets using 600 μl cold filtered HQ-PBS per tube. At the end pool together the re-suspended pellets of the six Ty45i tubes in pairs (total volume of 1200 μl per pair) in 3×1.5 ml micro ultra-centrifuge tubes (Beckman Microfuge Tube Polyallomer cat no. 357448). Vigorous pipetting may be required and time taken to ensure the pellet is fully re-suspended.
10. The re-suspended pellet from the 10,000 g spin is centrifuged again at 10,000 g (16,000 rpm for the TLA100.3 rotor) for 45 mins at 4 °C using a TLA100.3 rotor in TL100 μlracentrifugation and supernatant is aspirated/poured off being careful not to disturb the pellet.
11. Re-suspend pellets in 50-100 μL of HQ-PBS and transfer the suspension in a protein Low-binding tube. Store at −70 °C (10K fraction).
12. The supernatant that was transferred to 6× fresh Ty45i ultracentrifuge tubes must be balanced to within 0.01 g of each other and filled to at least ⅔ of the maximum volume. Media is centrifuged in a floor standing ultracentrifuge at 100,000 g (30,000 rpm in Ty45i rotor, Machine 100XP, Beckman) at 4 °C for 90 minutes (e.g. with the text on each tube facing outwards in order to make identifying the pellet easier). A small pellet may be visible, try to move tubes gently so as not to disturb the pellet.
13. Pour off the supernatant gently so as not to disturb the pellet. If one is performing functional assays and want to use the supernatant as a negative control, collect the appropriate amount in a new 50 ml falcon tube and store it in-70 °C. Aspirate any remains left behind using a pipette.
14. Re-suspend pellets using 600 μl cold filtered PBS per tube. At the end pull together the re-suspended pellets of the six Ty45i tubes in pairs (total volume of 1200 μl per pair) in 3×1.5 ml micro ultra-centrifuge tubes. Vigorous pipetting may be required and time taken to ensure the pellet is fully re-suspended
15. Micro-ultracentrifuge tubes should have at least 1 mL in before spinning
16 Spin micro-ultracentrifuge tubes at 100,000 g (45,000 rpm for the TLA100.3 rotor) for 90 mins.
17. Aspirate/pour off supernatant being careful not to disturb pellet.
18. Re-suspend pellets in 50-100 μL of HQ-PBS and transfer the suspension in a protein Low-binding tube (100k Fraction).
19. Freeze re-suspended pellets at −70 °C.

The reader's attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings), may be replaced by alternative features serving the same, equivalent, or similar purpose, unless expressly stated otherwise. Thus, unless expressly stated otherwise, each feature disclosed is one example only of a generic series of equivalent or similar features.

The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

### REFERENCES

James S, Fox J, Afsari F, Lee J, Clough S, Knight C, Ashmore J, Ashton P, Preham O, Hoogduijn M, Ponzoni Rde A, Hancock Y, Coles M, Genever P. Multiparameter Analysis of Human Bone Marrow Stromal Cells Identifies Distinct Immunomodulatory and Differentiation-Competent Subtypes. Stem Cell Reports. 2015 Jun. 9; 4 (6): 1004-15. doi: 10.1016/j.stemcr.2015.05.005.
Wilson A J, Rand E, Webster A J, Genever P G. Characterisation of mesenchymal stromal cells in clinical trial reports: analysis of published descriptors. Stem Cell Res Ther. 2021 Jun. 22; 12 (1): 360. doi: 10.1186/s13287-021-02435-1.
Alasdair G Kay, James M Fox, James Hewitson, Andrew Stone, Sophie Robertson, Sally James, Xiao-nong Wang, Elizabeth Kapasa, Xuebin Yang, Paul G Genever Identification of CD317-Positive Pro-inflammatory Immune Stromal Cells in Human Mesenchymal Stromal Cell Preparations. bioRxiv 2022.02.10.479972; doi: https://doi.org/10.1101/2022.02.10.479972

Sequences

SEQ ID NO: 1-CACCGCATCCGGCAGACGTACACGC
SEQ ID NO: 2-AAACGCGTGTACGTCTGCCGGATGC
SEQ ID NO: 3-
CAGCACCGCCGTCTGGTTGGCCGGCAGCCCCGCCTGCAGGATGGGCCGGT
GCGGGGAGCACTCCAGCACGTCCAGCGTGTACGTCTGCCGGATGCTGCCA
AACTTGTTCTCCACGACGCA
SEQ ID NO: 4-CAC GGC CCA GCT CTG AGA AAG
SEQ ID NO: 5-ACC CAA ATC CTC ACG CAA CC
SEQ ID NO: 6-CAT CAA GCC ATC CAC TAT ACA GAGG CGT
CTG GGA GAC ATA CA
SEQ ID NO: 7-CTA CAC TGT CCA CCA TACTTA TGC TGG
ATG TAT GGG GCT
SEQ ID NO: 8-CCT GTC CTC CAT TCA CCC ACA C
SEQ ID NO: 9-GAG TTC AGT GAA GGG GAG CC
SEQ ID NO: 10-CTG AGA AAG AGG TCA GGA G
SEQ ID NO: 11-CTG CTA GAG CAG GAG TGA GG
SEQ ID NO: 12-CGG GTC CCA CTC CTA GAC AC
SEQ ID NO: 13-CGG CCC TTA CCG ATA CTT CAT
SEQ ID NO: 14-CAA TAA ACG TTG GGT GCC GC
SEQ ID NO: 15-CTC AGT AAG TGC TGG CCT CTG
SEQ ID NO: 16-GRGDSP
SEQ ID NO: 17-GRADSP

1. A mesenchymal stem cell (MSC)-like cell deposited under European Collection of Authenticated Cell Cultures accession number 22072103 or a derivative thereof.

2. The MSC-like cell of claim 1, wherein the derivative has reduced FGFR3 expression and/or activity compared to a MSC-like cell deposited under European Collection of Authenticated Cell Cultures accession number 22072103.

3. The MSC-like cell of claim 1, wherein the derivative only differs from a MSC-like cell deposited under European Collection of Authenticated Cell Cultures accession number 22072103 by having reduced FGFR3 expression and/or activity.

4. The MSC-like cell of any claim 2, wherein the derivative;
(a) has increased levels of at least 10 different RNA transcripts selected from Table 5 compared to the levels of the same RNA transcripts in a mesenchymal stem cell (MSC)-like cell deposited under European Collection of Authenticated Cell Cultures accession number 22072103; and/or
(b) has reduced levels of at least 10 different RNA transcripts selected from Table 6 compared to the levels of the same RNA transcripts in a mesenchymal stem cell (MSC)-like cell deposited under European Collection of Authenticated Cell Cultures accession number 22072103.

5. The mesenchymal stem cell (MSC)-like cell or derivative thereof of claim 1, wherein the MSC-like cell or derivative thereof is deposited under European Collection of Authenticated Cell Cultures accession number 22072101, or a derivative thereof.

6. A cell population comprising a plurality of cells of claim 1.

7. A DNA preparation comprising the genomic DNA of the cell of claim 1.

8. A cell-free conditioned medium that is obtainable by cell culture of the cell of claim 1 or a plurality of cells of claim 1 optionally, wherein the cell-free conditioned medium is depleted or substantially free of extracellular vesicles (EVs).

9. (canceled)

10. (canceled)

11. A secretome, or a portion thereof, that is obtainable by cell culture of the cell of claim 1 or a plurality of cells of claim 1 optionally, wherein the secretome, or the portion thereof, is depleted or substantially free of extracellular vesicles (EVs).

12. (canceled)

13. (canceled)

14. An extracellular vesicle (EV) population that is obtainable by cell culture of the cell of claim 1 or a plurality of cells of claim 1.

15. The EV population of claim 14, wherein the EV population is a 100K EV fraction.

16. A cell-free conditioned medium or secretome or portion thereof obtained by cell culture of the cell of claim 1 or a plurality of cells of claim 1, comprising at least 10 different proteins selected from Table 2.

17. A cell-free conditioned medium, secretome or portion thereof, or EV population obtained by cell culture of the cell of claim 1 or a plurality of cells of claim 1, comprising at least 10 different proteins selected from Table 3.

18. A cell-free conditioned medium, secretome or portion thereof, or the EV population obtained by cell culture of the cell of claim 1 or a plurality of cells of claim 1, comprising at least 10 different proteins selected from Table 3 at least 10 different miRNA selected from Table 4.

19. A composition comprising at least 10 different proteins selected from Table 2, at least 10 different proteins selected from Table 3, or at least 10 different miRNAs selected from Table 4; optionally, wherein the composition is a cell-free composition.

20. (canceled)

21. (canceled)

22. (canceled)

23. A pharmaceutical composition comprising;
(a) the MSC-like cell of claim 1; a cell population of a plurality of cells of claim 1; a DNA preparation, a cell-free conditioned medium, a secretome or a portion thereof, or an extracellular vesicle (EV) population obtained by cell culture of the cell of claim 1 or a plurality of cells of claim 1; or a composition comprising at least 10 different proteins selected from Table 2, at least 10 different proteins selected from Table 3, or at least 10 different miRNAs selected from Table 4, optionally, wherein the composition is a cell-free composition; and
(b) a pharmaceutically acceptable adjuvant, excipient, diluent, and/or carrier.

24. (canceled)

25. A method for promoting tissue repair a subject in need thereof, comprising administering to the subject the pharmaceutical composition of claim 23.

26. The method of claim 25, wherein the pharmaceutical composition is administered for treating or preventing a disease or condition in the subject associated with cartilage damage, optionally wherein the disease or condition associated with cartilage damage is arthritis, optionally wherein the arthritis is selected from the group consisting of: juvenile idiopathic arthritis, rheumatoid arthritis, osteoarthritis, spondyloarthritis and psoriatic arthritis.

27. (canceled)

28. The method of claim 25, wherein the pharmaceutical composition is administered to the subject for wound healing and/or tissue regeneration.

29. A method for treating or preventing inflammation in a subject in need thereof, comprising administering to the subject the pharmaceutical composition of claim 23.

30. The method of claim 29, wherein the pharmaceutical composition is administered to the subject for use in treating or preventing an autoimmune disease or condition.

31. A method to deliver cargo to a cell, comprising administering to the cell the Use of an extracellular vesicle (EV) population of claim 14.

32. A method of screening a test compound for its ability to induce differentiation of MSC-like cells, said method comprising: a) contacting the test compound with the MSC-like cell of claim 1 or a plurality of cells of claim 1, and b) determining the effect of the test compound on at least one marker of differentiation.

33. The method of claim 32, wherein the at least one marker of differentiation is selected from the group consisting of: an osteogenic differentiation marker and a chondrogenic differentiation marker, optionally wherein:
a) the osteogenic differentiation marker is selected from the group consisting of: Type I collagen, alkaline phosphatase, BMP2, osteopontin, osteonectin, osteocalcin, Bone Sialoprotein, and Runx2; and/or
b) the chondrogenic differentiation marker is selected from the group consisting of: Type II collagen, Type X collagen, aggrecan, lubricin, cartilage oligomeric matrix protein, and Sox9.

34. (canceled)


SEQUENCE LISTING (17 sequences)

SEQ ID NO: 1 - DNA, 25 bp, synthetic construct
  source 1..25: mol_type = other DNA
  misc_feature 1..25: note = sgRNA Fwd
caccgcatccggcagacgtacacgc

SEQ ID NO: 2 - DNA, 25 bp, synthetic construct
  source 1..25: mol_type = other DNA
  misc_feature 1..25: note = sgRNA Rev
aaacgcgtgtacgtctgccggatgc

SEQ ID NO: 3 - DNA, 120 bp, synthetic construct
  source 1..120: mol_type = other DNA
  misc_feature 1..120: note = ssODN
cagcaccgccgtctggttggccggcagccccgcctgcaggatgggccggtgcggggagcactccagcacgtccagcgtgtacgtctgccggatgctgccaaacttgttctccacgacgca

SEQ ID NO: 4 - DNA, 21 bp, synthetic construct
  source 1..21: mol_type = other DNA
  misc_feature 1..21: note = FGFR3 CRISPR/Cas9 Validation Forward Primer
cacggcccagctctgagaaag

SEQ ID NO: 5 - DNA, 20 bp, synthetic construct
  source 1..20: mol_type = other DNA
  misc_feature 1..20: note = FGFR3 CRISPR/Cas9 Validation Reverse Primer
acccaaatcctcacgcaacc

SEQ ID NO: 6 - DNA, 42 bp, synthetic construct
  source 1..42: mol_type = other DNA
  misc_feature 1..42: note = FGFR3 Offtarget 1 Forward Primer
catcaagccatccactatacagaggcgtctgggagacataca

SEQ ID NO: 7 - DNA, 39 bp, synthetic construct
  source 1..39: mol_type = other DNA
  misc_feature 1..39: note = FGFR3 Offtarget 1 Reverse Primer
ctacactgtccaccatacttatgctggatgtatggggct

SEQ ID NO: 8 - DNA, 22 bp, synthetic construct
  source 1..22: mol_type = other DNA
  misc_feature 1..22: note = FGFR3 Offtarget 2 Forward Primer
cctgtcctccattcacccacac

SEQ ID NO: 9 - DNA, 20 bp, synthetic construct
  source 1..20: mol_type = other DNA
  misc_feature 1..20: note = FGFR3 Offtarget 2 Reverse Primer
gagttcagtgaaggggagcc

SEQ ID NO: 10 - DNA, 19 bp, synthetic construct
  source 1..19: mol_type = other DNA
  misc_feature 1..19: note = FGFR3 Offtarget 3 Forward Primer
ctgagaaagaggtcaggag

SEQ ID NO: 11 - DNA, 20 bp, synthetic construct
  source 1..20: mol_type = other DNA
  misc_feature 1..20: note = FGFR3 Offtarget 3 Reverse Primer
ctgctagagcaggagtgagg

SEQ ID NO: 12 - DNA, 20 bp, synthetic construct
  source 1..20: mol_type = other DNA
  misc_feature 1..20: note = FGFR3 Offtarget 4 Forward Primer
cgggtcccactcctagacac

SEQ ID NO: 13 - DNA, 21 bp, synthetic construct
  source 1..21: mol_type = other DNA
  misc_feature 1..21: note = FGFR3 Offtarget 4 Reverse Primer
cggcccttaccgatacttcat

SEQ ID NO: 14 - DNA, 20 bp, synthetic construct
  source 1..20: mol_type = other DNA
  misc_feature 1..20: note = FGFR3 Offtarget 5 Forward Primer
caataaacgttgggtgccgc

SEQ ID NO: 15 - DNA, 21 bp, synthetic construct
  source 1..21: mol_type = other DNA
  misc_feature 1..21: note = FGFR3 Offtarget 5 Reverse Primer
ctcagtaagtgctggcctctg

SEQ ID NO: 16 - protein, 6 aa, synthetic construct
  REGION 1..6: note = RGD blocking peptide
GRGDSP

SEQ ID NO: 17 - protein, 6 aa, synthetic construct
  REGION 1..6: note = RAD control peptide
GRADSP